SEQUENCE LISTING (52 sequences)

SEQ ID NO: 1 - DNA, 20 bp, Artificial Sequence
  V-1, primer to amplify a 751 bp fragment from CVH transcript
gctcgatatgggttttggat

SEQ ID NO: 2 - DNA, 21 bp, Artificial Sequence
  v-2 , primer to amplify a 751 bp fragment from CVH transcript
ttctcttgggttccattctgc

SEQ ID NO: 3 - DNA, 20 bp, Artificial Sequence
  Dazl-1, primer to amplify a 536 bp fragment from Dazl transcript
gcttgcatgcttttcctgct

SEQ ID NO: 4 - DNA, 19 bp, Artificial Sequence
  Dazl-2, primer to amplify a 536 bp fragment from Dazl transcript
tgcgtcacaaagttaggca

SEQ ID NO: 5 - DNA, 21 bp, Artificial Sequence
  Act-RT-1, primer to amplify a 597 bp fragment from chicken actin transcript
aacaccccagccatgtatgta

SEQ ID NO: 6 - DNA, 20 bp, Artificial Sequence
  Act-RT-2, primer to amplify a 597 bp fragment from chicken actin transcript
tttcattgtgctaggtgcca

SEQ ID NO: 7 - DNA, 28 bp, Artificial Sequence
  Hs4-Bam-F, PCR primer
aggatccgaagcaggctttcctggaagg

SEQ ID NO: 8 - DNA, 30 bp, Artificial Sequence
  HS4-Bgl-R, PCR Primer
aagatcttcagcctaaagctttttccccgt

SEQ ID NO: 9 - DNA, 45 bp, Bacteriophage phi-C31
  misc_feature: attB site, junction region
tgcgggtgccagggcgtgcccttgggctccccgggcgcgtactcc

SEQ ID NO: 10 - DNA, 44 bp, Gallus
  misc_feature: cell line NLS1-41, junction region
gcgggtgccagggcgtgccccgctggggccgcactccttcatca

SEQ ID NO: 11 - DNA, 45 bp, Gallus
  misc_feature: cell line 5-7, junction region
tgcgggtgccagggcgtgccatggtcctgtaggagtatgacaggc

SEQ ID NO: 12 - DNA, 45 bp, Gallus
  misc_feature: cell line 19-1-1, junction region
tgcgggtgccagggcgtggaagccccttggtctcagcagtgacac

SEQ ID NO: 13 - DNA, 45 bp, Gallus
  misc_feature: cell line 18-4-11, junction region
tgcgggtgccagggcgtgccctaaccaagggctgaccgggacaag

SEQ ID NO: 14 - DNA, 45 bp, Gallus
  misc_feature: cell line 18-3-43, junction region
tgcgggtgccagggcgccatggagaccccaatggccccatttaac

SEQ ID NO: 15 - DNA, 45 bp, Gallus
  misc_feature: cell culture 18-5-36, junction region
tgcgggtgccagggcgtgccatcctatggcatcctatagaaccct

SEQ ID NO: 16 - DNA, 45 bp, Gallus
  misc_feature: cell culture 18-5-36-3, junction region
tgcgggtgccagggcgtgcccactatggggtcctacaaccactat

SEQ ID NO: 17 - DNA, 45 bp, Gallus
  misc_feature: cell culture NLS2-47, junction region
tgcgggtgccagggcgtgctggggtcctacaaccactatggggcc

SEQ ID NO: 18 - DNA, 45 bp, Gallus
  misc_feature: cell culture NLS1-30, junction region
tgcgggtgccagggcgtgccccctatggggccattggggtctcca

SEQ ID NO: 19 - DNA, 45 bp, Gallus
  misc_feature: cell culture 18-3-12, junction region
tgcgggtgccagggcgtgcccggggtcctataaccactatggggc

SEQ ID NO: 20 - DNA, 45 bp, Gallus
  misc_feature: cell culture 19-5-21, junction region
tgcgggtgccagcatcctatggcaccctatagaaccctatggcat

SEQ ID NO: 21 - DNA, 45 bp, Gallus
  misc_feature: cell culture NLS2-38, junction region
tgcgggtgccagggcgtgcccccgggcgcgtactccacctcacac

SEQ ID NO: 22 - DNA, 252 bp, Gallus
  misc_feature: PGC clone 18-5-36-2,  insertion site
tgcgggtgccagggcgtgcccactatggggtcctacaaccactatggggccacagaaccccctatggggccctataaccactatgggggcctataaccccccatggggtcctataaccactatggggccctataaccactatggggtcctttaaccactatggggccccagaaccccctatggggccctatagccactatggggtcctataaccccccatggggtcctgtaaccactatggggccctataac

SEQ ID NO: 23 - DNA, 246 bp, Gallus
  misc_feature: PGC clone 2-47, insertion site
tgcgggtgccagggcgtgctggggtggtacaaccactatggggccacagaaccccctatggggccctataaccactatggggccctataaccccccatggggtcctataaccactatggggccctataaccactatggggtcctttaaccactatggggccccagaaccccctatggggccctatatccactatggggtcctataaccccccatggggtcctgtaaccactatggggccctataac

SEQ ID NO: 24 - DNA, 268 bp, Galllus
  misc_feature: PGC clone 18-3-12, insertion site
tgcgggtgccagggcgtgcccggggtcctataaccactatggggccacagaaccccctatggggccctataaccactatggggccctataaccccccatggggtcctgtaaccactatggggccccagaaccccctatggggccctataaccccccatggggtcctgtaaccactatggggccccagaatcccctatggggccctatagcaactatggggtcctataaccccccatggggtcctgtaaccactatggggccctataac

SEQ ID NO: 25 - DNA, 203 bp, Gallus
  misc_feature: PGC clone 18-3-43
tgcgggtgccagggcgccatggagaccccaatggccccatttaaccccactgaccccaatgtccccaacatcccctgatgtccccaatgtggccccgatgaccccatgatgtccccaatacccccaatgaccacaacgaccccatacccccctgtgaccccataccccccaatgaccccatatccccgatgccccccaacgcc

SEQ ID NO: 26 - DNA, 260 bp, Gallus
  misc_feature: PGC clone 18-5-36-1
tgcgggtgccagggcgtgccatcctatggcatcctatagaaccctatggcaccaaatggcatcctatagcacccaatggcagccaatggcaccctatggcatcctatagcatcccatggcatcctatagcaccccatggcatcctagagcaccctatggcaccctatggcatcctatggcaccctatagaacccaatggcatcctatggcatcctatagcagcctatggcaccctatagcaccctatggcaccctatggc

SEQ ID NO: 27 - DNA, 263 bp, Gallus
  misc_feature: PGC clone 19-5-21 insertion site
tgcgggtgccagcatcctatggcaccctatagaaccctatggcaccaaatggcatcctatagcacccaatggcagccaatggtatcctatggcatcctatggcacccaatggcaccctatggcatcctatggcacccaatggcatcctatgccatcatatggcatcctatggcacccaatggcatcctatggcatcctatggcatcctatggcaccctatggcatcctatggcaccctatagaaccctatgacatcctatggc

SEQ ID NO: 28 - DNA, 257 bp, Gallus
  misc_feature: PGC clone 1-30 insertion site
tgcgggtgccagcatcctatggcaccctatagaaccctatggggtctctgtggggccattgggccctatggggccgtcggggctctatggggccataggggtctccatggggtctctctggggccattgggccctatggggccgttggggccctatgggcccattagggctctgtggggccattgggcactatttttcctttgggggccctatggggccattggggtctccacggggtctctctgggtccattgggc

SEQ ID NO: 29 - DNA, 281 bp, Gallus
  misc_feature: PO41 consensus sequence
tatggggctctatggggctctatggggctctatggggcggctatggggctctatggggctctatggggctctatggggcggctatggggctctatggggctctatggggctctatggggcggctatggggctcatggggctctatggggctctatggggcggctatggggctctatggggctctatggggctctatggggcggctatggggctctatggggctctatggggctctatggggcggcgatggggctctatggggctctatggggctctatggg

SEQ ID NO: 30 - DNA, 100 bp, Gallus
  misc_feature: attP sequence, 100 bp region of consensus repeat
cccaggtcagaagcggttttcgggagtagtgccccaactggggtaacctttgagttctctaagttgggggcgtagggtcgccgacatgacacaaggggtt

SEQ ID NO: 31 - DNA, 100 bp, Gallus
  misc_feature: PO41 sequence, 100 bp of consensus repeat
ccgtatggggctctatggggctctatggggctctatggggcggctatggggctctatggggctctatggggctctatggggcgcctatggggctctatgg

SEQ ID NO: 32 - DNA, 24 bp, Artificial Sequence
  ERNI-133F, forward primer
ttgctcaagcccccaggaatgtca

SEQ ID NO: 33 - DNA, 22 bp, Artificial Sequence
  Puro-8R, reverse primer
cgaggcgcaccgtgggcttgta

SEQ ID NO: 34 - DNA, 18 bp, Artificial Sequence
  ALDH3A2-3 primer
agtggtcaccgggggagt

SEQ ID NO: 35 - DNA, 20 bp, Artificial Sequence
  ALDH3A2-4 primer
tcacagacacaatgggcagg

SEQ ID NO: 36 - DNA, 21 bp, Artificial Sequence
  Actin RT-1 primer
aacaccccagccatgtatgta

SEQ ID NO: 37 - DNA, 20 bp, Artificial Sequence
  Actin RT-2 primer
tttcattgtgctaggtgcca

SEQ ID NO: 38 - DNA, 20 bp, Artificial Sequence
  REAPER F1 primer
caccagaacaaagtgaacga

SEQ ID NO: 39 - DNA, 21 bp, Artificial Sequence
  REAPER F2 primer
tgtttgacaaaaaattgatgc

SEQ ID NO: 40 - DNA, 30 bp, Artificial Sequence
  ERNI-738 primer
atgcgtcgacgtggatgtttattaggaagc

SEQ ID NO: 41 - DNA, 25 bp, Artificial Sequence
  ERNI+83 primer
atgcgctagctggcagagaacccct

SEQ ID NO: 42 - DNA, 60 bp, Artificial Sequence
  Cre-C primer
ccgccggagatcttaatgcccaagaagaagaggaagctgtccaatttactgaccgtacac

SEQ ID NO: 43 - DNA, 31 bp, Artificial Sequence
  Cre-R1 primer
tcgaattcgaatcgccatcttccagcaggcg

SEQ ID NO: 44 - DNA, 24 bp, Artificial Sequence
  ERNI-37F primer
accacggcaacgggagaggcttat

SEQ ID NO: 45 - DNA, 20 bp, Artificial Sequence
  ERNI-342F primer
tgggcaaaggcagaggaatc

SEQ ID NO: 46 - DNA, 17 bp, Artificial Sequence
  Puro-83R primer
gcgtggcggggtagtcg

SEQ ID NO: 47 - DNA, 34 bp, Artificial Sequence
  loxP marker site
ataacttcgtatagcatacattatacgaagttat

SEQ ID NO: 48 - DNA, 43 bp, Bacteriophage phi-C31
  misc_feature: attp site
acgcccccaactgagagaactcaaaggttaccccagttggggc

SEQ ID NO: 49 - DNA, 43 bp, Artificial Sequence
  NotI forward primer
ttcttgcggccgcagggagccatagcctgctcccatcatgccc

SEQ ID NO: 50 - DNA, 25 bp, Artificial Sequence
  Nco1 reverse primer
agaggagcccaggccatggcggaat

SEQ ID NO: 51 - DNA, 22 bp, Artificial Sequence
  ApaL forward primer
agtgcagctgcagtgcacggta

SEQ ID NO: 52 - DNA, 35 bp, Artificial Sequence
  BglII reverse primer
ttcttagatctgtgacaagcagtctccggttaaca

(19) United States
(12) Patent Application Publication
(10) Pub. No.: US 2017/0223938 A1
(43) Pub. Date: Aug. 10, 2017

(54) TRANSGENIC CHICKENS WITH AN INACTIVATED ENDOGENOUS GENE LOCUS

(71) Applicant: Alexion Pharmaceuticals, Inc., New Haven, CT (US)

(72) Inventors: Marie-Cecile Van De Lavoir, San Francisco, CA (US); Philip A. Leighton, San Francisco, CA (US)

(21) Appl. No.: 15/358,664

(22) Filed: Nov. 22, 2016

Related U.S. Application Data
(63) Continuation of application No. 14/200,401, filed on Mar. 7, 2014, now abandoned.
Application No. 14/200,401 is a continuation of application No. 12/192,020, filed on Aug. 14, 2008, now abandoned.
(60) Provisional application No. 60/964,891, filed on Aug. 14, 2007.

(51) Int. Cl. A01K 67/027; C12N 15/85

(52) U.S. Cl. CPC A01K 67/0278; A01K 67/0273; A01K 67/0271; C12N 15/8509; A01K 2207/15; A01K 2217/072; A01K 2217/206; A01K 2227/30; A01K 2267/01; C12N 2015/8518

(57) ABSTRACT

The present invention is transgenic chickens obtained from long-term cultures of avian PGCs and techniques to produce and transgenic birds derived from prolonged PGC cultures. In some embodiments, these PGCs can be transfected with genetic constructs to modify the DNA of the PGC, specifically to introduce a transgene encoding an exogenous protein. When combined with a host avian embryo by known procedures, those modified PGCs are transmitted through the germline to yield transgenic offspring. This invention includes compositions comprising long-term cultures of PGCs and offspring derived from them that are genetically modified. The genetic modifications introduced into PGCs to achieve the gene inactivation may also include, but are not restricted to, random integrations of transgenes into the genome, transgenes inserted into the promoter region of genes, transgenes inserted into repetitive elements in the genome, site specific changes to the genome that are introduced using integrase, site specific changes to the genome introduced by homologous recombination, and conditional mutations introduced into the genome by excising DNA that is flanked by lox sites or other sequences that are substrates for site specific recombination.

### BACKGROUND OF THE INVENTION

Transgenic animals offer the potential for tremendous advances in the sustainable production of valuable pharmaceutical products, such as antibodies. However, the production of transgenic animals involves significant technical hurdles that have only been overcome for a few species. The ability to incorporate genetic modifications encoding exogenous proteins into the DNA of another species requires several distinct technologies that must be developed for each species. One approach to alter the genetic and physical characteristics of an animal is to introduce cells into recipient embryos of the animal. These cells have the ability to contribute to the tissue of an animal born from the recipient embryo and to contribute to the genome of a transgenic offspring of a resulting animal.

In certain cases, the cells can be engineered with a transgene that contains the DNA that encodes an exogenous product such as a protein or an antibody. The transgene contains the blueprint for the production of the protein and contains sufficient coding and regulatory elements to enable the expression of the protein in the tissue of the animal that is created from the insertion of the cells into a recipient embryo. In some circumstances, the expression is desired to be ubiquitous so that the expression occurs in all tissue types. However, in most circumstances where valuable proteins are desired, such as the collection of a valuable antibody, the expression must be limited to certain specific tissue types that facilitate collection of the expressed protein. For example, in cows, the expression of a protein in the milk enables the ready collection of the protein by simply collecting the milk of the cow and separating the exogenous protein. In chickens, the robust production of antibodies in the white of the egg also provides an attractive vehicle for the expression and collection of valuable proteins. Furthermore, where the tissue specific expression is specific to the oviduct of a chicken, the expression yields antibodies having certain specific desirable chemical properties that increase the therapeutic utility of the antibodies when used in the treatment of a human patient. Thus, one particularly attractive field of research and commercial development is genetically engineered chickens that selectively express antibodies in either egg white or egg yolk to facilitate isolation and collection of proteins with desirable chemical properties.

For the production of exogenous antibodies, avian biological systems offer many advantages including efficient farm cultivation, rapid growth, and economical production. Further, the avian egg offers an ideal biological design, both for massive synthesis of antibodies and ease of isolation and collection of product. Furthermore, as described below in the context of the present invention, advantages of the transgenic chicken expression system, compared for example to vertebrate, plant, or bacterial cell systems, are readily demonstrated and can be applied to produce uniquely advantageous chemical properties for large quantities of antibody product. The goal of creating a transgenic chicken has been sought by scientists for many, many years. Although the goal has been reached in other species, such as mice, cows, and pigs, transgenic chickens have not been created other than through the use of retroviral technology or direct injection technologies that suffer from inherent limitations on the size of a transgene that may be introduced into the DNA of the transgenic animal and/or lack of expression. In addition, viral vectors are not amenable to applications that require site specific changes to the genome such as those provided by homologous recombination.

Furthermore, in some circumstances, the animal's own endogenous genes could interfere with the production of valuable proteins resulting from introducing genetic constructs specially designed to express such proteins. Under such circumstances, the ideal solution would be to inactivate the animal's endogenous genes. Unfortunately, because of the unique challenges in genetic engineering in chickens, transgenic chickens having site-specific modifications resulting in the inactivation of an endogenous gene locus have not been described. Still further, the introduction of site specific gene inactivations produces an animal that is lacking in endogenous gene function, and such an animal can be bred with a different animal that has complementary specific genetic modifications introduced into its genome. For example, a family of animals lacking a specific gene could be established, and, through breeding, combined with an animal containing a specific gene for a human. In such a case, a population of animals would be created having both endogenous gene inactivations as well as introduced genomic modifications for the production of specific animal phenotypes or for the production of proteins encoded by the insertion of endogenous genes. No such animals currently exist because viral vectors do not permit site specific targeting of the endogenous genome nor the ability to select for integration events. Thus, viral vectors do not provide the mechanism through which an activation of an endogenous gene locus can be accomplished.

If a cell culture was sufficiently stable to allow large transgenes to become integrated into the genome of the cell, or to allow introduction of site specific changes to the genome, a transgene encoding tissue specific expression of any protein could be passed to a transgenic organism by several different techniques depending on the target cell and the specific construct used as the transgene. The same techniques can be used to perpetuate organisms having inactivated endogenous genes. Whole genomes can be transferred by cell hybridization, intact chromosomes by microcells, subchromosomal segments by chromosome mediated gene transfer, and DNA fragments in the kilobase range by DNA mediated gene transfer (Klobutcher, L. A. and F. H. Ruddle, Annu. Rev. Biochem., 50: 533-554, 1981). Intact chromosomes may be transferred by microcell-mediated chromosome transfer (MMCT) (Fournier, R. E. and F. H. Ruddle, Proc. Natl. Acad. Sci. U.S.A., 74: 319-323, 1977). The specific design of any such transgene carrying an exogenous gene or gene inactivation also must consider the content of the exogenous gene, the nature of any gene inactivation and the characteristics of the resulting phenotype in the animal.

Insertion of the transgenes that inactivate an endogenous locus or that enable tissue specific expression may threaten the pluripotency of the cells unless the transgenes are carefully designed. Thus, suitable cell lines must be both stable in culture and must maintain pluripotency when the cell is transfected with a genetic construct that is large and complex enough to either inactivate a gene or to contain all of the elements necessary for tissue specific and high-level expression where desired. In the resulting transgenic animal, the transgene may optionally be selectively expressed in specific individual tissue types in which the transgene is designed to be expressed. Depending on the genetic content of the transgene, the transgene may not be expressed in other tissues if the viability of the animal or the advantageous chemistry of the resulting protein is compromised.

Chicken primordial germ cells have been genetically modified using a retroviral vector within a few hours following isolation from Stage 11-15 embryos (Vick et al., (1993) Proc. R. Soc. Lond. B 251, 179-182). However, the resulting modification is randomly integrated and the size of the transgene is generally limited to less than about 15 kb, usually less than 10 kb and most commonly less 8 kb and site-specific changes to the genome cannot be created using this technology, nor can transferred cells be selected to identify site specific modifications to the exclusion of random integration. Stable genetic modifications requiring the insertion of greater than 15 kb of exogenous DNA into the genome of cultured avian PGCs have not been previously reported.

Any limitation on the size or site specificity of any DNA transgene or construct that may be stably introduced in a long-term PGC cell culture is a critical constraint on the ability to achieve valuable genetic modifications in the genome of PGCs in culture, and in turn, limits the types of genetic modifications that may be passed through the germline to offspring of the recipient embryo. For example, the introduction of an inactivation vector or an exogenous DNA sequence encoding a protein into the genome of a transgenic chicken is a highly desirable genetic modification. If a flock of such transgenic chickens could be created, large quantities of valuable proteins could be expressed in the chicken and collected in the egg. The avian egg offers an ideal repository for biologically active proteins and provides a convenient milieu from which proteins can be isolated. Avian animals are also attractive candidates for a broad variety of transgenic technologies. However, application of the full range of mammalian transgenic techniques to avian species has been unsuccessful due to the absence of a cultured cell population into which genetic modifications can be introduced and transmitted into the germline. In a recent paper, Sang et al. state: “It is unlikely that PGCs can be maintained in culture and proliferate for the extended period necessary to identify gene targeting events without losing their ability to migrate to the developing gonad after transfer.” Prospects for Transgenesis in the Chick. Mechanisms of Development, 121, 1179-1186, (2004). Therefore, to date, genetically transfected PGCs have not been created and the transmission to a mature living animal of a genetic modification introduced into an avian PGC has not been demonstrated.

Primordial germ cells (PGCs) are the precursors of sperm and eggs and are segregated from somatic tissues at an early stage of development in most animals. Pursuant to this invention, chicken PGCs are isolated, cultured and genetically modified while maintaining their commitment to the germline. In addition, PGCs are induced to differentiate into embryonic germ cells (EGCs), which resemble chicken embryonic stem cells (ESCs) in their commitment to somatic tissues. These PGCs commit to somatic tissues and the germline and provide a unique resource for genetic modification of the genome in chickens.

The production of transgenic animals especially mice has been important for the elucidation of mammalian gene function. The traditional approaches are random integration of the transgene into the genome or targeted insertion of transgenes into a specific locus by homologous recombination.

Random insertion of transgenes has two disadvantages. The first and major disadvantage is that many genes serve an essential function at various stages of development and elimination of transcription of these genes frequently causes embryonic mortality. Embryonic mortality can be obviated using site-specific recombinases such as Cre-loxP or Flp-FRT under the control of promoters that confer tissue specificity and developmentally regulated gene expression. In these cases, site-specific recombination is used to inactivate a gene in discrete cells and/or at discrete times during development within the context of an otherwise normal animal (termed conditional gene modification). For example the Cre-loxP system was used to specifically inactivate the insulin receptor gene in the p cells to create an insulin secretory defect similar to that in Type 2 Diabetes (Kulkarni et al. 1999 Tissue-specific knockout of the insulin receptor in pancreatic beta cells creates an insulin secretory defect similar to that in type 2 diabetes. Cell 96:329-39). Initially, it was shown that a null allele of DNA polymerase β is embryonic lethal when homozygous. To analyze its possible requirement for antigen receptor gene arrangement a conditional knockout approach was used to create a deficiency of DNA polymerase β in T-cells (Gu et al. 1994. Deletion of a DNA polymerase beta gene segment in T cells using cell type-specific gene targeting. Science 265:103-106).

Both random and targeted insertions of transgenes suffer from their inability to excise the positive selection cassette from the transgene in the transgenic animal. The presence of the selection cassette can cause a number of problems, such as disruption of gene expression at neighboring loci due to strong transcription regulatory elements frequently present in the selection cassettes (Lerner et al. 1993 CD3 zeta/eta/theta locus is colinear with and transcribed antisense to the gene encoding the transcription factor Oct-1. J Immunol. 151:3152-62; Ohno et al. 1994 Targeted disruption of the CD3 eta locus causes high lethality in mice: modulation of Oct-1 transcription on the opposite strand. EMBO J. 13:1157-65). The removal of a positive selection cassette can be achieved by a using a site-specific recombinase under the control of a tissue specific promoter.

Cre is a recombinase that catalyzes recombination between two loxP sites that are 34 base pair DNA elements. When two loxP sites are integrated into the genome in the same orientation, recombination catalyzed by Cre excises the intervening DNA. The loxP sites can be integrated into the transgene before it is randomly inserted into the genome or the loxP sites can be inserted into the genome at precise locations using targeting vectors. Following excision of intervening DNA, the flanking loxP sites are converted into a single loxP site. Mutant loxP sites are available that yield a product that is not well recognized following Cre excision. Flp recombinase, another member of the X intergrase superfamily of site-specific recombinases shares the same mechanism of DNA recombination with Cre recombinase. Similar to Cre, Flp recombinase recombines DNA at two defined 34 base pair target sites (FRT sites). Following excision of the intervening DNA, the flanking FRT sites are also converted into a single FRT site.

One of the uses of a conditional knockout is expression of lethal products in cells that are to be ablated in a particular tissue at a precise stage of development. For example, Grieshammer et al. (1998 Muscle-specific cell ablation conditional upon Cre-mediated DNA recombination in transgenic mice leads to massive spinal and cranial motoneuron loss. Dev Biol. 197:234-47) used the Cre-loxP system to express the Diphtheria toxin A fragment specifically in muscle cells to study skeletal muscle development in mice. Ligand-regulated forms of Cre also have been developed with the goal of adding temporal control of the Cre-loxP system to allow a precise induction of genetic changes in vitro or in vivo late in embryogenesis and/or in adult tissues.

Chromosomal rearrangements are a major cause of inherited disease and fetal loss, and have been associated with the progression and maintenance of cancer (Ramirez-Solis et al. 1995 Chromosome engineering in mice. Nature 378:720-4; Rabbitts et al. 2001 Mouse models of human chromosomal translocations and approaches to cancer therapy. Blood Cells Mol Dis. 27: 249-59). Chromosomal translocations often result in abnormal gene fusions and, consequently, tumor specific mRNAs and proteins are attractive targets for gene therapy. Thus, the ability to engineer chromosomal rearrangements with specific breakpoints by using the site-specific recombinase has been used to make mouse models of human disease. For example, translocations corresponding to the human rearrangements t (8:21)(q22; q22) and t (9:11)(p22q23) have been induced successfully in the mouse (Buchholz et al. 2000 Alteration of Cre recombinase site specificity by substrate-linked protein evolution. Nat Biotechnol. 19: 1047-52; Collins et al. 2000 Inter-chromosomal recombination of Mil and Af9 genes mediated by cre-loxP in mouse development. EMBO Rep.1: 127-32) in order to model acute leukemia. Random chromosome deletions can be generated by inserting loxP sites at random locations in the genome and then expressing Cre recombinase (Zhu et al. 2007. Efficient generation of random chromosome deletions, Biotechniques 42, 572-575).

Conditional gene modifications have been a powerful tool to manipulate gene expression during specification of cell lineages and analyses of cell fate has contributed to the understanding of normal development. The Cre-loxP system has been used to genetically activate lineage tracers in mice as for example in the determination of the adult fates of engrailed 2-expressing cells that originate in the midbrain-hindbrain constriction (Zinyk et al. 1998 Fate mapping of the mouse midbrain-hindbrain constriction using a site-specific recombination system. Curr Biol. 8: 665-8). This approach involved two mouse strains that were intercrossed. One Cre recombinase mouse, expressing Cre under the control of an engrailed-2 (En-2) genomic regulatory fragment that directs expression to the embryonic midbrain-hindbrain constriction region and an indicator/reporter mouse, harboring a transgene that “indicates” that recombination has occurred and provides a permanent record of this event by transforming it into a heritable lineage marker. The indicator line has a loxP-stop of transcription/translation-loxP-stop cassette driven by regulatory sequences from the widely expressed chick p actin gene. On crossing En2-Cre mice and the indicator mice the double transgenics carrying one copy of each transgene. Only cells that expressed Cre under the En2 regulatory element underwent recombination between the loxP of the reporter construct, excising the stop and permitting lacZ expression. As the Cre-mediated excision is cell heritable, the marked cells and all their progeny expressed lacZ at later stages even after Cre is no longer expressed. Thus, staining for LacZ in brains of adult double transgenic animals revealed the progeny of all cells that expressed Cre transiently during development in the midbrain-hindbrain constriction.

### SUMMARY OF INVENTION

This invention includes transgenic chickens and technologies enabling genetic engineering of transgenic birds, and the long-term culture of PGCs used to create transgenic chickens harboring inactivated endogenous loci resulting from the homologous integration of targeting constructs into primordial germ cells. These transgenic chickens have transgenes integrated into the genome of a chicken primordial germ cell by homologous recombination resulting in gene inactivation resulting from the deletion of at least a portion of an endogenous locus. The invention includes the transgene construct, stable cultures of primordial germ cells bearing the transgene, sometimes referred to as a knockout vector, a targeting vector, a knockout construct, or the like, wherein the transgene designed for endogenous gene inactivation is stably incorporated into the genome of a primordial germ cell maintained in culture for enough time to achieve the recombination event and select transfected cells.

The invention also includes primordial germ cells and the resulting transgenic chickens whose genome has been modified by inactivating an endogenous locus, including but not limited to the site specific deletion of a portion of a gene necessary for endogenous gene expression. For all of the foregoing embodiments, the invention also includes the resulting transgenic chickens produced from site specific modification of the endogenous genome. The invention also relates to antibodies produced in chickens having advantageous chemical properties that enhance their therapeutic utility in certain applications. Antibodies produced in chickens have a distinct pattern of chemical modifications compared to antibodies produced in vertebrate, plant, or bacterial cell systems such that when administered to a patient with the goal of binding a toxin to target tissue, such as tumors, the target tissue is treated with increased therapeutic efficacy. In one embodiment, long term cultures of PCGs are engineered with specially designed genetic constructs to introduce genetic modification into birds, including the insertion of transgenes that yield tissue specific expression of exogenous proteins. Either through engineering inactivated gene loci in the same pluripotent cells, or through engineering discrete populations of transgenic chickens bearing an inactivated endogenous loci, for subsequent breeding with birds having inserted transgenes to facilitate the expression of exogenous proteins, transgenic birds carrying a combination of exogenous DNA encoding the expression of a protein, combined with transgenic chickens having an inactivated endogenous locus, provide a uniquely advantageous population of animals expressing exogenous proteins.

Transgenic chickens having an inactivated endogenous locus also provide valuable animal models for the study of gene expression and for the selection of unique genetic functions that are not possible without the ability to inactivate a selected endogenous locus. Similarly, the inactivation of endogenous chicken loci may be performed at specific portions of the endogenous immunoglobulin locus including the V, D, or J regions to interrupt immunoglobulin gene rearrangement and to inactivate the endogenous antibody expression. As a result, one embodiment of the present invention includes a transgenic chicken substantially lacking endogenous immunoglobulin gene expression, and endogenous immunoglobulin protein production, resulting from the site specific gene modification at a selected portion of the endogenous chicken immunoglobulin locus. In a preferred embodiment, a transgene is constructed for the targeted inactivation of both the light chain and heavy chain encoding the endogenous immunoglobulin production. Transgenic birds of the invention may also express the transgene-derived antibody in the oviduct and the antibody is deposited in large quantities in the egg. In preferred embodiments, exogenous antibody proteins are encoded by human DNA sequences expressed in a background lacking endogenous antibody production such that native human antibodies are expressed in the chicken oviduct in the absence of endogenous avian antibody production thereby creating the ability to collect exclusively human antibodies from the egg.

The present invention includes populations of birds exhibiting tissue specific expression of antibodies, transgene constructs that enable exogenous antibody expression, isolated compositions of antibodies produced in chickens and having specially defined chemical properties, and related methods for creation of the birds, production of the antibodies and their therapeutic use in humans. The invention uses long term primordial cell cultures and special techniques to produce chimeric or transgenic birds derived from long term PGC cell cultures, wherein the genome of the PGCs have a stably integrated transgene expressing an exogenous protein such that progeny of the cultured cells contain the stably integrated transgene. When introduced to a host avian embryo, by the procedures described below, those modified donor cells produce birds that express the transgene into specific, selected somatic tissue of the resulting animals.

This invention also includes compositions of exogenous proteins expressed in transgenic chickens and having certain desirable chemical properties compared to vertebrate, plant, or bacterial cell systems. Specifically, these proteins, particularly antibodies, have reduced concentrations of fucose, galactose, N-acetyl neuraminic acid, N-glycolylneuraminic acid and elevated concentrations of mannose. Antibodies having some or all of these properties exhibit increased therapeutic utility when administered to a human. Specifically, these antibody compositions exhibit enhanced antibody-dependent cellular cytotoxicity (ADCC). Accordingly, the methods of the invention include using transgenic chickens to enhance the therapeutic utility, based on the ADCC effect, of compositions of antibodies by expressing them in a transgenic chicken.

The invention also includes transgenic chickens expressing exogenous antibody, having the advantageous chemistry defined herein, in the oviduct tissue such that exogenous antibody is concentrated in defined quantities in the egg white. In one preferred embodiment, the exogenous protein is a human sequence monoclonal antibody encoded by the transgene construct incorporated into the genome of a transgenic bird. The human monoclonal antibody encoding polynucleotide sequence is contained within a transgene that is specifically constructed for expression in the oviduct and which contains appropriate promoters and regulatory sequences to facilitate tissue specific expression.

This invention also relates to long-term cultures of avian primordial germ cells (PGCs) and several additional inventions enabled by the creation of a long-term culture where avian PGCs proliferate and where PGC cultures can be extended through multiple passages to extend the viability of the culture beyond 40 days, 60 days, 80 days, 100 days, or longer. The PGCs of the invention proliferate in long term cultures and produce germline chimeras when injected into recipient embryos.

The invention also relates to the introduction of genetic material into the genome of PGCs to obtain a desired outcome. In one embodiment, genetic constructs surrounded by HS4 elements are incorporated into PGCs of the invention to ensure the production of the transgene product. In another embodiment, the genetic modifications are executed using integrase to direct insertion of the construct into repetitive elements of the chicken genome. In another embodiment, DNA encoding a selectable marker is inserted into a region of the chicken genome to prevent production of the gene product.

Conditional mutations have been generated in chicken cells but unlike the mouse where transgenic lines expressing Cre have been made, lines of transgenic chickens expressing Cre recombinase under the control of ubiquitous, tissue specific or developmentally regulated promoters have not been made. In murine cells, transient expression of Cre recombinase (Araki et al. 1997 Efficiency of recombination by Cre transient expression in embryonic stem cells: comparison of various promoters. J Biochem 122:977-82) and a cell permeable Cre recombinase (Jo et al. 2001 Epigenetic regulation of gene structure and function with a cell-permeable Cre recombinase. Nat Biotechnol. 19:929-33) have been used to excise DNA between loxP sites. In the chicken DT40 cell line, however, transient expression of Cre recombinase was unable to remove DNA between loxP sites (Fukagawa et al. 1999 The chicken HPRT gene: a counter selectable marker for the DT40 cell line. Nucleic Acids Research 27, 19661969). Subsequently, the Cre transgene was incorporated into the genome of DT40 cells to achieve excision of DNA sequences between loxP and/or mutant loxP sites (Fukagawa et al. 1999 The chicken HPRT gene: a counter selectable marker for the DT40 cell line. Nucleic Acids Research 27, 1966-1969; Arakawa et al. 2001 Mutant loxP vectors for selectable marker recycle and conditional knock-outs BMC biotechnology 1, 7-14; Dhar et al. 2001 DNA repair studies: experimental evidence in support of chicken DT40 cell line as a unique model. J. Environ Pathol Toxicol Oncol 20, 273-83; Kanayama et al, 2005 Reversible switching of immunoglobulin hypermutation machinery in a chicken B cell line. Biochem. Biophys. Res. Commun. 327, 70-75).

The ability to make conditional mutations in the chicken would be advantageous. For example, it may be possible to create chickens that are substantially derived from embryonic stem cells using an apoptosis inducing gene under the control of a ubiquitous promoter that is silenced by the presence of a stop codon flanked by loxP sites. When this line of chickens is crossed to a line of birds that carries a gene encoding Cre recombinase under the control of a promoter that is expressed in the area pellucida, the embryo will not develop. If embryonic stem cells are injected into the embryo coincidentally with the expression of the apoptosis inducing gene, the embryo may be substantially derived from embryonic stem cells.

In another application, transgenes that contain sequences encoding selectable markers may be flanked by loxP sites. Transgenic birds carrying these transgenes may be crossed to birds expressing Cre recombinase under the control of a promoter that is expressed in the germline. Birds produced from this cross will hatch following excision of the selectable markers.

### DESCRIPTION OF THE FIGURES

FIG. 1A: PGCs maintained in culture for 54 days. Note that the cells are not attached and maintain a round morphology. Arrows indicate several dividing cells that are visible in this culture.

FIG. 1B: Long term PGC cell cultures are shown to be stable when maintained in culture for at least 136 days. These cells are cultured on a feeder layer of irradiated STO cells.

FIG. 2: Gene expression as determined by RT-PCR of the germ cell markers CVH (Vasa) and Dazl. Cells were in culture for 32 days. Lane 1 shows expression of both CVH and Dazl in an aliquot of PGCs. A second sample, in lane 2, did not have sufficient mRNA as determined by the absence of actin. CES cells were also analyzed; actin was expressed but the cES cells did not express CVH and Dazl was expressed only weakly.

FIG. 3: Western analysis of sample PGC cultures numbered #13 and #16 maintained in culture for 166 days. Testis was used as positive control and liver as a negative control. Rabbit anti-chicken CVH IgG was used as the primary antibody.

FIG. 4: Telomeric Repeat Amplification Protocol (TRAP) Assay. Different dilutions of cell extracts of 2 different PGC cell lines (numbered #13 & #16) maintained in culture for 146 days. The positive control consisted of the transformed human kidney cell line 293 and the negative control was lysis buffer only with no template added. In the PGC and positive control lanes, repeat sequences are visible indicating the presence of telomerase.

FIG. 5A: cEG cells derived from PGCs maintained in culture. Note the small cells, big nucleus (light grey) and pronounced nucleolus in both cell types.

FIG. 5B: Chicken embryonic stem cells. Note the small cells, big nucleus (light grey) and pronounced nucleolus in both cell types.

FIG. 6: Southern analysis of cx-neo transgene in a line of primordial germ cells (PGCs).

FIG. 7: FACS analysis of DT40 cells (negative control population), EG cells, ES cells and PGCs, stained with antibodies against chicken vasa homologue (CVH) and 1B3. The DT40, ES and EG cells were negative for both markers while a large majority of PGCs stained for both CVH and 1B3. The cell lines used were PGC 102; ES 439 and EG 455.

FIG. 8: Southern analysis of the HS4-β-actin-neo transgene in 2 lines of primordial germ cell PGCs.

FIG. 9: Southern analysis of the HS4 beta-actin-eGFP-beta-actin-puro transgene in primordial germ cell (PGC) line TP 103. The plasmid control DNA was linearized with Notl. An internal fragment was released by digesting the DNA with Kpnl. In both TP 103 and the plasmid, a fragment of the same size was released. Digestion of genomic DNA of TP 103 with Ncol, Mfel, and Sphl should reveal bands that are larger than the corresponding lanes of digeted plasmid DNA. No band is seen in the lane of Mfel digested TP 103 genomic DNA, which may be due to the band being too large. In the lanes representing the Ncol and Sphl digestions, fragments have been released in the TP 103 genomic DNA that are substantially larger than the fragments released in the plasmid DNA, indicating that the transgene is incorporated into the genome of the TP103 cell line.

FIG. 10: Karyotype of G-09 showing all chromosomes to be diploid. In one copy of GGA 2, the majority of the p arm is either missing or translocated to another chromosome. The other copy of CGA 2 is normal. The cells are ZZ (male).

FIG. 11: Section of testes, at 18 days of development, stained with DAPI. GFP positive germ cells are clearly visibly within the seminiferous tubules.

FIG. 12: The DAPI stained panel shows a section through a seminiferous tubule of an El 8 testis. GFP expressing cells are located within the seminiferous tubules and stain with the anti-CVH antibody.

FIG. 13: Transgenic offspring that have developed to between Stage X (EG&K) and Stage 34 (H&H) from a chimera carrying PGCs that are stably transfected with a β-actin-GFP transgene. All of the tissues revealed in these photopgraphs reveal expression of GFP.

FIG. 14. Tissues from a chimera carrying PGCs that are stably transfected with a β-actin-GFP transgene prepared for histological examination. The blue DAPI stain reveals the presence of nuclei and green fluoresence demonstrates that all of the tissues express the GFP transgene.

FIG. 15: Southern blot analysis showing that a clonally-derived, transfected PGC line can contribute to the germline in chimeric chickens and differentiate into EG cells. Upper panel: Genomic DNAs from PGCs transfected with the HS4 bactin-eGFP-bactin-puro construct, three embryos derived from a chimeric rooster made with the transfected PGCs and EG cells derived from the transfected PGCs, were giested with restriction enzymes for detecting internal (KpnY) and junction fragments (Ncol, AfllI) of the transgene insertion. The digested DNA was separated on a 0.7% agarose gel, blotted to nylon membrane, and probed with radiolabeled eGFP sequences. The sizes of the hybridizing fragments were identical in the PGCs, EG cells, and two embryos that showed green fluorescence (GFP+ embryos). A third, non-fluorescing embryo (WT embryo) showed no hybridization. Lower panel: a schematic of the construct is shown, with the locations of the restriction sites indicated, and the expected restriction fragment sizes shown below. There are two Kpn\ sites, resulting in a 5.3 kb fragment. Ncol and AftII cut within the construct once, and therefore the restriction fragments observed are junction fragments joining the construct with the flanking genomic DNA at the insertion site.

FIG. 16A. Diagrams of the random integration constructs used in this study. Two basic types of construct were used: selectable marker cassettes (drug-resistance markers and EGFP) driven by strong promoters, and similar constructs flanked by two sets of the HS4 insulator. The promoters used were mouse PGK, chicken β-actin, β-actin plus CMV enhancer (CAG) or ERNI. The constructs are as follows: a drug selectable market cassette consisting of a promoter driving expression of the neo or puro resistance gene (top line);

addition of the CAG-EGFP gene (second line); insulated drug selectable markers alone (third line); the same insulated selectable marker cassettes with the addition of an EGFP gene (fourth line); and the CAG-EGFP CAG-neo construction with loxP sites flanking the selectable markers and a proprietary gene of interest (box with asterisk). The constructs were linearized with Notl before transfection, resulting in the vector configuration shown.

FIG. 16B. Diagrams of the integrase constructs used in this study. On the left is the attB-containing plasmid in which an attB site was added to the HS4-β-actin-puro construct above. On the right is the plasmid used to express the integrase in cells from the CAG promoter. Both plasmids were transfected as circular DNAs.

FIG. 17. Alignment of attB with the attL sequences obtained from transfected PGCs. The junctions between the attB plasmid and genomic sequences in the PGC clones derived from integrase-mediated transfection are shown. On the top line is the wild type attB site, with the core TTG which is normally the recombination crossover point underlined. Below are shown the attL sequences from the integrase-mediated insertions in PGCs. To determine where the splice occurred between the attB on the plasmid and the pseudo attP site in the genome, the PGC sequences were compared to attB. In the PGC sequences, the attB sequences donated by the plasmid are in lower case, and the genomic pseudo attP sequences are in upper case and bold.

FIG. 18A-1 and FIG. 18A-2. Alignment of the P041-like repeats from PGC insertion sites with the P041 consensus sequence. The PGC flanking sequences from all of the clones inserted into P041-like repeats were aligned with each other and with the P041 consensus. The first 21 nucleotides are the attB sequence donated by the vector (as indicated above the alignment), followed by the genomic flanking sequence from each clone. Nucleotides shared by at least half of the sequences are boxed in black.

FIG. 18B. Alignment of attP with the P041 sequence. 100 bp of the attP site were aligned with 100 bp of P041, or roughly 2.5 copies of the 41 bp repeat. The core crossover TTG in attP is overlined.

FIG. 19. Targeting of the chicken IgL gene.

FIG. 20A. On the top line is a diagram of the targeting vector for the chicken IgL gene, IgL pK05. It is designed to replace the 2.3 kb J-C region of the IgL gene with a 3.1 kb HS4-ERNI-puro selectable marker flanked by the HS4 insulator. The two homology arms are 2.3 and 6.3 kb in length. At the 3′ end, a β-actin-EGFP gene allows for screening puro-resistant clones for green fluorescence to enrich for targeted clones. The dashed line at the end is the pKO vector backbone (Stratagene). On the middle line is a diagram of the wild type allele of the germline configuration of the IgL gene, with the single variable (V), joining (J) and constant (C) region genes. The restriction sites used for Southern analysis of targeted clones are shown (S, SacI; B, BstEll) and the wild type fragment sizes with double arrowheads shown below. On the lower line is the structure of the mutant allele in which the J and C regions have been deleted and replaced with HS4-ERNI-puro. The restriction map is shown, with the mutant fragment sizes shown below. The probes used in Southern analysis were both external to the targeting vector and their positions are shown. Scale bar=1 kb.

FIG. 20B. Southern blot analysis of 4 clones. Four puromycin-resistant clones were analyzed, two of which were non-green (clones 1 and 2) and two of which were green (clones 3 and 4). On the left panel, genomic DNA from the PGC clones was digested with Sacl and hybridized with probe A to analyze targeting on the 5′ side of the IgL gene. On the right panel, DNA was digested with BstEll and hybridized with probe B for targeting on the 3′ side of the IgL gene. Clone 2 showed the expected sized fragments for a heterozygous, targeted clone.

FIG. 21. PCR of ERNI-puro which is a marker for inactivation of the immunoglobulin light chain gene in semen from GO roosters made with PGCs carrying the IgL knock-out. 10 ng of genomic DNA prepared from semen samples was used in PCR for the ERNI-puro selectable marker present in the IgL knockout allele. As a control, primers for the endogenous chicken beta-actin gene were included.

As a positive control for the ERNI-puro PCR, the PGCs with the IgL knockout allele were used.

FIG. 22: ALDH3A2 expression in BN birds. The upper panel shows the RT-PCR for ALDH3A2 on RNA isolated from two homozygous BN birds (BN/BN), one heterozygous BN bird (BN/+) and one wild-type bird (+/+). In addition, a negative control of the RT-reaction (-RT-control), a positive genomic control and two negative controls for the PCR reaction (-control PCR) are shown. The 544 and 680 bp bands indicate the presence of mRNA of aldehyde dehydrogenase without and with an unspliced intron between exon 5 and 6, respectively. The 597 bp band in the lower panel confirms the presence of RNA in all samples. The RT-PCR showed that ADH was expressed in heterozygous BN birds but not in homozygous BN birds, indicating that insertion of the transgene stopped transcription of this gene.

FIG. 23. Sequence of the RT-PCR products from the wild-type allele at the aldehyde dehydrogenase 3 family member A2 transcripts. Products A and B are derived from a transcripts without and with an unspliced intron of 136 bp between exon 5 and 6, respectively.

FIGS. 24A and 24B: Chickens carrying the UbC-loxP-stop-loxP-Reaper transgene. FIG. 24A. Southern blot analysis of 3 UbC-loxP-stop-loxP-Reaper transgenic lines (6-03, 6-51 and 9-51). Genomic DNA samples from G1 birds and the UbC-loxP-stop-loxP-Reaper vector were digested with SpHI or Bell. The digested DNA was separated on a 0.7% agarose gel, blotted to nylon membrane and hybridized to a radiolabeled Reaper specific probe to identify junction fragments. The sizes of the hybridizing fragments were larger for the genomic DNAs than for the vector indicating that the transgenes were integrated.

FIG. 24B. Schematic representation of the UbC-loxP-stop-loxP-Reaper construct. The transgene consists of the UbC-promoter and the loxP-stop-loxP-Reaper transgene. A SV40 polyadenylation signal (SV40) and a Blasticidin resistance cassette (bsd) were inserted 3′ prime of UbC-loxP-stop-loxP-Reaper transgene. The construct was flanked by a 5′ and 3′ LTR. The locations of the restrictions sites are indicated and the expected restrictions sizes are shown.

FIGS. 25A and 25B: Chickens carrying the pLenti-ERNI-Cre transgene.

FIG. 25A. Southern blot analysis of the 8 ERNI-Cre lines. Genomic DNA samples were digested with Bglll. The digested DNA was separated on a 0.7% agarose gel, blotted to nylon membrane and probed with a radiolabeled Cre. The sizes of the hybridizing fragments were as expected 4.6 kb.

FIG. 25B. Schematic representation of the ERNI-Cre transgene. The transgene consists of ERNI promoter and the Cre transgene. A SV40 polyadenylation signal (SV40) and a Blasticidin resistance cassette (bsd) were inserted 3′ prime of ERNI-Cre transgene. The construct was flanked by a 5′ and 3′ LTR. The locations of the Bglll restrictions sites are indicated and the expected restrictions size is shown.

FIG. 26: FACS sorting of GFP positive and GFP negative cells from the Doc-2 cell line. The two classes of cells were generated by transfecting the Doc-2 cell line with a circular plasmid containing the ERNI-Cre transgene which expresses Cre recombinase in PGCs. The GFP negative cells are the result of excision of the sequence between the LoxP sites on the docking site vector that carries the CX-eGFP gene.

FIG. 27: Southern analysis of 2 chicks showing integration of the 10,652 bp transgene in the Doc-1 line of PGCs and chickens. Genomic DNA from the DOC1 PGC line (lanes marked P) and two chicks derived by breeding GO chimeras made with DOC 1 PGCs (lanes marked C1 and C2) was digested with either Bglll or EcoRI. The digests were fractionated on an agarose gel, transferred to nylon membrane and hybridized to radiolabeled EGFP sequences. This analysis detects junction fragments containing the docking site vector joined to the flanking genomic sequences at the site of integration of the vector. The size of these junction fragments varies depending on the site of integration and is diagnostic for each transgene insertion event in PGCs. In this case, the Bglll fragment is approximately 12 kb, and the EcoRI fragment greater than 12 kb. The fragment sizes are identical in PGCs and the chicks derived from them, showing that these chicks were derived from PGCs carrying the DOC1 insertion.

FIGS. 28A and 28B: Southern blot assay for Cre-mediated recombination of Reaper transgene by 10 different lines of pLenti-ERNI-Cre transgenic chickens.

FIG. 28A. Southern blot analysis on DNA isolated from brain (b) and muscle (m) from double transgenic embryos carrying one copy of the Cre transgene and one copy of the loxP transgene. Genomic DNA was digested with Sacl. The digested DNA was separated on a 0.7% agarose gel, blotted to nylon membrane and hybridized to a probe consisting of the Reaper gene and portions of the Lentiviral vector backbone (the blasticidin gene and SV40 sequences). This probe hybridizes equally to both full-length and recombined loxP-Reaper transgenes. The ratio of the band intensities of the full-length (non-recombined) to recombined transgenes represents the activity of the Cre lines.

FIG. 28B. Schematic presentation of the Cre-mediated recombination of the loxP-Reaper transgene. The full-length loxP-Reaper transgene contains a 1.4 kb sequence, called a stop cassette, flanked by loxP sites in the same orientation. Recombination between the two loxP sites results in excision of the 1.4 kb intervening sequence from the chromosome, leaving behind a single loxP site. After excision, the recombined loxP-Reaper transgene is reduced in size by 1.4 kb. The locations of the probes and of the SacI restrictions sites are indicated and the expected restrictions sizes are shown.

FIGS. 29A and 29B: Recombination of three different Reaper loxP cassette transgenes (603, 6-51 and 9-51) by the Cre4 line.

FIG. 29A: Southern blot analysis of transgenic embryos carrying only one copy of the loxP-Reaper transgene (R) or double transgenic embryos carrying one copy of the Cre4 transgene and one copy of the loxP-Reaper transgene (C+R) for 3 different loxP-Reaper lines (6-03, 6-51 and 9-51). Genomic DNA was digested with SacI. The digested DNA was separated on a 0.7% agarose gel, blotted to nylon membrane and hybridized to a radiolabeled probe consisting of the Reaper gene and portions of the Lentiviral vector backbone (the blasticidin gene and SV40 sequences). The sizes of the hybridizing fragments were as expected 2.8 kb for the full-length (unrecombined) loxP-Reaper fragment and 1.4 kb for the recombined loxP-Reaper fragment.

FIG. 29B: Schematic presentation of the Cre-mediated recombination of the loxP-Reaper transgene. The full-length loxP-Reaper transgene contains a 1.4 kb sequence, called a STOP cassette, flanked by loxP sites in the same orientation. Recombination between the two loxP sites results in excision of the 1.4 kb intervening sequence from the chromosome, leaving behind a single loxP site. After excision, the recombined loxP-Reaper transgene is reduced in size by 1.4 kb. The locations of the probes and of the SacI restrictions sites are indicated and the expected restrictions sizes are shown.

FIG. 30: Southern analysis of the un-excised GFP positive Doc 2 cell line (Lane 1) and Doc 2 cells from which the cx-GFP-cx-neo sequences have been deleted (Lane 2). The cells were sorted by FACS analysis for expression of green fluorescence. Genomic DNA from the two populations of cells was prepared and digested with Hindlll restriction enzyme and the DNA was hybridized with radiolabeled sequences from the puromycin resistance gene. A predicted fragment of 5521 bp was present in GFP positive (non-excised cells) cells and a predicted fragment of 1262 bp was present in the excised cells. This result indicates that Cre-lox recombination results in deletion of the CX-EGFP-CX-neo sequences lying between the two loxP sites in the docking site construct integrated in DOC2 cells.

FIG. 31: Diagram of the IgL pK05B targeting vector. The top line indicates the construction of the targeting vec IgL pK05B. This line shows the vector construction for the 5′ homology region including loxP and attP sites, as well as the 3′ homology region. The second line shows the relationship of the targeting vector to the chicken wild type IgL allele. The third line shows the mutant allele created by the J and C genes deletion or gene disruption.

FIG. 32: Southern blot analysis showing the IgL locus deletion in the KO-07 knockout clone. Left hand panel: The hybridization obtained for the 5′ homology region when DNA from the five clonal PGC lines transfected with IgL pK05B was digested with SacI and probed with a 0.5 kb Sacl-BstEII fragment. Wild type IgL locus is approximately 1Okb and the mutant fragment with the targeted deletion is approximately 4 kb. Right hand panel: The hybridization obtained for the 3′ homology region with DNA from the same five clones. The genomic DNA was digested with BstEII and hybridized with a 3′ 1.7 kb Nsil-Mfel fragment, which is also external to the targeting vector.

FIG. 33: Southern blot analysis showing IgL knockout was transmitted to 5 of 7 chicken embryos (embryos 2, 3, 4, 6 and 7). Embryos 1 and 5 were wild type embryos that inherited the wild type IgL allele from the heterozygous, targeted KO-07 knockout PGCs.

### DETAILED DESCRIPTION OF INVENTION

As used herein, the terms chicken embryonic stem (cES) cells mean cells exhibiting an ES cell morphology and which contribute to somatic tissue in a recipient embryo derived from the area pellucida of Stage X (E-G&K) embryos (the approximate equivalent of the mouse blastocyst). CES cells share several in vitro characteristics of mouse ES cells such as being SSEA-1+, EMA-1+ and telomerase+. ES cells have the capacity to colonize all of the somatic tissues.

As used herein, the terms primordial germ cells (PGCs) mean cells exhibiting a PGC morphology and which contribute exclusively to the germline in recipient embryos, PGCs may be derived from whole blood taken from Stage 12-17 (H&H) embryos. A PGC phenotype may be established by: (1) the germline specific genes CVH and Dazl are strongly transcribed in this cell line, (2) the cells strongly express the CVH protein, (3) the cells do not contribute to somatic tissues when injected into a Stage X nor a Stage 12-17 (H&H) recipient embryo, (4) the cells give rise to EG cells (see below), or (5) the cells transmit the PGC genotype through the germline when injected into Stage 12-17 (H&H) embryos (Tajima et al. (1993) Theriogenology 40, 509-519; Naito et al., (1994) Mol. Reprod. Dev., 39, 153-161; Naito et al, (1999) J Reprod. Fert. 117, 291-298).

As used herein, the term chicken embryonic germ (cEG) cells means cells derived from PGCs which are analogous in function to murine EG cells. The morphology of cEG cells is similar to that of cES cells and cEG cells contribute to somatic tissues when injected into a Stage X (E-G&K) recipient.

As used herein, the term transgenic means an animal that encodes a transgene in its somatic and germ cells and is capable of passing the traits conferred by the transgene to its progeny. The term transgenic also means an animal that contains a site selected, specific gene inactivation in the endogenous locus, including but not limited to the deletion of a finite gene segment in the endogenous locus, by use of a transgene or targeting construct that integrates into the genome of the primordial germ cell that results in gene inactivation through a gene literal deletion, a functional disruption, insertion of a stop codon, or non-sense sequences, attP site, or other artifact that yield a functionally inactivation of the locus through site specific gene modification. Because the existing retroviral technologies do not allow for site specific modification or the selection of transformed cells, the ability to sustain long-term cultures of PGC cells and to engineer site specific genetic modifications, such as gene inactivations, the term transgenic excludes retroviral systems.

But include animals bearing a site specific gene alteration that changes the function of a selected gene and yields a desired phenotype from the gene modification. These transgenes and the animals derived from them are commonly referred to as “knock-ins”. The transgenes may insert a deletion of endogenous DNA of at least 1Otcb, preferably 10-25′ kb, or more depending on the size and organization of the gene selected for targeting. In a preferred embodiment, the transgenic avian lacks any endogenous gene corresponding to the endogenous gene target for total or partial deletion or other function disruption.

Although the examples herein are described for chickens, other avian species such as quails, turkey, pheasant, and others can be substituted for chickens without undue experimentation and with a reasonable expectation for successful implementation of the methods disclosed here.

By inserting DNA constructs designed for tissue specific expression into ES cells in culture, chickens have been created that express valuable pharmaceutical products, such as monoclonal antibodies, in their egg whites. See PCT US03/25270 WO 04/015123 Zhu et al. A critical enabling technology for such animals is the creation and maintenance of truly long-term ES cell cultures that remain viable long enough for the genotype of the cloned cells to be engineered in culture.

Unlike ES cells, however, primordial germ cells (PGCs) have only been cultured on a short-term basis. Once the length in culture extends beyond a short number of days, these cells lose the ability to contribute exclusively to the germline. Typically, PGCs maintained in culture using current culture techniques do not proliferate and multiply. In the absence of robust growth, the cultures are “terminal” and cannot be maintained indefinitely. Over time, these terminal cell cultures are degraded and the cells lose their unique PGC morphology and revert to embryonic germ (EG) cells. Embryonic germ cells acquire a different morphology from PGCs, lose their restriction to the germline, and gain the ability to contribute to somatic tissues when injected into early stages of embryonic development. To introduce a predetermined genotype into the germline of a recipient embryo, thereby enabling the animal to pass the desired genotype on to future generations, PGCs are uniquely attractive because they are known to be the progenitors of sperm and eggs.

Long-term cultures of PGCs, with or without gene inactivating or insertions of exogenous DNA, provide several important advantages, such as sustaining valuable genetic characteristics of important chicken breeding lines that are relied upon in the poultry and egg production industries. Currently, extraordinary measures are undertaken to prevent valuable breeding lines from being lost through accident or disease. These measures require maintaining large numbers of members of a line as breeding stock and duplicating these stocks at multiple locations throughout the world. Maintaining large numbers of valuable animals in reserve is also necessary because preserving genetic diversity within a breeding line is also important. By preserving the genetic characteristics of valuable breeding lines in PGC cell cultures rather than in live reserve stocks, the expense of large scale reserve breeding populations is avoided. Long term cultures of PGCs are described in van de Lavoir, M-C, Diamond, J, Leighton, P, Heyer, B, Bradshaw, R, Mather-Love, C, Kerchner, A, Hooi, L, Gessaro, T, Swanberg, S, Delany, M, and Etches, R. J. (2006). Germline transmission of genetically modified primordial germ cells. Nature 441, 766-769. Producing genetically engineered chickens using PGCs requires introducing genetic modifications into the genotype of the PGCs, isolating the rare cells in which the genetic modification has occurred and expanding the population of genetically modified cells for analysis and introduction into recipient embryos to form GO chimeras. Techniques for a wide variety of genetic manipulations for targeting cells in culture are well known. However, one main difficulty is that to alter the genotype of PGCs in culture, the culture must remain viable for a length of time adequate to introduce the genetic modifications and to select successfully transformed cells, and while the transfected cells grow and proliferate in culture. Successfully transformed cells that are capable of proliferating are distinguished by their ability to generate large numbers of cells (e.g. 10⁴ to 10⁷ cells) within several days to several weeks following clonal or nearly clonal derivation. The founder cells will be the rare cells that carry the genetic modification that is desired. Typically, these cells are generated in culture at frequencies of 10-⁴ to 10-⁷ following the application of technologies for genetic modification that are well known, (e.g. lipofection or electroporation). Therefore, successful production of PGCs in culture requires passaging the cells to provide space and nutrients for the cells to proliferate and generate a sufficient number of cells to allow selection of the rare, genetically-modified cells in culture.

To provide such populations, the culture conditions must be sufficiently robust to allow the cells to grow from an individual genetically modified cell into a colony of 10⁴ to 10⁷ cells to be used for genetic analysis in vitro and for the production of chimeras. These engineered PGCs would contribute exclusively to the nascent population of spermatogonia or oogonia (i.e., the sperm and eggs) in the resulting animals upon maturity. In such a resulting animal, the entirety of the somatic tissue would be derived from the recipient embryo and the germline would contain contributions from both the donor cells and the recipient embryos. Because of the mixed contribution to the germline, these animals are known as “germline chimeras.” Depending on the extent of chimerism, the offspring of germline chimeras will be derived either from the donor cell or from the recipient embryo.

The germline in chickens is initiated as cells from the epiblast of a Stage X (E-G & K) embryo ingress into the nascent hypoblast (Kagami et al, (1997) Mol Reprod Dev 48, 501-510; Petitte, (2002) J Poultry Sci 39, 205-228). As the hypoblast progresses anteriorly, the pre-primordial germ cells are swept forward into the germinal crescent where they can be identified as large glycogen laden cells. The earliest identification of cells in the germline by these morphological criteria is approximately 8 hours after the beginning of incubation (Stage 4 using the staging system established by Hamburger and Hamilton, (1951) J Morph 88, 49-92). The primordial germ cells reside in the germinal crescent from Stage 4 (H&H) until they migrate through the vasculature during Stage 12-17 (H&H). At this time, the primordial germ cells are a small population of about 200 cells. From the vasculature, the primordial germ cells migrate into the genital ridge and are incorporated into the ovary or testes as the gonad differentiates (Swift, (1914) Am. J. Anat. 15, 483-516; Meyer, (1964) Dev. Biol. 10,154-190; Fujimoto et al. (1976) Anat. Rec. 185,139-154).

In all species that have been examined to date, primordial germ cells have not proliferated in culture for long periods without differentiating into EG cells. Long periods in culture are required in order to produce a sufficient number of cells to introduce a genetic modifications or inactivations by conventional electroporation or lipofection protocols. Typically, these protocols require 10⁵ to 10⁷ cells and therefore, production of these cells from a single precursor requires 17 to 24 doublings assuming that all cell divisions are (1) synchronous and (2) produce two viable daughter cells. The introduction of a genetic modification into the genome of a cell is a rare event, typically occurring in one in 1×10⁴ to 1×10⁶ cells. Following genetic modification, the cells must be able to establish a colony from the single cell that carries and/or expresses the genetic modification. The colony must be able to expand into a population of 10⁵ to 10⁷ cells that can be analyzed by PCR or Southern analysis to evaluate the fidelity of the transgene and provide a sufficient number of cells that are then injected into recipient Stage 13-15 (H&H) embryos. Therefore another 17 to 24 cell divisions are required to produce the populations of cells and in total 34 to 58 doublings are required to produce the population of genetically modified cells. Assuming that the cell cycle is 24 hours, a minimum of 34 days and in general 58 days in culture are required to produce genetically modified primordial germ cells for injection into Stage 13-15 (H&H) recipient embryos. The injected cells must then be able to colonize the germline, form functional gametes and develop into a new individual post fertilization.

The PGCs maintained in the culture described herein maintain a characteristic PGC morphology while maintained in culture. The PGC morphology may be observed by direct observation, and the growth of cells in culture is assessed by common techniques to ensure that the cells proliferate in culture. Cell cultures that proliferate are defined as non-terminal and are observed to have a greater number of cells in culture at the latter of 2 distinct time points. The PGCs in the culture of the invention may have 1×10⁵ or more cells in any particular culture and this number may be observed to increase over time. Accordingly, the invention includes a proliferating PGC culture that contains a larger number of cells after a period of days, weeks, or months compared to an earlier time point in the life of the culture. Ideally, the culture contains at least 1×10⁵ cells and may be observed to have a higher number after any length of time growing in culture. Furthermore, the PGCs may be observed to be the dominant species in the culture such that, when considering the minimal contribution made by non-chicken feeder cells, the proliferating component of the cell culture consists essentially of chicken primordial germ cells, to the substantial exclusion of other chicken-derived cells.

The culture also manifests the characteristic of allowing proliferation by passage such that samples or aliquots of cells from an existing culture can be separated and will exhibit robust growth when placed in new culture media. By definition, the ability to passage a cell culture indicates that the cell culture is growing and proliferating and is non-terminal. Furthermore, the cells of the invention demonstrate the ability to create germline chimeras after several passages and maintain a PGC morphology. As described herein, this proliferation is an essential feature of any cell culture suitable for stable integration of exogenous DNA sequences.

PGCs can be obtained by any known technique and grown in the culture conditions described herein. However, it is preferred that whole blood is removed from a stage 15 embryo and is placed directly in the culture media described below. This approach differs from other approaches described in the literature wherein PGCs are subjected to processing and separation steps prior to being placed in culture. Robust differential growth between PGCs and other cells from whole blood that may initially coexist in the medium provides the large populations of PGCs in culture described here. Accordingly, PGCs derived directly from whole blood are grown in culture into large cell concentrations, can go through an unlimited number of passages, and exhibit robust growth and proliferation such that the PGCs in culture are essentially the only cells growing and proliferating.

One aspect of the present invention is the creation of large numbers, including greater than 3, greater than 4, greater than 5, 10, 15 and 20 germline chimeric transgenic animals all having genetically identical PGC-derived cells in their germline. Another aspect of the invention is the creation of a population of germline chimeras having genetically identical PGC-derived cells in their germline that have, within the population, age differentials that reflect the use of the same long-term cell culture to create germline chimeras. The age differentials exceed the currently available ability to culture primordial germ cells over time and are as high as 190 days without freezing. Accordingly, the present invention includes two or more germline chimeras having identical PGC-derived cells in their germline that differ in age by more than 40 days, 60 days, 80 days, 100 days, 190 days, etc, or any other integral value therein—without freezing the cells. The invention also includes the existence of sexually mature germline chimeras having genetically identical PGC-derived cells in their germline, together with the existence of a non-terminal PGC culture used to create these germline chimeras and from which additional germline chimeras can be created.

Because the PGCs can be maintained in culture in a manner that is extremely stable, the cells can also be cryo-preserved and thawed to create a long-term storage methodology for creating germline chimeras having a capability to produce offspring defined by the phenotype of the PGCs maintained in culture.

The capability to produce large numbers of germline chimeras also provides the ability to pass the PGC-derived genotype through to offspring of the germline chimera. Accordingly, the present invention includes both populations of germline chimeras having genetically identical PGC-derived cells having an inactivated endogenous gene locus in the germline, but also offspring of the germline chimeras whose genotype and phenotype is entirely determined by the genotype of the PGCs grown in culture. Incorporation of a PGC-derived knockout phenotype in the germline has been observed. Thus, the invention includes the offspring of a germline chimera created by germline transmission of a genotype of a primordial germ cell comprising an inactivated endogenous locus. Accordingly, the invention includes each of the existence of a primordial germ cell culture containing PGCs comprised of a site specific gene inactivation, a germline chimera having the same primordial germ cells as part of its germline, and an offspring of the germline chimera having the knockout genotype and phenotype.

The ratio of donor-derived and recipient-derived PGCs in a recipient embryo can be altered to favor colonization of the germline in PGC-derived chimeras. In developing chicken and quail embryos, exposure to busulfan either greatly reduces or eliminates the population of primordial germ cells as they migrate from the germinal crescent to the gonadal ridge (Reynaud (1977a) Bull Soc. Zool. Francaise 102, 417-429; Reynaud (1981) Arch Anat. Micro. Morph. Exp. 70, 251-258; Aige-Gil and Simkiss (1991) Res. Vet. Sci. 50, 139-144). When busulfan is injected into the yolk after 24 to 30 hours of incubation and primordial germ cells are re-introduced into the vasculature after 50 to 55 hours of incubation, the germline is repopulated with donor-derived primordial germ cells and subsequently, donor derived gametes are produced (Vick et al. (1993) J. Reprod. Fert. 98, 637-641; Bresler et al. (1994) Brit. Poultry Sci. 35 241-247).

Methods of the invention include: obtaining PGCs from a chicken, such as from the whole blood of a stage 15 embryo, placing the PGCs in culture, engineering the inactivation of an endogenous gene locus, proliferating the engineered PGCs to increase their number and enabling a number of passages, creating germline chimeras from long-term cultures of the engineered PGCs, and obtaining offspring of the germline chimeras having a genotype and phenotype that exhibits the gene inactivation engineered in PGCs. The methods of the invention also include inserting a gene inactivation or gene “knockout” into a population of PGCs in culture to create stably transfected PGCs harboring an inactivated or functionally disrupted endogenous locus, selecting cells from this population that carry stably integrated transgenes, injecting the genetically modified cells carrying the stably integrated transgenes into a recipient embryo, developing the embryo into a germline chimera containing the inactivated locus in the germline, raising the germline chimera to sexual maturity and breeding the germline chimera to obtain transgenic offspring wherein the gene inactivation is derived from the cultured PGC. The genetic modifications introduced into PGCs to achieve the gene inactivation may include, but are not restricted to, random integrations of transgenes into the genome, transgenes inserted into the promoter region of genes, transgenes inserted into repetitive elements in the genome, site specific changes to the genome that are introduced using integrase, site specific changes to the genome introduced by homologous recombination, and conditional mutations introduced into the genome by excising DNA that is flanked by lox sites or other sequences that are substrates for site specific recombination

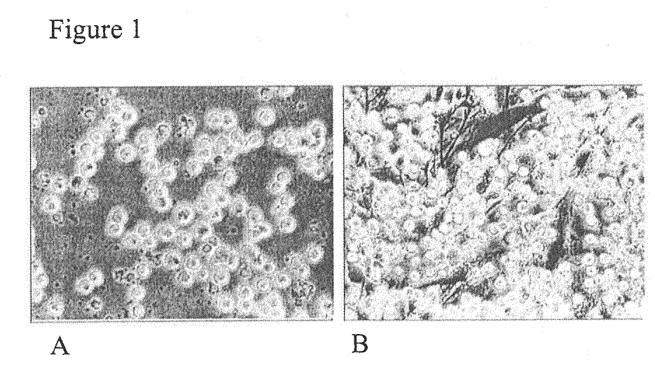

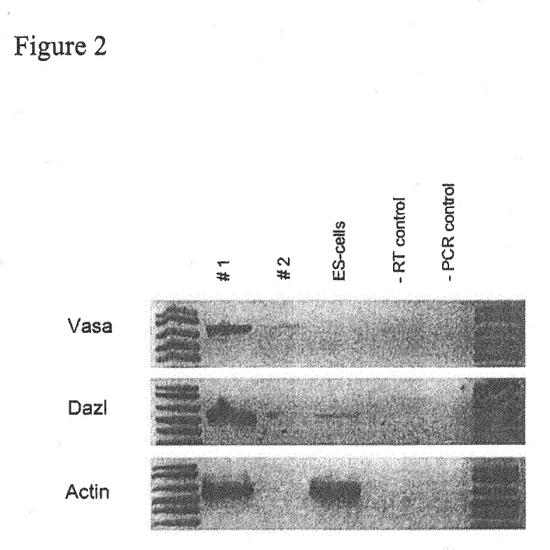

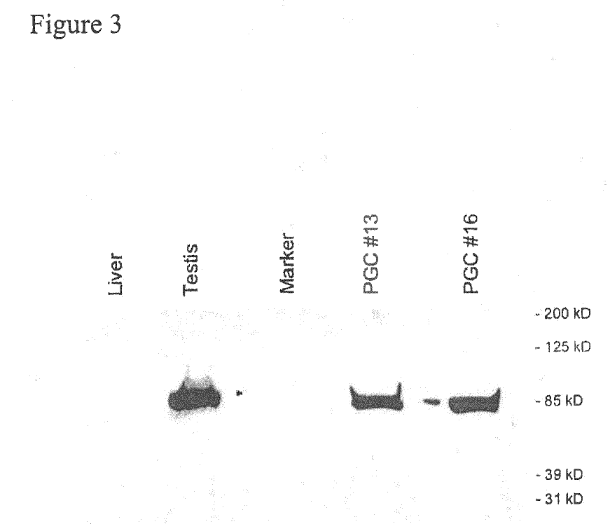

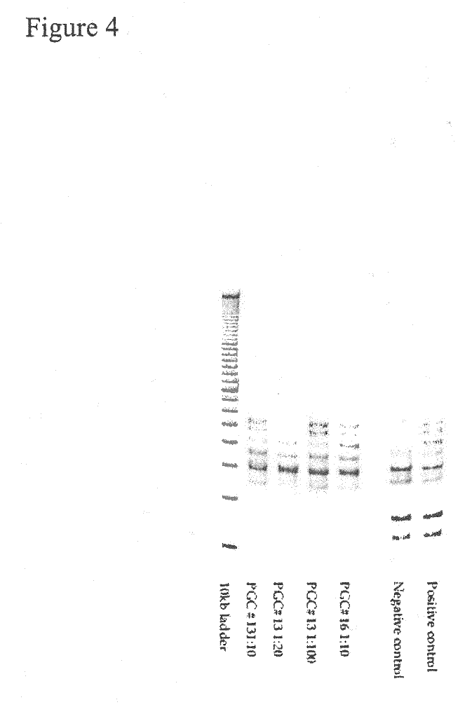

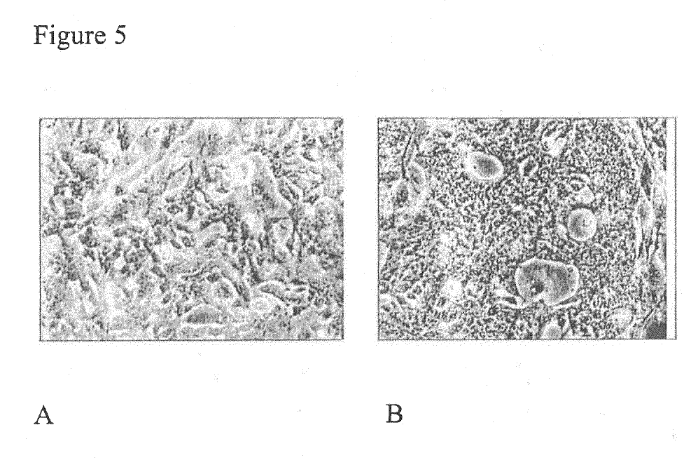

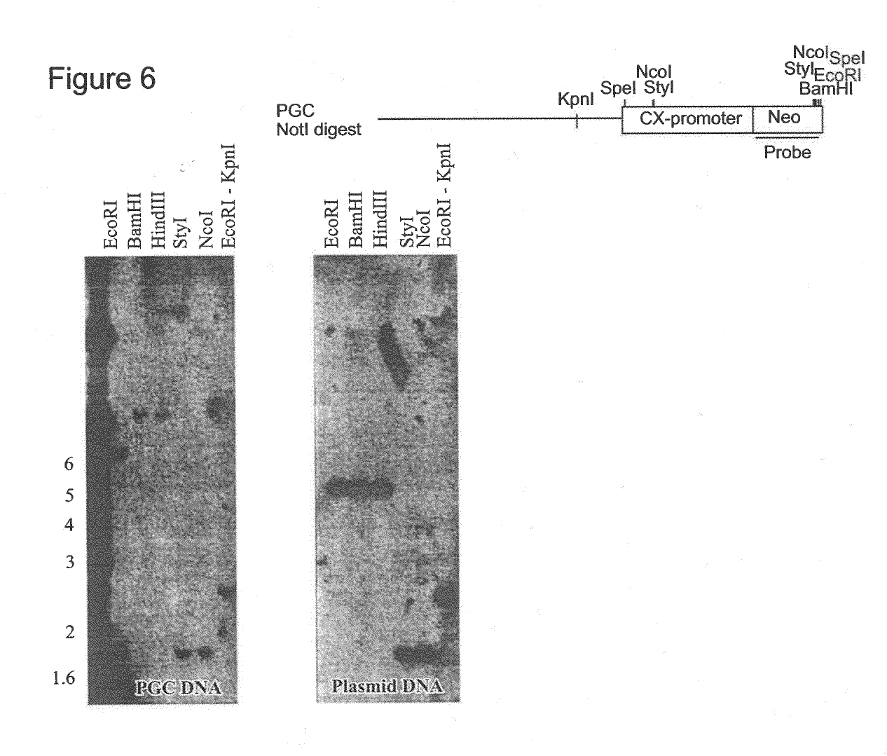

As described below, pursuant to this invention, chicken PGC cell lines have been derived from blood taken from Stage 14-16 (H&H) embryos that have a large, round morphology (FIG. 1). These cells are confirmed to be chicken PGCs by their morphology after long term culturing and their ability to yield PGC-derived offspring. In addition, the PGC cultures express the germline-specific genes Dazl and CVH (FIG. 2) and the CVH protein is produced by the cells in culture (FIG. 3). PGCs in culture also express telomerase (FIG. 4) indicating an immortal phenotype. Furthermore, PGCs give rise to embryonic germ (EG) cells in the appropriate culture conditions (FIG. 5). By analogy, mouse and human PGCs will give rise to EG cells when treated in an analogous fashion. Mouse EG cells will contribute to somatic tissues and chicken EG cells also contribute to somatic tissues as indicated by black feather pigmentation in chimeras. Chicken PGCs have been genetically modified as indicated by Southern analysis (FIG. 6). In this embodiment, the CX promoter is stably integrated into the genome of a PGC and is used to facilitate expression of the gene encoding aminoglycoside phosphotransferase (APH) which is also integrated into the genome of a PGC and is used to confer resistance to neomycin added to culture media in order to select PGCs that have been genetically modified.

### EXAMPLE 1

### Derivation of Cultures of Chicken PGCs

Two to five μL of blood taken from the sinus terminalis of Stage 14-17 (H&H) embryos were incubated in 96 well plates in a medium containing Stem Cell Factor (SCF; 6 ng/ml or 60 ng/ml), human recombinant Fibroblast Growth Factor (hrFGF; 4 ng/ml or 40 ng/ml), 10% fetal bovine serum, and 80% KO-DMEM conditioned medium. Preferably one to three μL was taken from the vasculature of a stage 15-16 (H&H) embryo. The wells of the 96-well plates was seeded with irradiated STO cells at a concentration of 3×10⁴ cells/cm².

KO-DMEM conditioned media were prepared by growing BRL cells to confluency in DMEM supplemented with 10% fetal bovine serum, 1% pen/strep; 2 mM glutamine, ImM pyruvate, IX nucleosides, IX non-essential amino acids and 0.1 mM B-mercaptoethanol and containing 5% fetal bovine serum for three days. After 24 h, the medium was removed and a new batch of medium was conditioned for three days. This was repeated a third time and the three batches were combined to make the PGC culture medium.

After approximately 180 days in culture, one line of PGCs was grown in media comprised of 40% KO-DMEM conditioned media, 7.5% fetal bovine serum and 2.5% chicken serum. Under these conditions, the doubling time of the PGCs was approximately 24-36 hours.

When the culture was initiated, the predominant cell type was fetal red blood cells. Within three weeks, the predominant cell type was that of a PGC. Two PGC cell lines were derived from 18 cultures that were initiated from individual embryos.

A line of PGCs has been in culture for over 9 months, maintain a round morphology, and remain unattached (FIGS. 1A & 1B). PGCs have been successfully thawed after cryopreservation in CO₂ independent medium containing 10% serum and 10% DMSO.

### EXAMPLE 2

### Cultured PGCs Express CVH and Dazl

Expression of CVH, which is the chicken homologue of the germline specific gene VASA in Drosophila, is restricted to cells within the germline of chickens and is expressed by approximately 200 cells in the germinal crescent. (Tsunekawa, N, Naito, M, Sakai, Y, Nishida, T. & Noce, T. Isolation of chicken vasa homolog gene and tracing the origin of primordial germ cells. Development 127, 2741-50. (2000). CVH expression is required for proper function of the germline in males; loss of CVH function causes infertility in male mice. (Tanaka, S. S. et al. The mouse homolog of Drosophila Vasa is required for the development of male germ cells. Genes Dev 14, 841-53. (2000). The expression of Dazl is restricted to the germline in frogs (Houston, D. W. & King, M. L. A critical role for Xdazl, a germ plasm-localized RNA, in the differentiation of primordial germ cells in Xenopus. Development 127, 447-56, 2000), axolotl (Johnson, A. D, Bachvarova, R. F, Drum, M. & Masi, T. Expression of axolotl DAZL RNA, a marker of germ plasm: widespread maternal RNA and onset of expression in germ cells approaching the gonad. Dev Biol 234, 402-15, 2001), mice (Schrans-Stassen, B. H, Saunders, P. T, Cooke, H. J. & de Rooij, D. G. Nature of the spermatogenic arrest in Dazl −/− mice. Biol Reprod 65, 771-776, 2001), rat (Hamra, F. K. et al. Production of transgenic rats by lentiviral transduction of male germ- line stem cells. Proc Natl Acad Sci USA 99, 14931-6, 2002), and human (Lifschitz-Mercer, B. et al. Absence of RBM expression as a marker of intratubular (in situ) germ cell neoplasia of the testis. Hum Pathol 31, 1116-1120, 2000). Deletion of Dazl led to spermatogenic defects in transgenic mice (Reijo, R. et al. Diverse spermatogenic defects in humans caused by Y chromosome deletions encompassing a novel RNA-binding protein gene. Nat Genet 10, 383-93, 1995).

After 32 days, PGCs were washed with PBS, pelleted and mRNA was isolated from the tissue samples with the Oligotex Direct mRNA kit (Qiagen). cDNA was then synthesized from 9 ul of mRNA using the Superscript RT-PCR System for First-Strand cDNA synthesis

(Invitrogen). Two u.1 of cDNA was used in the subsequent PCR reaction. Primer sequences which were derived from the CVH sequence (accession number AB004836), Dazl sequence (accession number AY211387), or β-actin sequence (accession number NM 205518) were:

V-1
(SEQ ID NO. 1)
GCTCGATATGGGTTTTGGAT
V-2
(SEQ ID NO. 2)
TTCTCTTGGGTTCCATTCTGC
Dazl-1
(SEQ ID NO. 3)
GCTTGCATGCTTTTCCTGCT
Dazl-2
(SEQ ID NO. 4)
TGC GTC ACA AAG TTA GGC A
Act-RT-1
(SEQ ID NO. 5)
AAC ACC CCA GCC ATG TAT GTA
Act-RT-2
(SEQ ID NO. 6)
TTT CAT TGT GCT AGG TGC CA

Primers V-1 and V-2 were used to amplify a 751 bp fragment from the CVH transcript. Primers Dazl-1 and Dazl-2 were used to amplify a 536 bp fragment from the Dazl transcript. Primers Act-RT-1 and Act-RT-R were used to amplify a 597 bp fragment from the endogenous chicken β-actin transcript. PCR reactions were performed with AmpliTaq Gold (Applied Biosystems) following the manufacturer's instructions (FIG. 2).

### EXAMPLE 3

### PGCs Express the CVH Protein

Protein was extracted from freshly isolated PGCs using the T-Per tissue protein extraction kit (Pierce). Protein from cells was extracted by lysing the cells in 1% NP₄0; 0.4% deoxycholated 66 mM EDTA; 10 mM,Tris, pH7.4. Samples were run on 4-15% Tris-HCL ready gel (Bio-Rad). After transfer onto a membrane, Western blots were performed with Super Signal West Pico Chemiluminescent Substrate kits (Pierce) as instructed. A rabbit anti-CVH antibody was used as a primary antibody (1:300 dilution) and a HRP-conjugated goat anti-rabbit IgG antibody (Pierce, 1:100,000) was used as a secondary antibody (FIG. 3).

### EXAMPLE 4

### Cultured PGCs Express Telomerase

Primordial germ cells were pelleted and washed with PBS before being frozen at −80° C. until analysis. Cell extracts were prepared and analyzed according to the manufacturer's directions using the TRAPeze Telomerase Detection Kit (Serologicals Corporation) which is based upon the Telomeric Repeat Amplification Protocol (TRAP) (Kim, N. et al. Specific association of human telomerase activity with immortal cells and cancer. Science 266, 2011-2014, 1994). FIG. 4.

### EXAMPLE 5

### Embryonic Germ (EG) Cells can be Derived from Cultures of PGCs

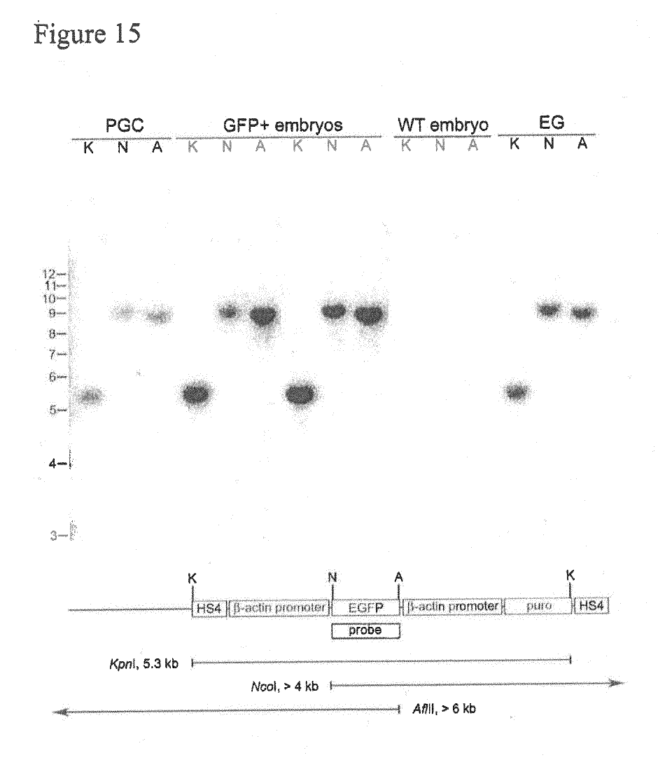

Chicken EG cells have been derived from PGCs by allowing the cells to attach to the plate, removing FGF, SCF and chicken serum, and culturing the cells under the same conditions used for ES cell culture (van de Lavoir et al, 2006 High Grade Somatic Chimeras from Chicken Embryonic Stem Cells, Mechanisms of Development 12, 31-41; van de Lavoir and Mather-Love (2006) Chicken Embryonic Stem Cells; Culture and Chimera Production, Methods in Enzymology, in press). The morphology of the cEG cells is very similar to that of the cES cells (FIGS. 5A and 5B). When cEG cells are injected into Stage X (E-G&K) embryos, they have the ability to colonize somatic tissues and make chimeras that, as juveniles, appear identical to chimeras made with cES cells Chicken EG cells are observed in both newly derived and clonally derived transgenic PGC lines. Southern analysis of EG cells derived from GFP positive PGCs demonstrate that EG cells originate from the PGCs (FIG. 15).

### EXAMPLE 6

### Cultured Male PGCs Give Rise to Functional Gametes in Roosters

Male primordial germ cell lines were derived from individual Barred Rock embryos. After establishment of the line, the cells were injected into Stage 13-15 (H&H) embryos. Phenotypically, the hatched chicks resembled White Leghorns. The males were reared to sexual maturity and have been mated to Barred Rock hens (Table 1). Black offspring were indicative of germline transmission of the injected PGCs. The rate of germline transmission of the roosters varied from <1% to 86% (Table 1).

TABLE 1
Germline transmission of male primordial germ cells injected into
the vasculature of Stage 14-15 (H&H) embryos.

| | | | # cells | # | % germline |
|---|---|---|---|---|---|
| Cell line | Sex | Age | injected | Roosters | transmission* |
| PGC13 | M | 40 | 1200 | 3 | 0.1, 1.5, 17 |
| | | 110 | 2500-3000 | 5 | 1,1, 1.5, 3, 84 |
| PGC21 | M | 44 | 1500 | 3 | 10, 16, 21 |
| PGC34 | M | 47 | 3000 | 3 | 42, 74, 80 |
| PGC35 | M | 35 | 3000 | 7 | 15, 23, 47, 61, 80, 85, |
| | | | | | [symbol] |
| PGC51 | M | 47 | 3000 | 1 | 11 |
| PGC54 | M | 47 | 3000 | 4 | 0.5, 2, 20, 24 |
| PGC80 | M | 29 | 3000 | 1 | 55 |
| PGC84 | M | 50 | 3000 | 1 | 70 |

*Each value represents the rate of germline transmission of one chimera [symbol]
indicates data missing or illegible when filed

PGCs may also be injected into the subgerminal cavity of stage X embryos. 1000 or 5000 PGCs were injected after 209 days of culture into irradiated embryos. Hatched male chicks were grown to sexual maturity and bred to test for germline transmission. In 3 out of 4 roosters tested germline transmission observed in varying frequency of 0.15 to 0.45%. This indicates that PGCs can colonize the germline when injected before gastrulation. Germline transmission of male PGCs has not been observed in 1,625 offspring of 14 female chimeras.

### EXAMPLE 7

### Cultured Female PGCs Give Rise to Functional Gametes in Hens

Female PGCs from Barred Rock embryos that were cultured 66 days were injected into Stage 13-16 (H&H) White Leghorn embryos and all hatched chicks were phenotypically White Leghorns. The hens were reared to sexual maturity and have been mated to Barred Rock roosters. Female PGCs transmitted through female chimeras at frequencies up to 69%. (Table 2).

TABLE 2
Germline transmission of female primordial germ cells injected into the
vasculature of Stage 14-15 (H&H) embryos.

| Cell | | | # cells | # hens | % germline |
|---|---|---|---|---|---|
| line | Sex | Age | injected | tested | transmission* |
| PGC56 | F | 66 | 3000 | 5 | 1, 2, 6, 52, 69 |
| PGC85 | F | 47 | 3000 | 10 | 0, 0, 0, 2, 2, 4, 5, 10, 11, 12 |

*Each value represents the rate of germline transmission of one chimera.

Female PGCs were also injected into male recipient White Leghorn embryos. The male chimeras were reared to sexual maturity and bred to Barred Rock hens. Germline transmission of female PGCs was not observed in 506 offspring of three roosters tested.

### EXAMPLE 8

### Offspring Derived from PGCs are Reproductively Normal

Three male and 4 female non-transgenic PGC derived offspring were bred together. Between 53 and 100% of the eggs were fertile (Table 3) and between 79 and 100% of the fertile eggs resulted in a hatched embryo (Table 3), indicating that PGC derived offspring are reproductively normal.

TABLE 3
Reproductive parameters of PGC offspring obtained from germline chimeric
roosters.

| | | | Infertile/ | | | |
|---|---|---|---|---|---|---|
| | | Eggs | early | | # | % of fertile |
| Rooster | Hen | set | dead | Fertility % | Hatched | embryos |
| IV9-1-7&IV9-1-8 | IV9-1-1 | 36 | 17 | 53 | 15/19 | 79 |
| IV9-1-2 & IV9-1-8 | IV9-1-4 | 33 | 5 | 85 | 27/28 | 96 |
| IV9-1-7&IV9-1-8 | IV9-1-5 | 38 | 8 | 79 | 28/30 | 93 |
| IV9-1-2 | IV9-1-6 | 12 | 0 | 100 | 12/12 | 100 |

### EXAMPLE 9

Primordial germ cells have been isolated from Stage 14-17 embryos and shown to contribute to the germline (see Examples 1-8). At this time, PGCs are circulating in the vascular system. Prior to formation of the vascular system, the PGCs were situated in the germinal crescent, which lies anterior to the embryo proper. The precursors of PGCs in the germinal crescent are not well understood but it is generally presumed that PGCs are derived from cells in the area pellucida of the Stage X (Eyal-Giladi and Kochav) embryo (Petitte, J. N. 2002. The Avian germline and Strategies for the Production of Transgenic Chickens. Journal of Poultry Science 39, 205-228). During their residence in the Stage X embryo, PGCs cannot be identified using the classical morphological criteria that are used for their identification in the germinal crescent. Surprisingly, placement of dispersed cells from Stage X Barred Rock embryos was shown to give rise to PGCs and contribute to the germline. We demonstrated this principle by collecting blastoderms individually and mechanically dispersing them by trituration in a Pasteur pipette. The cells were washed and plated into a 48 well plate previously seeded with irradiated BRL cells containing the medium described in Example 1. The cultures were passaged for the first time 6-10 days after seeding. Thereafter the passaging depended on the concentration of PGCs present. Two male cell lines (PGC-A12 and PGC-B11) were established and injected into recipient embryos as described in Example 6 after 45 and 36 days in culture respectively. Five male chimeras were produced from each cell line. As shown in Table 4, the Barred Rock phenotype was transmitted through the germline in 3 of the 10 males demonstrating that cells destined to become functional PGCs could be cultured in the medium that was provided.

TABLE 4
Germline transmission of PGCs derived from Stage X (EG&K) embryos

| | | | Total | | |
|---|---|---|---|---|---|
| | | | number | # black | % germline |
| ID Bird | Cell line | sex | offspring | offspring | transmission |
| IV-045-003 | PGC-A12 | M | 95 | 0 | 0 |
| IV-045-004 | PGC-A12 | M | 107 | 1 | 1 |
| IV-045-017 | PGC-A12 | M | 74 | 0 | 0 |
| IV-045-025 | PGC-A 12 | M | 61 | 29 | 48 |
| IV-045-031 | PGC-A12 | M | 23 | 0 | 0 |
| IV-045-012 | PGC-B11 | M | 59 | 0 | 0 |
| IV-045-035 | PGC-B11 | M | 56 | 0 | 0 |
| IV-045-040 | PGC-B11 | M | 41 | 1 | 1 |
| IV-045-053 | PGC-B11 | M | 90 | 0 | 0 |
| IV-045-059 | PGC-B11 | M | 93 | 0 | 0 |

### EXAMPLE 10

### Sensitivity of PGCs to Neomycin and Puromycin

The sensitivity of PGCs to puromycin and neomycin was determined to establish the concentration of puromycin and neomycin required to allow the growth of cells that express antibiotic resistance under the control of the CX-promoter which is strongly expressed in all tissues). These experiments demonstrated that a concentration of 300 μg/ml neomycin for 10 days is necessary to eliminate all non-transfected cells. A concentration of 0.5 μg/ml puromycin was sufficient to eliminate PGCs within 7-10 days.

### EXAMPLE 11

### Genetic Modification of PGCs

Twenty microgram (20 μl) of a Notl linearized cx-neo transgene (see FIG. 6) was added to a population of 5.8×10⁶ PGCs that had been in culture for 167 days. The cells and DNA were resuspended in 800 ul of electroporation buffer and 8 square wave pulses of 672 volts and 100 μsec duration were applied. After ten minutes, the cells were resuspended in culture medium and aliquoted into 24-well plates. Two days after electroporation, 300 μg of neomycin were added per ml of medium to select cells that were expressing the cx-neo transgene. The cells were kept under selection for 19 days. After 19 days, the cells were taken off selection and expanded for analysis. A proportion of the PGCs was kept under 300 μg/ml for another 31 days demonstrating that the PGCs were functionally resistant to the antibiotic.

Referring to FIG. 6, for the plasmid control, the cx-neo plasmid DNA was linearized with Notl and then digested with EcoRI or BamHI to produce a fragment that is slightly smaller (5 kb) than the intact plasmid which is shown by the Hindlll digestion. Internal fragments of approximately 2 kb of the cx-neo plasmid were released by digestion with Styl or Ncol. A larger internal fragment of approximately 2.6 kb was released by digestion with EcoRI and Kpnl. Digestion of genomic DNA from the line of PGCs with EcoRI, BamHI and Hindlll revealed bands that are larger than 6 kb illustrating that the cx-neo transgene was incorporated into the PGC genome. The internal fragments revealed in plasmid DNA following digestion with Styl, Ncol and EcoRI with Kpnl were also present in genomic DNA from the PGCs indicating that the cx-neo transgene was integrated into the PGC genome without alteration. Using conventional transgene construction techniques, additional elements can be incorporated such as regulatory elements, tissue specific promoters and exogenous DNA encoding proteins are examples.

As noted above, the performance of genetic modifications in PGCs to produce transgenic animals has been demonstrated in only a very few species. Analogous genetic manipulations can be achieved in chicken PGCs by referring to those achieved using ES cells in mice. In mice, the separate use of homologous recombination followed by chromosome transfer to embryonic stem (mES) cells for the production of chimeric and transgenic offspring is well known. Powerful techniques of site-specific homologous recombination or gene targeting have been developed (see Thomas, K. R. and M. R. Capecchi, Cell 51: 503-512, 1987; review by Waldman, A. S., Crit. Rev. Oncol. Hematol. 12: 49-64, 1992). Insertion of cloned DNA (Jakobovits, A, Curr. Biol. 4: 761-763, 1994) and manipulation and selection of chromosome fragments by the Cre-loxP system techniques (see Smith, A. J. et al, Nat. Genet. 9:376-385, 1995; Ramirez-Solis, R. et al. Nature 378:720-724, 1995; U.S. Pat. Nos. 4,959,317; 6,130,364; 6,130,364; 6,091,001; 5,985,614) are available for the manipulation and transfer of genes into mES cells to produce stable genetic chimeras.

The genome of primordial germ cells is generally believed to be in a quiescent state and therefore the chromatin may be in a highly condensed state. Extensive testing of conventional electroporation protocols suggest that special methods are needed to introduce genetic modifications into the genome of PGCs. As described below, the transgenes may be surrounded with insulator elements derived from the chicken p-globin locus to enhance expression. The inclusion of the p-globin insulator elements routinely produces clones that can be grown, analyzed, and injected into recipient embryos.

The conventional promoters that are used to drive expression of antibiotic (e.g. neomycin, puromycin, hygromycin, his-D, blasticidin, zeocin, and gpt) resistance genes are expressed ubiquitously. Typically, the promoters are derived from “housekeeping” genes such as β-actin, CMV, or ubiquitin. While constitutive promoters are useful because they are typically expressed at high levels in all cells, they continue to be expressed in most if not all tissues throughout the life of the chicken. In general, expression should be limited to only the tissue and stage of development during which expression is required. For selection of primordial germ cells, the period during which expression is required is their residence in vitro when the antibiotic is present in the media. Once the cells have been inserted into the embryo, it is preferable to terminate expression of the selectable marker (i.e. the antibiotic resistance gene). To restrict expression of the antibiotic resistance genes, the “early response to neural induction” (ERNI) promoter is used. An ERNI is a gene that is selectively expressed during the early stages of development (e.g. Stage X (E-G&K)) and in culture, and therefore, this promoter is used to drive expression of antibiotic resistance genes to select PGCs carrying a genetic modification. Since ERNI is only expressed during the early stages of development, the genes that confer antibiotic resistance are not expressed in the mature animals.

### EXAMPLE 12

### Homogeneity of Long Term PGC Cell Cultures

To determine the homogeneity of PGC cultures after long-term culture, ES, EG, DT40 (chicken B cell line) and PGCs were stained with anti-CVH, an antibody against the chicken vasa homologue and the 1B3 antibody (Halfter, W, Schurer, B, Hasselhorn, H. M, Christ, B, Gimpel, E, and Epperlein, H. H, An ovomucin-like protein on the surface of migrating primordial germ cells of the chick and rat. Development 122, 915-23. 1996)). Expression of the CVH antibody is restricted to germ cells and therefore, the anti-CVH antibody is a reliable marker for them. The 1B3 antigen recognizes an ovomucin-like protein present on the surface of chicken PGCs during their migration and colonization of the gonad.

Cells were washed in CMF/2% FBS, fixed in 4% paraformaldehyde for 5 minutes and washed again. The cell aliquots to be stained for vasa were permeabilized with 0.1% TritonX-100 for 1-2 minutes. Primary antibody was added for 20 minutes, cells were washed twice and incubated with a secondary antibody (Alexa 488 anti-rabbit IgG for CVH and control and Alexa 488 anti-rabbit IgM for 1B3) for 15 minutes. As controls, aliquots of cells were stained only with second antibody. After an additional 2 washes the cells were prepared for FACS analysis.

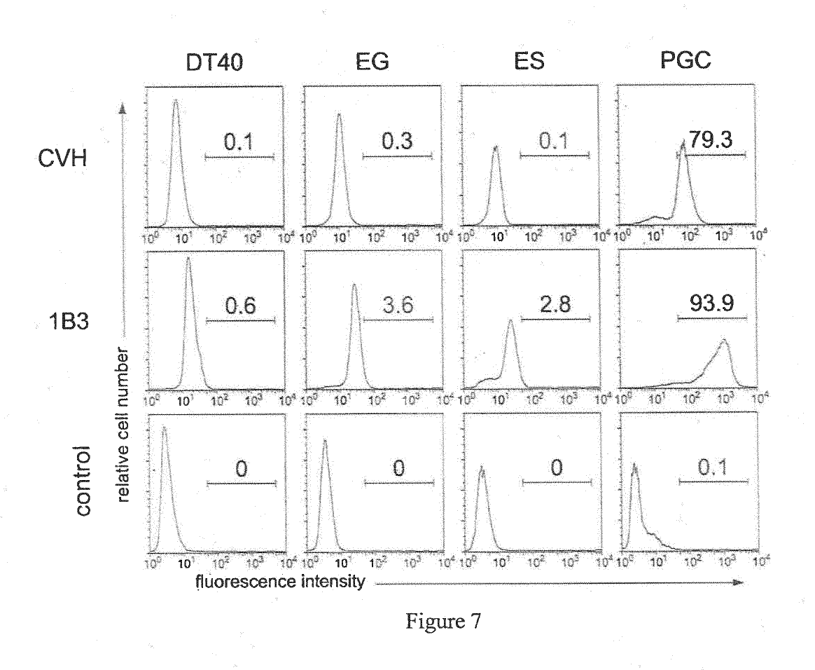

Referring to FIG. 7, DT40, ES and EG cells all show background when stained with CVH and the 1B3 Ab. PGCs, however, stain much stronger with both the CVH and the 1B3 antibody. There is a small population of PGCs, which do not stain for either CVH or 1B3 indicating that a small proportion of the cells do not display the PGC phenotype. Two parental PGC lines and 4 transfected cell lines (G-09, P84, P97/6 and P97/33) derived from the PGC 13 parental cell line, were tested with the vasa and 1B3 antibody (PGC 13 and 102). All show the same pattern indicating that the various PGC cultures contain the same high proportion of cells expressing the PGC phenotype.

### EXAMPLE 13

### Genetic Modification of Primordial Germ Cells

Electroporation with a circular CX-GFP plasmid revealed that the rate of transient transfection in PGCs varied between 1-30%. Using 8 Square wave pulses of 100 ( μsec and 800V, we obtained a PGC cell line carrying a CX-neo construct, that was designated G-09. See FIG. 6. The integration of the construct was evaluated using Southern blot analysis. The isolation of this stably transfected line, however, was a non-recurring event. With the exception of G-09, stable transfection of PGCs was not achieved after electroporating 17×10⁷ PGCs with linearized constructs in 37 transfection experiments using both square wave and exponential decay pulses. In each of these experiments, the number of PGCs varied from 1×10⁶ to 10×10⁶. The following promoters, used widely in ES cell research in mouse, chicken and human were tested: the CX promoter, also called CAG (Niwa, H, Yamamura, K, and Miyazaki, J, Efficient selection for high-expression transfectants with a novel eukaryotic vector. Gene 108, 193-9.1991)), which contains the chicken p-actin promoter with a CMV enhancer, the PGK promoter, the MC1 promoter and the Ubc promoter. None of these promoters increased transfection efficiency. To allow expression of selectable markers and clonal derivation of genetically modified cell lines, insulators have been used with integrated constructs.

Insulators are DNA sequences that separate active from inactive chromatin domains and insulate genes from the activating effects of nearby enhancers, or the silencing effects of nearby condensed chromatin. In chickens, the 5′HS4 insulator located 5′ of the β-globin locus has been well characterized by Felsenfeld and colleagues (Burgess-Beusse, B, Farrell, C, Gaszner, M, Litt, M, Mutskov, V, Recillas-Targa, F, Simpson, M, West, A, and Felsenfeld, G. (2002)). The insulation of genes from external enhancers and silencing chromatin. Proc. Natl. Acad. Sci. USA99 Suppl. 4, 16433-7. This insulator protects the P-globin locus from an upstream region of constitutively condensed chromatin. We assembled a transgene with the chicken β-actin promoter driving neomycin resistance using the chicken 3-globin 5′HS4 sequence as insulators both 5′ and 3′ of the chicken β-actin-neo cassette.

The 250 bp core sequence of hypersensitive site 4 from the chicken β-globin locus was PCR amplified with the following primer set:

HS4-Bam-F:
(SEQ ID. NO. 7)
AGGATCCGAAGCAGGCTTTCCTGGAAGG
HS4-Bgl-R:
(SEQ ID. NO. 8)
AAGATCTTCAGCCTAAAGCTTTTTCCCCGT

The PCR product was cloned into pGEM-T and sequenced. A tandem duplication of the HS4 site was made by digesting the HS4 in the pGEM clone with BamHI and Bglll to release the insert, and Bglll to linearize the vector. The HS4 fragment was ligated to the vector containing a copy of the HS4 insulator. Clones were screened and one was selected in which the two copies of HS4 are in the same orientation. This is called 2X HS4.

### EXAMPLE 14

### Bulk Selection Using HS4-β-Actin-Neo

β-actin neo was obtained from Buerstedde (clone 574) and transferred into pBluescript. 2X HS4 was then cloned at both the 5′ and 3′ ends of β-actin neo to produce HS4-β-actin neo. Eight transfections were performed using this construct. For each transfection 5×10⁶ PGCs were resuspended in 400 μl electroporation buffer (Specialty Media) and 20 ug of linearized DNA was added. One Exponential Decay (ED) pulse (200V, with 900-1100 μF) or eight Square Wave (SW) pulses (250-350V, 100( μsec) were given. After transfection, the cells were grown for several days before neomycin selection (300 μg/ml) was added. Each transfection was grown as a pool. Resistant cells were isolated from 5 of 8 transfections

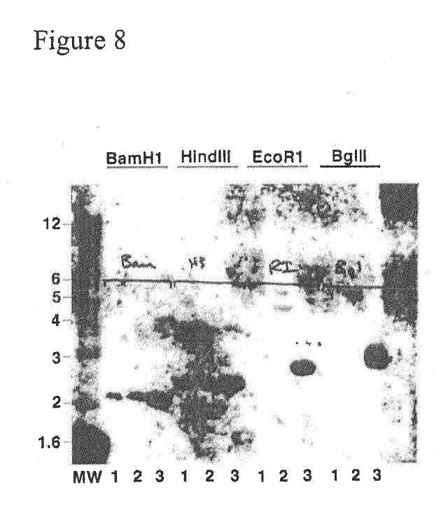

Southern analysis was performed on 2 pools of transfected cells (FIG. 8). Two μg genomic DNA from PGC lines P84 and P85 and 20 pg of plasmid (HS4-β-actin neo) were digested. Digests were run on a 0.7% gel, transferred by capillary transfer in 10×SSC to nylon membrane overnight, and probed with radiolabeled neo gene sequences for 2 hours in Rapid Hyb (Amersham). After washing, the blot was exposed to film overnight at −80° C. Referring to FIG. 8, Lanel is P84, Lane 2 is P85 and Lane 3 is the plasmid. For the plasmid control the HS4-β-actin-neo plasmid DNA was linearized with Notl. To obtain a 2.3 Kb internal fragment the PGC DNA and the linearized plasmid were digested with BamHI. Both P84 and P85 show an internal fragment of 2.3 Kb in size. A larger internal fragment of approximately 2.6 Kb was released by digestion with Hindlll. Again this internal fragment is present in both the P84 and P85 digests. Digestion of genomic DNA of P84 and P85 with EcoRI and Bglll should reveal bands larger than 2.9Kb if the transgenes are integrated into the genome. In P84 no junction fragments are seen, indicating that P84 is a composite of several different clones. In P85, junction fragments of 4.5-5kb are present in the EcoRI digestion and a junction fragment of 5 Kb is present in the Bglll digestion indicating that P85 is integrated into the genome and that the culture is comprised substantially from one clone. This example shows the utility of insulators as a preferred element of a construct for reliable expression of selectable markers in primordial germ cells.

### EXAMPLE 15

### Clonal Derivation of Genetically Modified PGCs

The following examples show that genetically modified lines of primordial germ cells can be clonally derived.

First, β-actin-eGFP was made. The eGFP gene was released from CX-eGFP-CX-puro with Xmnl and Kpnl, β-actin was released from HS4-β-actin puro with EcoRI and Xmnl, and the two were cloned as a 3-way ligation into pBluescript digested with EcoRI and Kpnl to produce p-actin EGFP. Then, β-actin eGFP was released with BamHI and Kpnl (blunted with T4 DNA polymerase) and cloned into HS4-β-actin puro digested with Bglll and EcoRV.

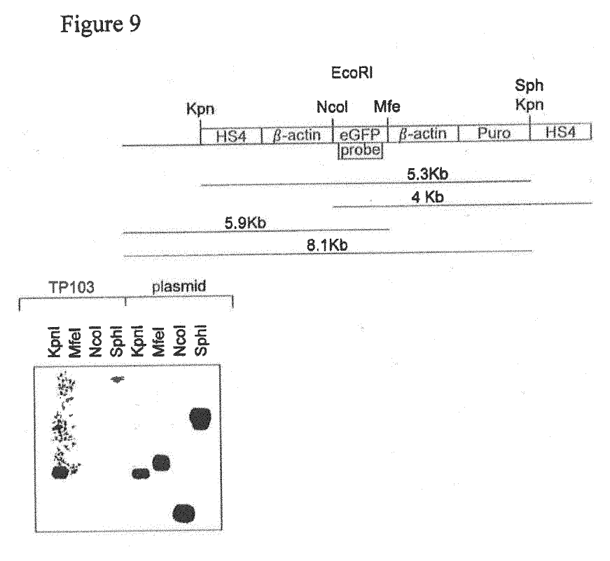

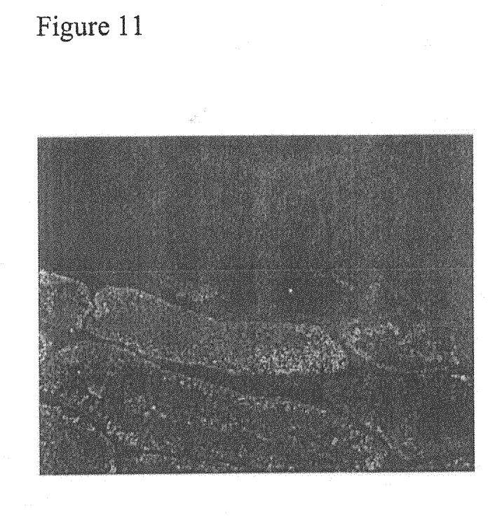

Five transfections were performed using this construct. For each transfection 5×10⁶ PGCs were resuspended in 400 uf electroporation buffer (Specialty Media) and 20 ug of linearized DNA was added. An ED pulse (150-200V; 900 μF) or SW (350V, 8 pulses, 100 μsec) pulses were given. After transfection the cells were plated into individual 48 wells and grown for several days before selection (0.5 μg/ml) was added. A total of 5 clones were observed in 4 of the 5 transfections. One clone TP103 was analyzed by Southern (FIG. 9). Referring to FIG. 11, the plasmid control DNA was linearized with Notl. An internal fragment was released by digesting the DNA with Kpnl. In both TP103 and the plasmid, a fragment of the same size was released. Digestion of genomic DNA of TP103 with Ncol, Mfel, and Sphl should reveal bands that are larger than the corresponding lanes of digested plasmid DNA. No band is seen in the lane of Mfel digested TP103 genomic DNA, which may be due to the band being too large. In the lanes representing the Ncol and Sphl digestions, fragments have been released in the TP 103 genomic DNA that are substantially larger than the fragments released in the plasmid DNA, indicating that the transgene is incorporated into the genome of the TP103 cell line.

### Clonal Derivation of HS4-β-Actin-Puro.

First, p-actin puro was made by a 3-way ligation of puro from CX-EGFP-CX-puro (Xmnl-EcoRI), β-actin from β-actin neo in pBS (see above)(Sal-Xmnl), and pBluescript (Sall-EcoRI). Next, β-actin puro was cloned into pBS containing two copies of 2X HS4 by ligating BamHI digested β-actin puro into BamHI/SAP treated 2X HS4 vector.

Three transfections were performed using this construct. For each transfection 4-5×10⁶ PGCs were resuspended in 400 ul electroporation buffer (Specialty Media) and 20 μg of linearized DNA was added. An ED pulse was given of 200V, 9000 μF. After transfection the cells were plated into individual 48 wells and grown for several days before selection (0.5 μg/ml) was added. No colonies were seen in 2 transfections. Two colonies were isolated from the third transfection.

### Clonal Derivation of HS4-cx-eGFP-cx-Puro.

Three transfections were performed with HS4-cx-eGFP-cx-Puro. 5×10⁶ PGCs were resuspended in 4 μl electroporation buffer (Specialty Media) and 20 μg of linearized DNA was added. Eight SW pulses of 350V for 100 μsec was given to each transfection. After transfection the cells were plated in individual 48 wells, grown for several days before puromycin selection (0.5 μg/ml) was added. A total of 16 clones were isolated from 2 transfections.

Clonal Derivation of cx-Neo.

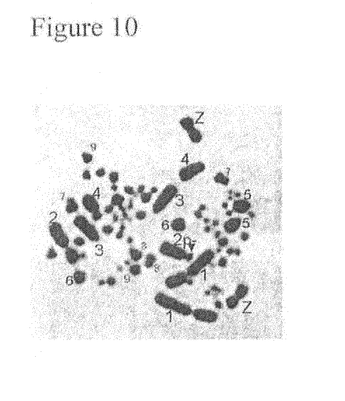

The PGC 13 cell line was electroporated with a plasmid carrying a cx-neo selectable marker. After exposure to neomycin a cell line was derived that was resistant to neomycin (G-09). The karyotype of this cell line was determined and all cells exhibited a deletion in the p-arm of chromosome 2 (Table 5 and FIG. 10). These data demonstrate that G-09 was clonally derived from a PGC carrying a signature deletion in the p-arm of chromosome 2.

TABLE 5
Chromosomal analysis of G-09 cell line.

| | Chromosomes | | | | | | |
|---|---|---|---|---|---|---|---|
| Cell | 1 | 2 | 2p- | 3 | 4 | Z | Micros |
| 1 | 2 | 1 | 1 | 2 | 2 | 2 | 69 |
| 2 | 2 | 1 | 1 | 2 | 2 | 2 | 44 |
| 3 | 2 | 1 | 1 | 2 | 2 | 2 | 56 |
| 4 | 2 | 1 | 1 | 2 | 2 | 2 | 56 |
| 5 | 2 | 1 | 1 | 2 | 2 | 2 | 65 |
| 6 | 2 | 1 | 1 | 2 | 2 | 2 | 67 |
| 7 | 2 | 1 | 1 | 2 | 2 | 2 | 59 |
| 8 | 1 | 0 | 1 | 1 | 2 | 1 | 38 |
| 9 | 2 | 1 | 1 | 2 | 2 | 2 | 65 |
| 10 | 2 | 0 | 1 | 2 | 2 | 2 | 55 |
| 11 | 2 | 1 | 1 | 2 | 2 | 2 | 43 |
| 12 | 2 | 1 | 1 | 2 | 2 | 2 | 59 |
| 13 | 2 | 0 | 1 | 2 | 2 | 2 | 55 |
| 14 | 2 | 0 | 1 | 2 | 2 | 2 | 33 |
| 15 | 1 | 1 | 1 | 2 | 2 | 2 | 56 |
| 16 | 2 | 1 | 1 | 2 | 2 | 2 | 62 |

### EXAMPLE 16

### Tissue Specific Expression of Selectable Markers in PGCs

The gene ERNI is expressed from the pre-primitive streak stage in the chicken embryo and is an early response gene to signals from Hensen's node Streit, A, Berliner, A. J, Papanayotou, C, Sirulnik, A, and Stern, C. D. (2000). Initiation of neural induction by FGF signalling before gastrulation. Nature 406, 74-8. Furthermore ERNI is expressed in chicken ES cells Acloque, H, Risson, V, Birot, A, Kunita, R, Pain, B, and Samarut, J. (2001). Identification of a new gene family specifically expressed in chicken embryonic stem cells and early embryo. Mech Dev 103, 79-91. The ERNI gene (also called cENS-1) has an unusual structure in which a single long open reading frame is flanked by a 486 bp direct repeat, in addition to unique 5′ and 3′ UTR sequences. Based on the idea that this structure is reminiscent of a retroviral LTR-like structure, Acloque et al. 2001 assayed different portions of the cDNA sequence for promoter/enhancer activity and found that a region of the unique sequence in the 3′ UTR acts as a promoter. PCR primers were designed essentially as described (Acloque et al, 2001) to amplify an 822 bp fragment of the 3′ UTR of the ERNI gene. After amplification of the ERNI sequences, they were cloned upstream of the neomycin-resistance gene, with an SV40 polyA site, to generate ERNI-neo (1.8 kb). The 2X HS4 insulator was then cloned on either side of the ERNI-neo selectable marker cassette.

Two transfections were performed with HS4-Erni-neo. 5×10⁶ PGCs were resuspended in 400 μl electroporation buffer (Specialty Media) and 20 μg of linearized DNA was added. In the first transfection a single ED pulse of 175V, 9000 μF was given and in the second transfecton, 8 SW pulses of 100 (isec and 350V were given. After transfection the cells were plated in individual 48 wells, grown for several days before neomycin selection (300 μg/ml) was added. In the first transfection (ED pulse) 5 colonies were isolated, and in the second transfection (SW pulses) 11 colonies were isolated.

The isolation of stably transfected clones indicates that ERNI is expressed in PGCs and can be used as a tissue specific promoter.

### EXAMPLE 17

### Contribution of Transfected PGCs to the Germline

PGCs were transfected with HS4-Bactin-GFP and injected into the vasculature of Stage 13-15 (H&H) embryos. At D18, gonads were retrieved, fixed, sectioned and stained with the CVH antibody to identify the germ cells. The stained sections were then analyzed for the presence of GFP positive cells in the gonads. GFP positive germ cells were found in both male (FIG. 11) and female gonads. Examination of histological preparations of brain, heart muscle and liver of these embryos showed only four green cells in one slide. These data demonstrate that a few cultured PGCs are found in ectopic locations but that the vast majority of cultured PGCs preferentially colonize the germline.

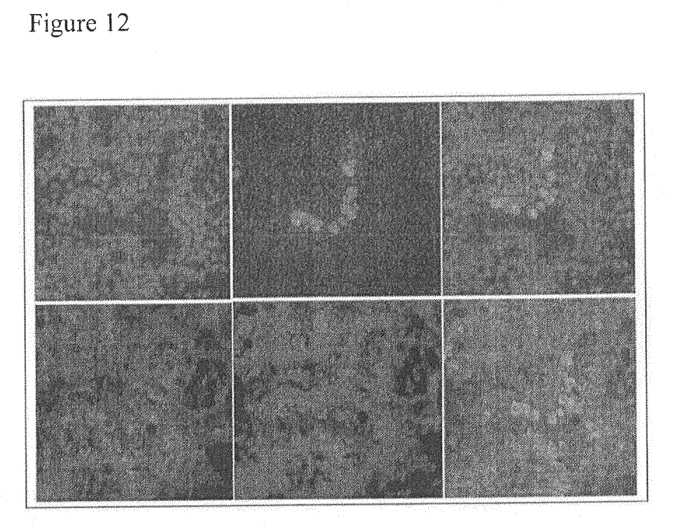

To determine that the GFP positive cells were germ cells the sections were stained with the anti-CVH antibody. As can be seen in FIG. 12, the GFP positive cells also stain for the CVH protein, indicating that the GFP positive cells are germ cells.

Referring to FIG. 12, GFP positive cells are present in this section and the DAPI/GFP panel shows that these GFP positive cells are located within the seminiferous tubule. When germ cells are stained with the anti-CVH antibody they exhibit a intense red stained ring that delineates the cytoplasm of the germ cells. The DAPI/CVH panel shows that these cells are located within the seminiferous tubule. The last panel shows that the GFP positive cells also stain for CVH and that the seminiferous tubules contains CVH positive germ cells that are GFP negative.

### EXAMPLE 18

### Germline Transmission of Genetically Modified PGCs

Barred Rock PGCs transfected with one of the following transgenes: βactin-neo, Bactin-eGFP-Bactin-puro, or cx-eGFP-cx-puro were injected into the vasculature of Stage 13-14 (H&H) embryos. The chicks were hatched, the roosters were grown to sexual maturity and bred to Barred Rock hens to determine germline transmission of the transgene. All black offspring were PGC derived and were tested for the presence of the transgene (Table 6). The rate of germline transmission was calculated by dividing the number of black chicks by the total number of chicks that were scored for feather color (Table 6).

TABLE 6
Germline transmission of genetically modified primordial germ cells.

| | Parental | Age of | | Roosters | # | |
|---|---|---|---|---|---|---|
| Cell line | cell line | cells | Construct | tested | offspring | % germline |
| TP84 | PGC13 | 267 | βactin-neo | 5 | 892 | 0, 0, 0, 0, 0 |
| TP85 | PGC13 | 260-267 | βactin-neo | 12 | 2462 | 0, 0, 0, 0, 0, 0, 0, |
| | | | | | | 0, 0, 0, 0.5, 1 |
| TP103/38 | PGC54 | 134-138 | βactin- | 8 | 758 | 0, 1, 11, 12, 13, 16, 28, 92 |
| | | | GFP | | | |
| TP112/44 | PGC13 | 280 | cx-GFP | 4 | 168 | 0, 0, 0, 4 |
| TP112/21 | PGC13 | 280 | cx-GFP | 3 | 378 | 0, 1, 10 |

### EXAMPLE 19

### Transgenes are Inherited in a Mendelian Fashion

Black offspring from matings between chimeric roosters carrying Barred Rock PGCs that were genetically modified to include one of Pactin-neo, Pactin-GFP, or cx-GFP were analyzed for the presence of the transgene. As shown in Table 7 the transgene is inherited by approximately 50% of the PGC offspring, indicating Mendelian inheritance.

TABLE 7
Mendelian segregation of the transgene.

| | | # non-transgenic | # transgenic |
|---|---|---|---|
| Construct | # black offspring | offspring | offspring |
| βactin-neo | 3 | 1 | 2* |
| βactin-GFP | 176 | 93 | 83* |
| cx-GFP | 23 | 9 | 14* |

*Not significantly different from the expected 1:1 ratio of transgenic:non-transgenic offspring by Chi-square analysis

### EXAMPLE 20

### Ubiquitous Expression of Transgenes in Offspring of Chimeras Carrying Genetically Modified PGCs

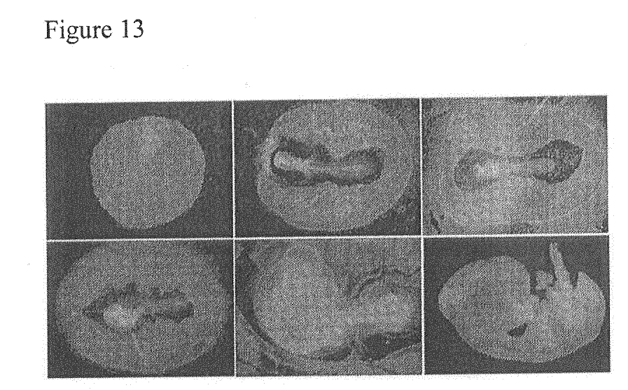

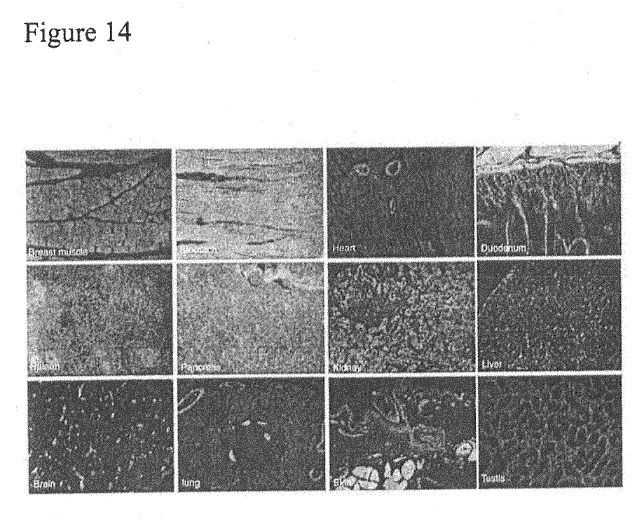

Chimeras carrying PGCs in which Bactin-GFP was stably integrated into the genome were mated with wild type hens and the embryos were scored for expression of GFP. Examples of expression in embryos are shown in FIG. 13 which shows that GFP is expressed in all tissues of the transgenic offspring up to Stage 34 (H&H) of development. In older animals, tissues were prepared for histological examination using frozen sections. Tissues from pancreas, skin, lung, brain, ovary, kidney, bursa, duodenum, breast, heart, liver, and spleen of 1 to 2 week-old chicks demonstrated that expression remained ubiquitous in animals post hatching (FIG. 14).

### EXAMPLE 21

### HS4 Containing Transgenes are Inserted into Promoter Regions in the Chicken Genome

To address whether the HS4-containing constructs were preferentially inserted into particular regions of the genome that would avoid silencing and permit expression of the selectable markers, we identified the transgene insertion sites in our clonal transfected PGC lines. Genomic DNA was extracted from transfected PGC lines and digested with a restriction enzyme that either did not cut the transgene, or one that cut once in the HS4 element. The DNA was self-ligated and transformed into E. coli. The cells were plated on ampicillin plates to isolate colonies containing the amp gene from the plasmid joined to genomic sequences flanking the vector.

The plasmids were purified and sequenced from 31 of the HS4-construct transfected PGC lines. We performed BLAT (UCSC Chicken Genome Browser Gateway) and BLAST (NCBI) searches to map the genomic locations of each of the insertions. Strikingly, 25 of the 31 HS4-containing constructs were inserted into CpG islands, which are commonly found near promoter regions, especially those of housekeeping genes. Of the insertions in CpG islands, genes could be found associated with most of them (23/25), either a known gene or a novel gene as defined by ESTs (Table 8). CpG islands often extend from several hundred base pairs upstream of the transcription start site, through the first exon, and into the first intron, and insertions were found in all of these regions. There was no bias in the transcriptional orientation of the vector relative to the endogenous gene. Many of these genes are predicted to be expressed in PGCs, based on their known functions as housekeeping genes, such as isocitrate dehydrogenase, aldehyde dehyodrogenase, and a mitochondrial solute carrier. Seven of the insertions were in novel genes, as defined by ESTs. Five of these ESTs were originally cloned from gonad or PGC libraries, suggesting that these genes may also be expressed in our PGC cell lines. Three of the insertions in genes were not in CpG islands but rather in more distal introns. Five of the insertions were in regions without any obvious genes. Three of these insertions were very near LINE or satellite repeats.

TABLE 8
Insertion of transgenes containing HS4 sequences into PGCs.

| | | Chromosomal | | | CpG | |
|---|---|---|---|---|---|---|
| Clone | Construct | location | Gene | Position | island | Accession number |
| TP85 | 11S4 BN | Chr19: 6,414,674 | Aldehyde | Promoter | Yes | NM 001006223.1 |
| | | | dehydrogenase 3 | | | |
| | | | family member A2 | | | |
| TP97/6 | HS4 BP | ND (multiple unique hits) | ESTs (oviduct and | Promoter | Yes | BM440655; CN231463 |
| | | | testis) | | | |
| TP97/33 | HS4 BP | Chr9: 2,454,254 | UDP-glucose | Promoter | Yes | NM_001025777.1 |
| | | | ceramide glucosy like | | | (human) |
| | | | 1 homolog | | | |
| TP103/19 | HS4 BGBP | Chr6: 11,740,309 | Solute carrier family | Promoter | Yes | NM_152707.2 (human) |
| | | | 25 member 16 | | | |
| | | | homolog | | | |
| TP103/38 | HS4 BGBP | Chr8: 24,797,493 | ESTs (PGC and testis) | Intron 1 | Yes | C0769951 |
| | | | | Exon 1 | | DR419065 |
| TP104/48 | HS4 BGBP | Chr1: 61,953,438 | Transmembrane and | Intron 15 | No | NM 175861.2 |
| | | | tetratricopept repeat | | | (human) |
| | | | containing 1 homolog | | | |
| TP112/4 | HS4 CGCP | Chr4: 10,145,183 | EST (gonad) | Intron I | No | DT659505 |
| TP112/8 | HS4 CGCP | Chr12: 10,967,325 | zinc finger X-linked | Promoter | Yes | NM 007156.3, |
| | | | duplicated | | | NM 007157.3, |
| | | | A (ZXDA), | | | NM 025112.4 and |
| | | | ZXDB, or ZXDC | | | NM_001040653.1 |
| | | | homolog | | | (Human) |
| TP112/11 | HS4 CGCP | ChrZ: 72,904,646. | None (near LINE | | No | |
| | | | repeat) | | | |
| TP112/21 | HS4 CGCP | Chr8: 4,203,691 | Notch2 | Intron 1 | Yes | NM_024408.2 (human) |
| TP112/44 | HS4 CGCP | ChrUn: 25,214,782. | None (near satellite | | Yes | |
| | | | repeat) | | | |
| TP114/7 | HS4 CGCP | Chr3: 16,791,492 | None | | Yes | |
| TPI14/35 | HS4 CGCP | Chr1: 169,944,410 | None (LINE repeat) | | No | |
| DOCI | HS4 loxPCGCN | ND (multiple unique hits) | ND | | No | |
| DOC2 | HS4 loxPCGCN | Chr21: 1,818,407 | Protein kinase C zeta | Promoter | Yes | NM 002744 (human |
| | | | homolog and EST | Promoter | | Prkcz); CN237692 |
| | | | (testis) | | | (EST) |
| DOC3 | HS4 loxPCGCN | ChrUn: 29,847,677 | ESTs (testis) | Promoter | Yes | C0773920; C0772454 |
| DOC4 | HS4 loxPCGCN | No hits | ND | | Yes | |
| DOGS | HS4 loxPCGCN | Chr4: 70,149,012 | EST (embryo) | Intron 1 | Yes | BX933586 |
| DOC6 | HS4 loxPCGCN | Chr3: 56,244,635 | EST (pituitary and | Promoter | Yes | 00420386 |
| | | | hypothalamus) | | | |
| DOC7 | HS4 loxPCGCN | Chr8: 5,252,310 | SCYL1-like 3 | Exon 1 | Yes | NM 001012595.1 |
| DOC8 | HS4 loxPCGCN | Chr10: 4,731,544 | isocitrate | Promoter | Yes | NM_005530.2 (human) |
| | | | dehydrogenase 3 | | | |
| | | | (NAD+) alpha | | | |
| DOCIO | HS4 loxPCGCN | Chr14: 14,812,671 | Noggin homolog | Promoter | Yes | NM 001039604 |
| DOCI3 | HS4 loxPCGCN | Chr24: 5,764,120 | EST (pituitary, | Exon 1 | Yes | BI391861 |
| | | | hypothalamus, pineal) | | | |
| DOC17 | HS4 loxPCGCN | No hits | ESTs (embryo, | Exon or 3′ | Yes | CR523327, BX932892, |
| | | | muscle, brain) | | | BX934965 |
| DOC21 | HS4 loxPCGCN | Chr7: 23,302,240 | RNA binding motif, | Intron 2 | No | NM 205024.1 |
| | | | single stranded | | | |
| | | | interacting protein 1 | | | |
| DOC26 | HS4 loxPCGCN | Chr3: 78,546,391 | Proline-rich nuclear | Intron 1 | Yes | NM 001012291.1 |
| | | | receptor coactivator 1 | | | |
| DOC28 | HS4 loxPCGCN | Chr26: 3,781,825 | Synaptonemal | Promoter | Yes | NM_003176.2 (human) |
| | | | complex protein 1 | | | |
| DOC29 | HS4 loxPCGCN | No hits | ND | | Yes | |
| DOC30 | HS4 loxPCGCN | Chr3: 83,370,785 | SUMOI/sentrin | Intron 1 | Yes | NM_015571.1 (human) |
| | | | specific | | | |
| | | | protease 6 | | | |
| DOC31 | HS4 loxPCGCN | Chr5: 60,554,646 | Liver glycogen | Exon I | Yes | NM 204392.1 |
| | | | phosphorylase | | | |
| DOC33 | HS4 loxPCGCN | Chr2: 110,491,134 | None | | Yes | |

### EXAMPLE 22

### Efficient Integration with phiC31 Integrase

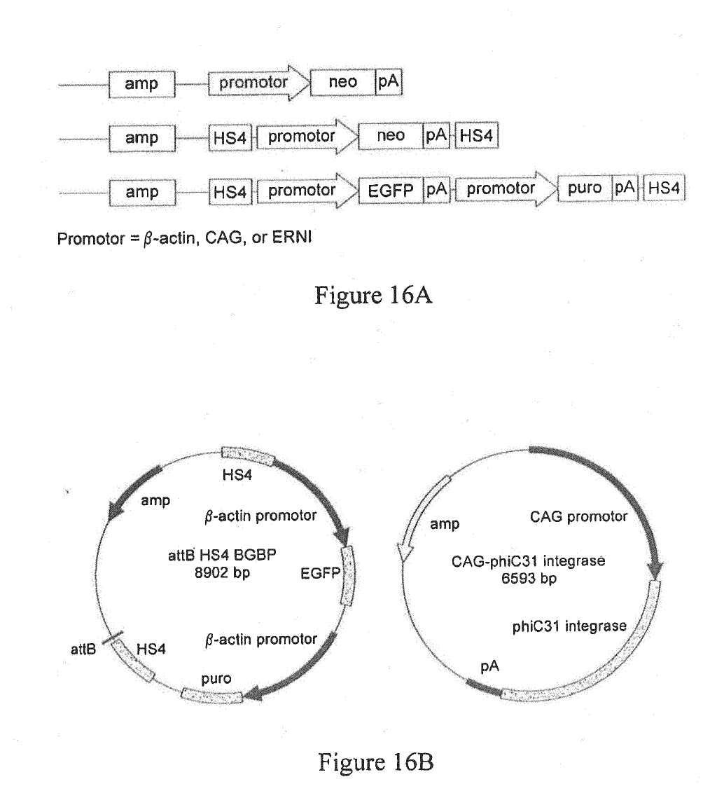

We have also used the phiC31 integrase system, which catalyzes site-specific recombination between an attB site and an attP site to insert foreign DNA into the chicken genome. Recombination between phiC31 attB and attP sites is irreversible, so that insertion of a circular construct bearing an attB site into the genome is stable and does not get looped out, even in the continued presence of integrase. In Xenopus (Allen and Weeks 2005 Nature Methods 2, 975-9.Transgenic Xenopus laevis embryos can be generated using phiC31 integrase.), mouse (Olivares et al. 2002 Nature Biotechnology 20, 1124-8. Site-specific genomic integration produces therapeutic Factor IX levels in mice; Belteki et al 2003 Nature Biotechnology 21,321-4. Site-specific cassette exchange and germline transmission with mouse ES cells expressing phiC31 integrase) and human cells (Groth et al 2000 Proc Natl Acad Sci USA. 97,5995-6000. A phage integrase directs efficient site-specific integration in human cells; Thyagarajan et al 2001, Mol Cell Biol. 21, 3926-34. Site-specific genomic integration in mammalian cells mediated by phagephiC31 integrase), it has been shown that phiC31 integrase can mediate integration of attB-containing plasmids into the unmodified genome, indicating that the genomes of these species contain pseudo-attP sites with sufficient sequence homology to the bacterial attP site to be recognized by the integrase. It has also been shown that the incoming plasmid must carry an attB site rather than an attP site for efficient integration (Belteki et al 2003; Thyagarajan 2001). An attB site was added to insulated HS4 p-actin EGFP β-actin puro (HS4 BGBP) construct, resulting in attB HS4 BGBP. Referring to the left panel of FIG. 16B, the integrase construct used in this study is shown to include the att-B containing plasmid in which the att-B site was added to the HS4 β-actin EGFP β-actin puro construct. Referring to the right panel of FIG. 16B, the plasmid used to express the integrase in cells from the CAG promoter is shown. Two versions of the integrase were made, one with an SV40 nuclear localization signal and one without. The attB HS4 BGBP and CAG-integrase plasmid DNAs were co-transfected as circular plasmids into PGCs. A large increase in colony formation was observed as compared to the non-integrase, linearized HS4 BGBP, from approximately 0.3 colony per 10⁶ cells with 20 ug of linear DNA to 5-10 colonies per 10⁶ cells with only 5 ug of DNA, representing a greater than 20-fold increase in colonies per DNA. The NLS version of the integrase yielded slightly fewer colonies compared to the non-NLS version. These data suggested that the integrase can recognize pseudo attP sites in the chicken genome, and that it can be used for efficient stable transfection in PGCs.

### EXAMPLE 23

### Identification of Insertion Site for Integrase Clones

The increase in efficiency of stable transfection using the integrase and attB-containing plasmids suggested that the chicken genome contains pseudo attP sites that can be recognized by the phiC31 integrase. In order to prove that the attB HS4 BGBP plasmid integrated via an integrase-mediated reaction, and not by random breakage of the vector, Southern blot analysis of genomic DNA from 5 independent PGC clones was performed and the intact, full-length transgene was observed in each case, with a structure consistent with integration via the attB site (data not shown). To further characterize the recombination breakpoints at the nucleotide level, to identify the pseudo attP sites, and to identify the chromosomal locations of the insertions, the junctions between the vector and the genomic insertion sites in 12 of our integrase PGC lines were cloned and sequenced. Plasmid rescue was performed as above for the non-integrase lines. We observed a dramatic decrease in the efficiency of cloning the junction fragments; the number of E. coli colonies obtained went from an average of 69 colonies per transformation for the non-integrase PGC lines to 3.1 colonies from integrase-mediated PGC lines per transformation. The reason for this is unclear but one possibility is that the repetitive DNA flanking the integrase clones (see below) was more difficult to digest with restriction enzymes. Plasmid DNA was purified from the colonies, sequenced and the attL and flanking sequences were determined (see Table 9 below). Since the genomic DNA had been digested with an enzyme that cuts within the transgene, only the flanking genomic DNA adjacent to the amp gene on the vector backbone could be identified with this method; the flanking DNA on the other side of the transgene insertion was not analyzed.

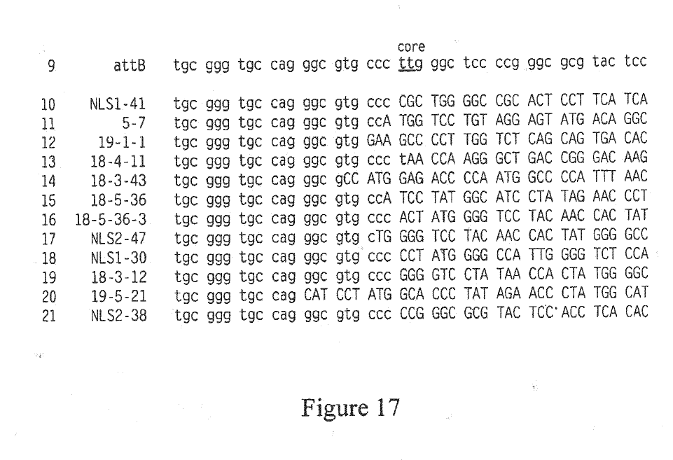

Referring to FIG. 17, the junctions between the attB plasmid and genomic sequences in the PGC clones derived from integrase-mediated transfection are shown. On the top line is the wild type attB site, with the core TTG which is normally the recombination crossover point is underlined SEQ ID No: 9, and below are the attL sequences from the integrase-mediated insertions SEQ ID Nos: 10-21. To determine where the splice occurred between the attB on the plasmid and the pseudo attP site in the genome, the PGC sequences were compared to attB SEQ ID No: 9. In the PGC sequences, the attB sequences donated by the plasmid are in lower case, and the genomic pseudo attP sequences are in upper case and bold.

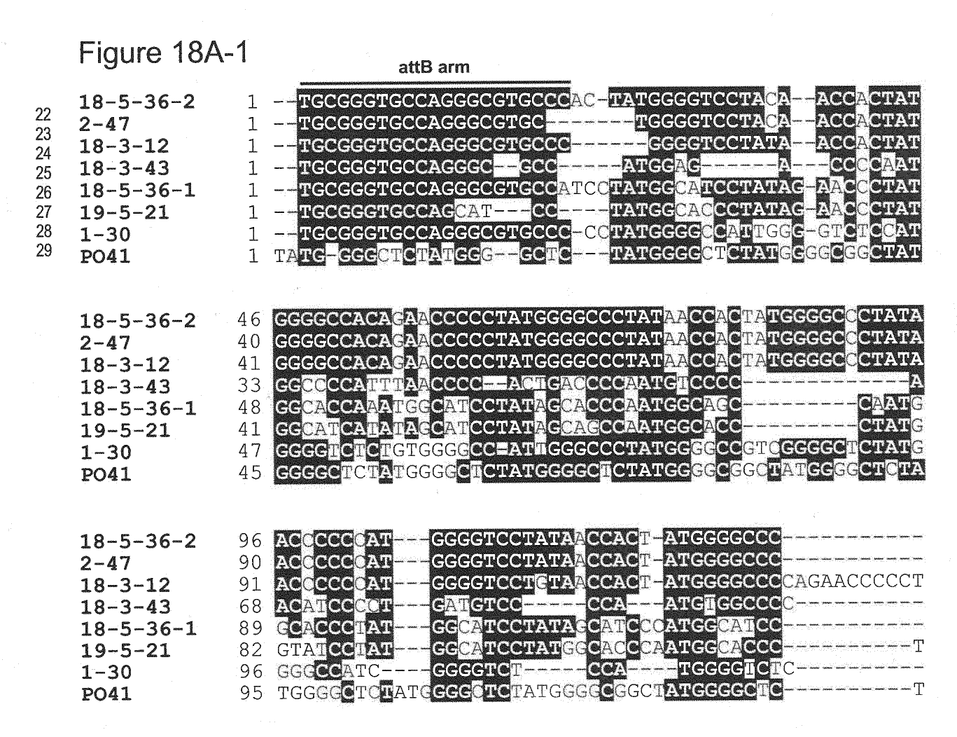

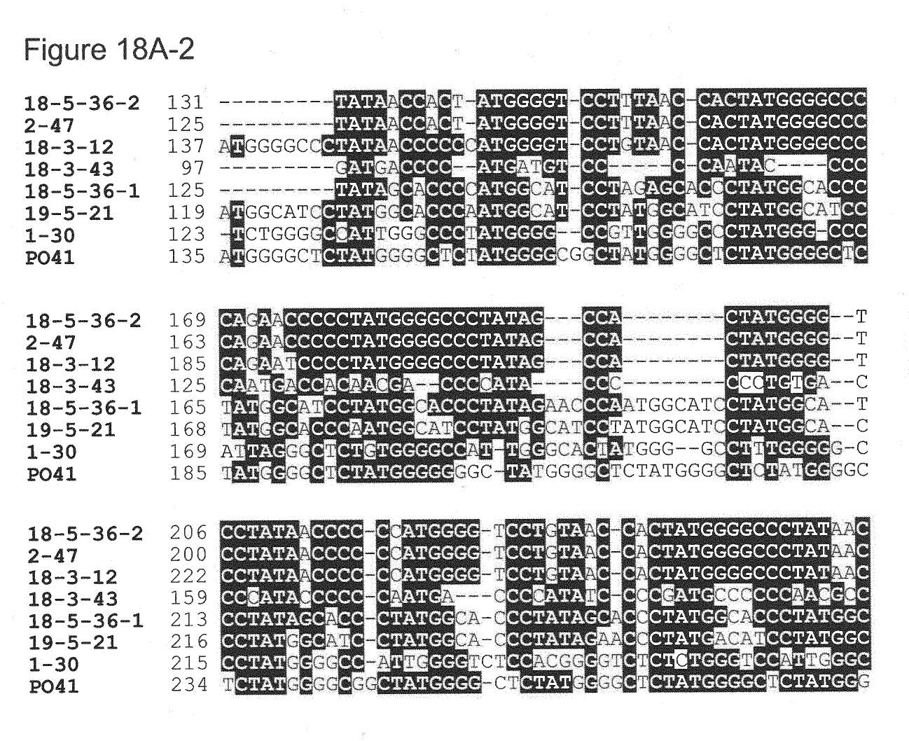

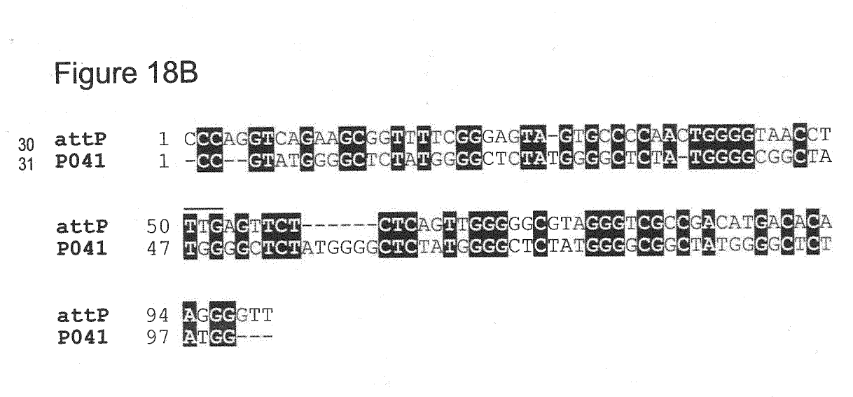

An attL sequence composed of about half of the attB sequence on the plasmid was found in each case joined to a pseudo attP site within the genome, suggesting that the integrase mediated the recombination reaction. Recombination between the plasmid-borne attB and the genome was not precise and did not usually take place at the core TTG nucleotides of attB. BLAT and BLAST searches mapped the genomic locations of each of the insertions. Strikingly, seven out of the eleven insertions that could be mapped occurred in repetitive DNA sequences. Using the RepeatMasker Web Server (Institute for Systems Biology) and ClustalW sequence alignment, repeats were analyzed and it was found that the sequences could be classified as the previously identified PO41 repeat (Wicker et al). Referring to FIG. 18A-1 and FIG. 18A-2, the alignment of the PCM 1-like repeats from PGC insertion sites with the P041 consensus sequence SEQ ID No: 29are shown. The PGC flanking sequences from all of the clones inserted into PO41-like repeats were aligned with each other and with the P041 consensus (Wicker et al. 2004) The first 20 nucleotides are the attB sequence donated by the vector (as indicated above the alignment), followed by the genomic flanking sequence from each clone. Nucleotides shared by more than half of the sequences are boxed in black. Alignments of the sequences indicated that two of the PGC lines (2-47 SEQ ID No: 23 and 18-5-36-2 SEQ ID No: 22) carried insertions of attB HS4 BGBP at the same genomic site. The two insertions are independent, as shown by the fact that the nucleotide sequence at the attB-pseudo attP junction is different between the two. Another sequence (18-3-12 SEQ ID No: 24) was identical to the first two except for a 20 bp insertion. None of the P041 repeats identified in the insertions were exact copies of the consensus sequence, and the level of sequence homology between the P041 consensus and the insertion site repeats ranged from 47% (18-3-43) to 77% (1-30) nucleotide identity. Referring to FIG. 18B, alignment of 100 bp of the P041 consensus repeat with 100 bp of attP revealed about 46% sequence identity, which is higher than that observed for pseudo attP sites in human cells (Thyagarajan 2001). The P041 repeat is thought to be present in 259 locations in the genome (Wicker et al), with each location consisting of several kilobases of the 41 bp repeating unit. Several of the flanking genomic fragments were in the 10-12 kb size range; when the two ends of these fragments were sequenced, both were repetitive, suggesting that the overall size of the repeat was large. Thus, P041 sequences represent a large, preferred target in the chicken genome for the phiC31 integrase.

The remaining 4 integrase-mediated insertions were in unique DNA sequences. One of the sequences (19-1-1 SEQ ID No: 12) was in an intergenic region of unique sequence on chromosome 21, and one (1-41 SEQ ID No: 10) was inserted in the promoter region of the chicken ortholoque of the Wilm's tumor (WT1) gene on chromosome 5. One sequence (5-7 SEQ ID No: 11) was on chromosome 1 in multiple places, which could represent a local gene family or low-copy number repeat. One sequence (18-4-11 SEQ ID No: 13) did not match the extant sequences in either the chicken genome or the general ‘non-redundant’ databases, and thus is likely in a region of the genome that has not yet been sequenced. One final insertion (2-38 SEQ ID No: 21) yielded only a very short sequence consisting of the pseudo attP site, which could not be identified in the database.

TABLE 9
PGCs with integrase-mediated constructs

| | | Chromosomal | | | | Accession |
|---|---|---|---|---|---|---|
| Clone | Construct | location | Gene | Position | CpG Island? | Number |
| Int5/7 | attB HS4 BGBP | Chrl (multiple | None | | No | |
| | | unique hits) | | | | |
| NLS1/41 | attB HS4 BGBP | Chr5: 5,530,594 | Wilm's tumor | Exon lb | Yes | NM 205216.1 |
| | | | suppressor gene | | | |
| | | | WTI | | | |
| Int19/1-1 | attB HS4 BGBP | Chr21: 3,167,543 | None | | No | |
| Intl 8/4-11 | attB HS4 BGBP | No hits | ND | | Yes | |
| Intl 8/3-43 | attB HS4 BGBP | ND | P041-like repeat | | No | |
| Intl 8/5-36 | attB HS4 BGBP | ND | P041-like repeat | | No | |
| Intl 9/5-21 | attB HS4 BGBP | ND | P041-like repeat | | No | |
| Intl 8/5-36-2 | attB HS4 BGBP | ND | P041-like repeat | | No | |
| Intl 8/3-12 | attB HS4 BGBP | ND | P041-like repeat | | No | |
| NLS 1/30 | attB HS4 BGBP | ND | P041-like repeat | | No | |
| NLS2/47 | attB HS4 BGBP | ND | P041-like repeat | | No | |

### EXAMPLE 23

### Identification of the Genetically Modified Chromosomes

We also noted the chromosome in which insertion occurred for those lines that contained insertions in unique sequences for which we could assign a location. Among 28 independent insertions in PGCs, we observed insertions in 17 different chromosomes (Tables 8 and 9), out of the 38 chromosomes in the chicken karyotype. About half of the insertions are in the macrochromosomes (chromosomes 1-6; 13 insertions) and the other half in the microchromosomes (chromosomes 7-38; 14 insertions), with one insertion in the Z chromosome. The ratio of insertions into macrochromosomes and microchromosomes is proportional to their physical contribution to the genome, indicating that there is no regional bias for integration.

### EXAMPLE 24

### Gene Targeting

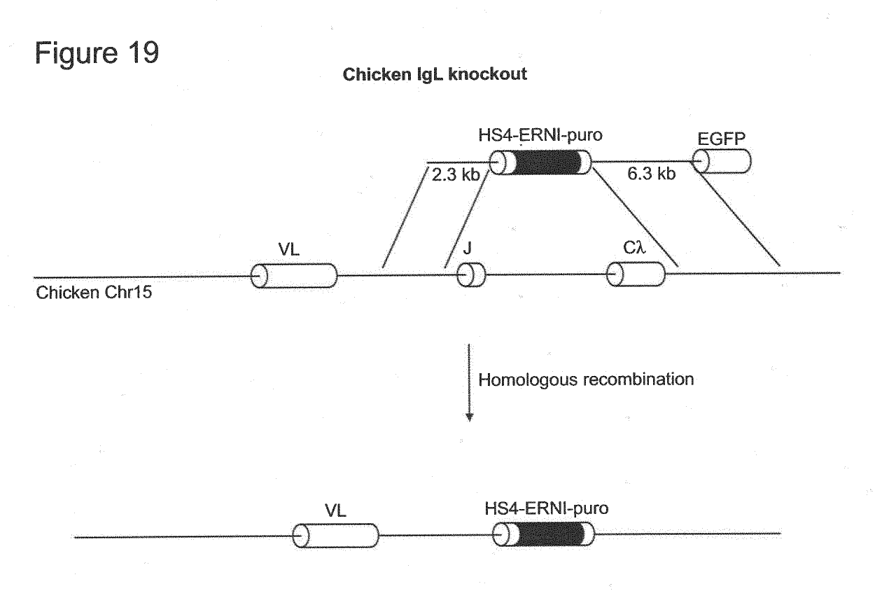

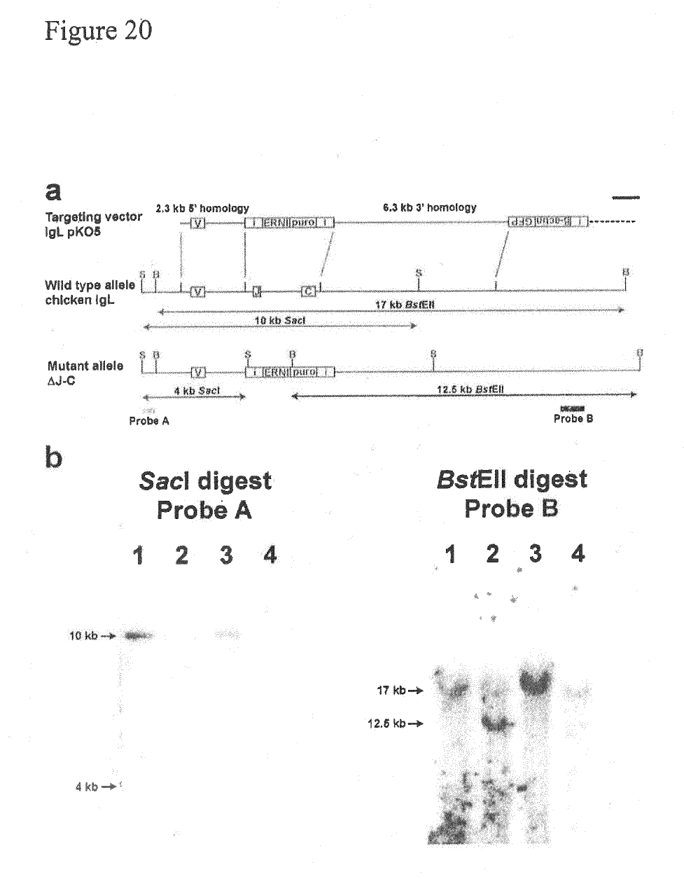

A targeting vector was designed to replace the J and C regions of the immunoglobulin light chain gene with an HS4 ERNI-puro selection cassette when inserted into the endogenous locus by homologous recombination (FIG. 19). As noted above, the ERNI promoter (driving the puromycin cassette) is specifically expressed in early embryos (Acloque et al 2001, supra, and was expected to yield drug-resistant colonies in PGCs at frequencies similar to the other promoters. Referring to FIG. 20A, the top line is a diagram of the targeting vector for the chicken IgL gene, IgL K05. It is designed to replace the 2.3 kb) J-C region of the IgL gene with a 3.1 kB HS4 ERNI-puro selectable marker (shown as I and HS4 insulator). The two homology arms are 2.3 and 6.3 kB in length. At the 3′ end a β-actin EGFP allows for screening puro-resistant clones for green fluorescence to enrich for targeted clones. The dashed line at the end is the pKO vector backbone (Stratagene). On the middle line is a diagram of the wild type allele of the germline configuration of the IgL gene, with the single variable (V), joining (J) and constant (C) region genes. The restriction sites used for Southern analysis of targeted clones are shown (S, SacI; B, BstEII) and the wild type fragment sizes with double arrowheads shown below. On the lower line is the structure of the mutant allele in which the J and C regions have been deleted and replaced with the HS4 ERNI-puro. The restriction map is shown, with the mutant fragment sizes shown below. The probes used in Southern analysis were both external to the targeting vector and their positions are shown.

Four clones were isolated from 21 transfections, using a total of 1.05×10⁸ cells and 210 μg of linearized DNA. Two of the clones expressed GFP indicating that they had integrated randomly in the genome and retained the GFP gene. Southern blot analysis of the 4 clones showed that one of the non-green clones (clone 2) was heterozygous for the targeted mutation, using probes from both sides of the integration. Referring to FIG. 20B, the four puromycin-resistant clones were analyzed by Southern analysis. Clones 1 and 2 were not-green whereas clones 3 and 4 expressed GFP. On the left panel, genomic DNA from the PGC clones was digested with SacI and hybridized with probe A to analyze targeting on the 5′ side of the IgL gene. On the right panel, DNA was digested with BstEU and hybridized with probe B for targeting on the 3′ side of the IgL gene. Clone 2 showed the expected sized fragments for a heterozygous, targeted clone.

### EXAMPLE 25

### GO Chimeras Carrying the J-C Knock Out Vector

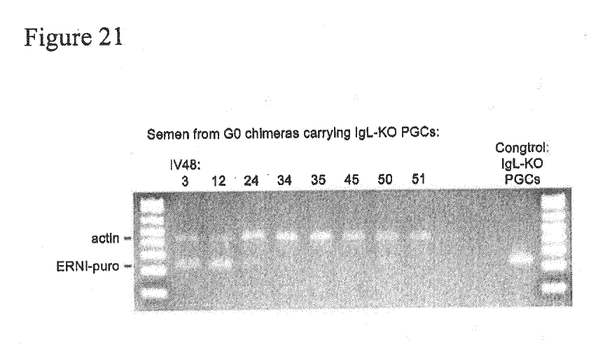

PGCs carrying the J-C knock-out described in Example 24 were injected into the vasculature of Stage 13-15 (H&H) White Leghorn recipient embryos. Phenotypically, the hatched chicks resembled White Leghorns. Males were reared to sexual maturity and semen was collected by abdominal massage. A PCR analysis using forward primers ERNI-133F: 5′-TTGCTCAAGCCCCCAGGAATGTCA-3′ SEQ ID No: 32 and reverse primers Puro-8R: 5′-CGAGGCGCACCGTGGGCTTGTA-3′ SEQ ID No: 33 is shown in FIG. 21. Referring to FIG. 21, amplified DNA of the expected 248 bp size was present in semen from at least two of the GO chimeras indicating that the genetically modified primordial germ cells had entered the germline.

### EXAMPLE 26

### Insertion of 13-Actin-Neo Into the Aldehyde Dehydrogenase Locus

PGCs from parental cell line 13 were grown and 5×10⁶ cells in 400 μl were electroporated with 20 μg of a linearized B-actin-neo construct, using an exponential decay pulse of 198V and 9000 μF and plated into 48-lcm² wells to obtain single clones. The cells were grown in the presence of neomycin resulting in the growth of neomycin resistant clones that were transferred to new wells to be expanded. The cells were analyzed by Southern analysis to establish stable integration of the transgene and sequencing showed that the construct was integrated in chromosome 19 in the promoter region of the aldehyde dehydrogenase gene, an enzyme involved in aldehyde metabolism.

PGCs carrying the β-actin-neo construct were grown and injected into Stage 1316 (H&H) recipient embryos. The embryos were hatched and 4 roosters were grown to sexual maturity and tested for germline transmission. The germline transmission rate of the roosters was 0, 0, 0.5 and 0.5%, respectively. Heterozygous offspring from one of these roosters were grown to sexual maturity and mated to obtain homozygous offspring.

TABLE 10
Offspring from breeding heterozygous chickens carrying 13-actin-neo
(BN).

| | | Genotype all offspring | | | dead & died <1 day | | |
|---|---|---|---|---|---|---|---|
| rooster | hen | w/w | tg/w | tg/tg | w/w | tg/w | tg/tg |
| BN-16 | BN- | 4 | 16 | 7 | 1 | 3 | 2 |
| | 09 | | | | | | |
| BN-34 | BN- | 0 | 2 | 2 | 0 | 0 | 0 |
| | 27 | | | | | | |
| BN-20 | BN- | 5 | 15 | 3 | 1 | 3 | 0 |
| | 31 | | | | | | |
| BN-39 | BN- | 1 | 11 | 3 | 0 | 3 | 0 |
| | 35 | | | | | | |
| BN-36 | BN- | 0 | 0 | 2 | | | |
| | 13 | 10 | 46 | 17 | 2 | 9 | 2 |
| | | (14%) | (63%) | (23%) | (20%) | (20%) | (12%) |

w/w: wild type,
w/tg: heterozygous bird,
tg/tg: homozygous bird

Five breeding pairs of heterozygous roosters and hens produced a total of 73 chicks that were evaluated for the presence of the BN transgene. A total of 10 chicks (14%) were wild type, 46 chicks (63%) were heterozygous and 17 chicks (23%) were homozygous (Table 10). This distribution is not significantly (P>0.01) different from the distribution of 18.25/36.5/18.25 expected from Mendelian segregation (Chi-square=6.55). The proportion of chicks that died around hatch were similar among the genotypes. These results indicate that the insertion of the BN transgene did not induce a lethal phenotype.

The homozygous roosters were grown to sexual maturity to test for fertility. Five roosters were bred to wild type hens and fertility, embryonic death and hatching percentages were calculated (Table 11). Although the fertility of 2 roosters was relatively low, the semen production of these birds was poor and therefore the number of sperm per insemination was also low. The fertility of two of the birds was very good (>90%) and the fertility of one bird was intermediate. The hatchability of fertile eggs from all birds was within the normal range. Taken together, these data indicate that the reproductive function of BN/BN birds was normal.

TABLE 11
Fertility of homozygous BN rooster

| Rooster | # eggs | infertile | | early death | # hatched | % hatched |
|---|---|---|---|---|---|---|
| BN104 | 185 | 49 | 74% | 11 | 112 | 90% |
| BN113 | 110 | 5 | 95% | 3 | 93 | 91% |
| BN128 | 74 | 44 | 41% | 1 | 24 | 83% |
| BN135 | 85 | 39 | 54% | 5 | 32 | 78% |
| BN137 | 188 | 5 | 97% | 8 | 158 | 90% |

Since the BN transgene was integrated into the aldehyde dehydrogenase gene and no effect was seen on the viability of homozygous birds, we evaluated transcription of the aldehyde dehdrogenase message.
mRNA was prepared from blood from two BN/BN homozygous birds, one BN/+ heterozygous bird and one wild-type bird (+/+) with Oligotex Direct mRNA Kit (Qiagen). cDNA was then synthesized from 5 ml RNA using the Thermo-Script RT-PCR system for First Strand cDNA Synthesis (Invitrogen). 1 ml of cDNA was used in the subsequent PCR reaction using the following primers:

ALDH3A2-3
SEQ ID No: 34
AGTGGTCACCGGGGGAGT
ALDH3A2-4
SEQ ID No: 35
TCACAGACACAATGGGCAGG
Actin RT-1
SEQ ID No: 36
AAC ACC CCA GCC ATG TAT GTA
Actin RT-2
SEQ ID No: 37
TTT CAT TGT GCT AGG TGC CA

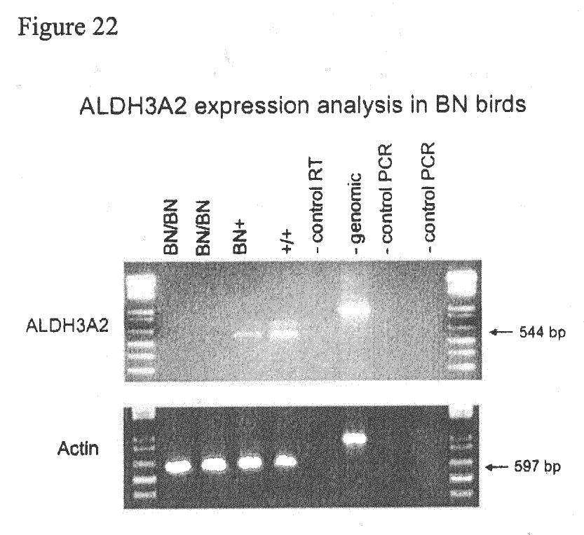

ALDH3A2-3 and A2-4 primers SEQ ID Nos: 34, 35 amplified a 544 and a 680 bp PCR product for the aldehyde dehydrogenase 3 family member A2 transcript. Actin RT-1 and RT-2 primers SEQ ID Nos: 36, 37were used to amplify a 597 bp PCR product for the Actin transcript. As shown in FIG. 22 aldehyde dehydrogenase 3 family member A2 transcripts were detected in the heterozygous (BN/+) and wild-type bird (+/+), but not in the homozygous BN bird (BN/BN) indicating that the insertion of the βactin-neo transgene generated an insertional knock-out of the aldehyde dehydrogenase 3 gene that does not have a morphological phenotype.

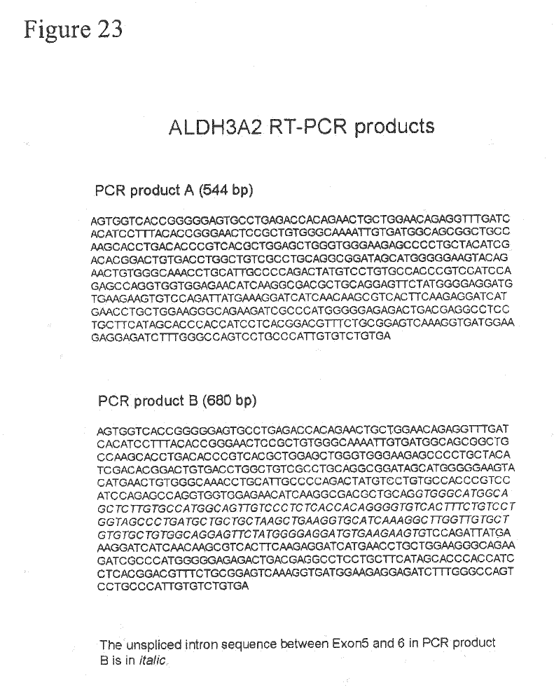

Confirmation that the primers amplified the aldehyde dehydrogenase 3 family member A2 transcript was obtained by sequencing the 544 bp and 680 bp PCR products. The 544 bp product is entirely contained within the 680 bp PCR product which also contains an unspliced intron of 136 bp between exon 5 and 6 (FIG. 23). Comparison of these sequences with those of the published chicken genome showed that they are identical.

### EXAMPLE 27

### Insertion of β-Actin-gfp-bβ-Actin-Puro Into an Unknown EST

PGCs from parental cell line 54 were grown and 5×10⁶ cells were electroporated with 20 ng of a linearized β-actin-GFP-β-actin-puro construct and plated into 48-lcm² wells to obtain single clones. The cells were grown in the presence of puromycin resulting in the growth of only puromycin resistant clones. Resistant clones were transferred to new wells to be expanded. The cells were analyzed by Southern analysis to establish stable integration of the transgene and sequencing showed that the construct was integrated in chromosome 8 in a novel gene (EST C0769951).

PGCs construct were grown and injected into the vasculature of Stage 13-16 (H)H recipient embryos. The embryos were hatched and 8 roosters were grown to sexual maturity at tested for germline transmission. The germline transmission rate of the roosters was 0, 1, 11, 12, 13, 16, 28 and 92%, respectively. Heterozygous offspring from these roosters was grown to sexual maturity and mated to obtain homozygous offspring (Table 12).

TABLE 12
Offspring from breeding heterozygous chickens carrying Pactin-GFP-βactin-puro (BGBP).

| | | | | | | | | died before | | |
|---|---|---|---|---|---|---|---|---|---|---|
| | | genotype all offspring | | | dead & died < Iday | | | sexual maturity | | |
| rooster | hen | w/w | tg/w | tg/tg | w/w | tg/w | tg/tg | w/w | tg/w | tg/tg |
| 34-09-06 | 34-62-46 | 15 | 21 | 13 | 4 | 11 | 11 | — | 2 | 2 |
| 34-47-01 | 35-08-03 | 8 | 11 | 12 | 5 | 2 | 10 | — | 0 | 2 |
| 34-62-18 | 35-60-20 | 13 | 15 | 12 | 4 | 1 | 10 | — | 0 | 2 |
| 34-62-29 | 35-60-06 | 13 | 25 | 11 | 3 | 5 | 10 | — | 2 | 1 |
| 35-08-01 | 34-62-13 | 10 | 17 | 14 | 4 | 2 | 8 | — | 0 | 6 |
| 35-60-01 | 35-08-07 | 14 | 20 | 7 | 2 | 3 | 4 | — | 1 | 3 |
| 35-60-18 | 35-08-06 | 16 | 17 | 7 | 5 | 6 | 7 | — | 0 | |
| 34-62-18 | 35-60-11 | 1 | 2 | 4 | 1 | — | 4 | — | — | 0 |
| | | 90 | 128 | 80 | 27 | 30 | 64 | | 4 | 16 |
| | | (30%) | (46%) | (25%) | (30%) | (23%) | (80%) | | (3%) | (20%) |

w/w: wild type,
w/tg: heterozygous bird,
tg/tg: homozygous bird

Eight breeding pairs of heterozygous roosters and hens produced a total of 298 chicks that were evaluated for the presence of the BGBP transgene. A total of 90 chicks (30%) were wild type, 128 chicks (46%) were heterozygous and 80 chicks (25%) were homozygous. These results conform to the expectations of 25% wild type, 50% heterozygous and 25% homozygous offspring indicating the transgene was inherited in a Mendelian function. The majority of the homozygous offspring died at or around hatch with 95% of all chicks dead by 6 weeks of age. These results indicate that the insertion of the BGBP transgene created a functional knock-out of a gene essential for viability.

To bypass this problem, it is advantageous to insert transgenes into a predetermined location rather than uncontrolled, random insertion. The ability to insert transgenes into a known location in the genome has further potential advantages over random insertion. Transgenes inserted for the purpose of over-expression of a protein product can be inserted into a location that is known to permit high levels of expression and does not undergo silencing by encroaching heterochromatin. Furthermore, the insertion of the transgene can be predicted to cause no harmful effects to the animal or cell line, either in the heterozygous or homozygous state. It thus becomes unnecessary to screen large numbers of different random insertions to find one with high levels of expression and that does not interrupt an important endogenous gene.

### EXAMPLE 28

### Creation of Transgenic Birds Carrying a Conditional Apoptosis-Inducing Gene (Reaper)

### Design of the Reaper Transgene

The Reaper transgene (loxP-stop-loxP-Reaper construct) was generated as follows. The Reaper cDNA was cloned by RT-PCR using D. melanogaster embryo poly (A)+ MRNA and REAPER F1 (CAC CAG AAC AAA GTG AAC GA SEQ ID No: 38) and REAPER F2 (TGT TTG ACA AAA AAT TGA TGC) primers SEQ ID No: 39. The Reaper cDNA was inserted into the RI site of the CX-backbone generating the CX-Reaper construct. A Kpnl site was inserted into the Reaper cDNA 3′ prime of the start codon by site directed mutagenesis. A 1.5 kb loxP-stop-loxP cassette from pBS302 (Gibco/BRL) was cloned into the Kpnl site to generate the CX-LoxP-stop-loxP-Reaper. The loxP-stop-loxP-Reaper fragment was inserted into the pENTRB2 clone (Invitrogen) using the RI and Notl sites. The loxP-stop-loxP-Reaper fragment was then recombined into the pLenti6/UbC/V5-DEST (pLenti Gateway vector Invitrogen) creating the UbC-loxP-stop-loxP-Reaper construct.

### Production of Transgenic Birds Using the ViraPower Lentiviral Expression System

To generate the lentivirus carrying the loxP-stop-loxP-Reaper transgene, the ViraPower lentiviral expression system (Invitrogen) was used and high lentiviral titers up to 4.8×109 cfu/ml were generated. For virus production 293T cells were co-transfected with the UbC-loxP-stop-loxP-Reaper construct and a virapower packaging mix including a VSV-G encoding plasmid using Lipofectamine. The viral supernatant was collected 24 hours after transfection and was concentrated by centrifugation. The viral titer was determined by transducing HT1080 cells with the viral supernatant. High titers were produced to ensure high transduction and germline transmission efficiency.

To infect chicken embryos with the virus 1.5 ul of concentrated viral solution was injected into the subgerminal cavity of stage X embryos. After incubation at 37.5-38° C. for 3 days the embryos were transferred to a second surrogate shell and incubated until hatch at 37.5 to 38° C. and 50% humidity. In total 398 embryos were injected with the virus carrying the UbC-loxP-stop-loxP-Reaper transgene. 155 birds hatched and were analyzed for the presence of the transgene and gender by PCR on DNA isolated from comb tissue. 13 male chicks were positive for the UbC-loxP-stop-loxP-Reaper transgene. 10 of those were also positive for the UbC-loxP-stop-loxP-Reaper transgene by PCR on DNA isolated from semen. 3 males (6-03, 6-51 and 9-51 G0 founder males) transmitted the transgene to the next generation. The transmission frequencies for 6-03, 6-51 and 9-51 were 0.32%, 0.26% and 0.16% respectively (Table 13).

TABLE 13
Germline transmission frequencies for the
6-03, 6-51 and 9-51 GO roosters.

| | # total | # male | # female | # total | |
|---|---|---|---|---|---|
| Rooster | pos. alive | pos. | pos. | screened | % positive |
| 6-03 | 4 | 3 | 1 | 1236 | 0.32 |
| 6-51 | 4 | 2 | 2 | 1497 | 0.26 |
| 9-51 | 2 | 1 | 1 | 1206 | 0.16 |
| total | 10 | 6 | 4 | 3939 | 0.25 |

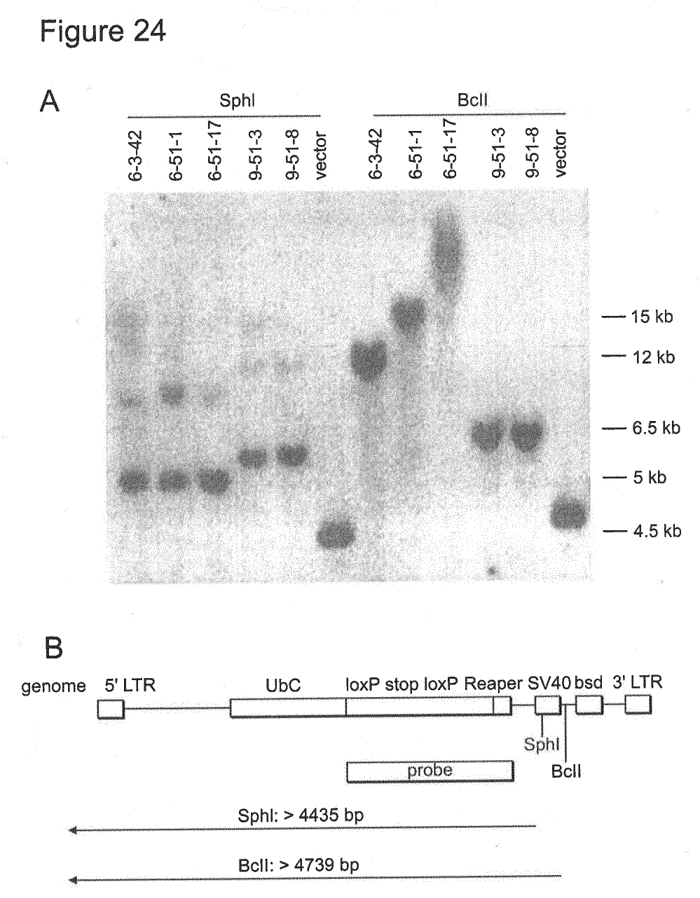

G0 founder males (6-03, 6-51 and 9-51, carrying different insertions), were bred to stock hens and their G1 offspring were analyzed for the presence of the transgene and the integration sites of the transgene. Genomic DNA from individual birds was analyzed by Southern blot analysis. Genomic samples and the UbC-loxP-stop-loxP-Reaper vector (control) were digested with Sphl or Bell. The digested DNA was separated on a 0.7% agarose gel, blotted to nylon membrane and probed with a radiolabeled Reaper specific probe to identify junction fragments. As shown in FIG. 24A, the sizes of the hybridizing genomic fragments were larger than the control indicating that the transgene was integrated. The hybridizing genomic fragments for the 6-03, 6-51 and 9-51 lines had different sizes indicating that 6-03, 6-51 and 9-51 are independent lines.

### EXAMPLE 29

### Creation of Chickens Carrying Cre-Recombinase

### Design and Assembly of Cre Transgene.

To express Cre recombinase in chickens, a transgene was built in which the Cre gene was placed under the transcriptional control of the chicken ERNI promoter. The ERNI gene (also known as cENS-1) is expressed in early chicken embryos (around stage X, the stage of the newly laid egg, when the embryo is a undifferentiated sheet of cells prior to gastrulation), and in neural tissue. The Cre transgene was thus designed to be expressed in early embryos where it would catalyze recombination of loxP sites of the loxP-Reaper transgenes or other loxP-containing transgenes placed in the genome. Since Cre would be expressed at an early stage, the resulting chicken that develops should carry recombined transgenes in every germ layer and every cell of its body.

To introduce the Cre transgene into the chicken germline, a Lentiviral vector approach was taken. A lentiviral transgene was constructed based on the Invitrogen pLenti6-V5 Dest Lentiviral vector. The Lentiviral vector elements of pLenti6-V5 Dest were combined with the ERNI-Cre gene to produce the pLenti-ERNI-Cre construct. Lentivirus was produced and used to infect early embryos, where it stably integrated into the genome. Approximately 20 transgenic founder birds were produced carrying the pLenti-ERNI-Cre transgene.

The chicken ERNI promoter was PCR amplified with the following primers; ERNI −738: 5′-ATGCGTCGACGTGGATGTTTATTAGGAAGC-3′ SEQ ID No: 40 ERNI +83: 5′-ATGCGCTAGCTGGCAGAGAACCCCT-3′ SEQ ID No: 41

The 822 bp PCR product was cloned into pGEM T-easy (Promega) and sequenced. The ERNI promoter was then released from the vector by digestion with SacII (subsequently blunted with T4 DNA polymerase) and Spel. The CMV promoter was removed from the lentiviral vector pLenti6 V5-Dest (Invitrogen) by digestion with Clal (subsequently blunted with T4 DNA polymerase) and Spel. The ERNI promoter was then ligated to the pLenti6 V5-Dest lentiviral vector backbone, replacing the CMV promoter therein with the ERNI promoter, resulting in pLenti-ERNI.

The Cre gene was PCR amplified with an SV40 nuclear localization sequence on the N-terminus and convenient restriction sites for cloning (Bglll on the 5′ end and EcoRI on the 3′ end) with the following primers:

Cre-C:
SEQ ID No: 42
5′-CCG CCG GAG ATC TTA ATG CCC AAG AAG AAG AGG AAG
CTG TCC AAT TTA CTG ACC GTA CAC-3′
Cre-R1:
SEQ ID No: 43
5′-TCGAATTCGAATCGCCATCTTCCAGCAGGCG-3′

The 1040 bp PCR product was digested with Bglll and EcoRI and gel purified. The shuttle vector pENTR 2B (Invitrogen) was digested with BamHI and EcoRI and the vector backbone was gel purified. The Cre PCR product was ligated to the pENTR 2B vector and clones obtained. Clones were sequenced to determine that the Cre gene was as expected and had not acquired any mutations during PCR amplification.

To recombine the Cre gene into the pLenti-ERNI construct and place it under the transcriptional control of the ERNI promoter, the LR clonase reaction (Invitrogen) was performed using the pENTR 2B-Cre clone as the source of the Cre gene and the pLenti-ERNI vector as the recipient. The final construct was thus obtained, pLenti-ERNI-Cre (8408 bp), which was used to produce lentivirus carrying the ERNI-Cre transgene.

Production of Transgenic Birds Carrying the pLenti-ERNI-Cre-Transgene

The pLenti-ERNI-Cre lentivirus was produced in 293FT cells. For each transfection into 293FT cells to produce lentivirus, 8 million 293FT cells at 75% confluency were transfected with 3 ug of circular pLenti-ERNI-Cre plasmid DNA using Lipofectamine reagent (Invitrogen). Virapower packaging mix (Invitrogen) was used to express the viral proteins necessary to make lentivirus in the 293FT cells. The cell culture supernatant containing lentivirus was harvested 2 days after transfection, filtered to remove cellular debris, and the lentivirus particles were concentrated by centrifugation at 48,000 g for 90 minutes. The viral pellet was resuspended in 1/200 the starting volume of culture supernatant and frozen at −80 C in 40 ul aliquots.

The infectious titer of each batch of lentivirus stock was determined on HT1080 cells by serially diluting the viral stock 10⁴ to 10⁻⁸ and adding to cultures of HT1080 with 1 ul polybrene. Two days after addition of the lentivirus, blasticidin selection (5 ug/ml) was initiated. Culture medium was replaced every two days as the cells died from blasticidin toxicity. Ten days after initiating blasticidin selection, colonies were analyzed for the presence of the transgene and gender by PCR on DNA isolated from comb tissue. 8 male chicks were positive for the Erni-Cre transgene. 13 males were also positive for the Erni-Cre transgene by PCR on DNA isolated from semen. 4 out of 6 males tested transmitted the Erni-Cre transgene to the next generation. The germline transmission frequency for the Erni-Cre G0 roosters varied between 0.24 and 1.32% (Table 14).

TABLE 14
Germline transmission frequencies for Erni-Cre GO roosters.

| | # total | # male | # female | # total | |
|---|---|---|---|---|---|
| Rooster | pos. alive | pos. | pos. | screened | % positive |
| 18-22 | 11 | 5 | 6 | 834 | 1.32 |
| 19-09 | 4 | 3 | 1 | 679 | 0.59 |
| 19-19 | 1 | 0 | 1 | 420 | 0.24 |
| 19-69 | 8 | 7 | 1 | 621 | 1.29 |
| total | 24 | 15 | 9 | 2554 | 0.94 |

Analysis of Transgenic Chickens Carrying the pLenti-ERNI-Cre Transgene

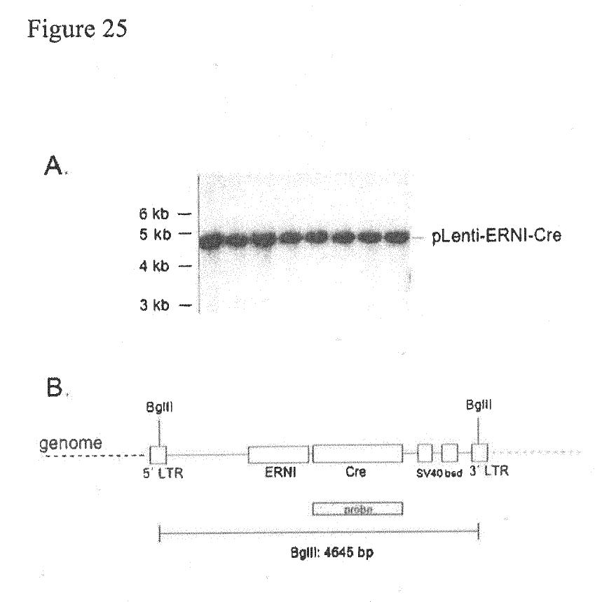

To verify that the pLenti-ERNI-Cre transgene integrated intact into the chicken genome, Southern blot analysis was used. Genomic DNA from chickens that had been identified first by PCR to carry the Cre transgene was extracted and digested with an enzyme that cuts in the viral 5′ and 3′ LTR sequences (Bglll) so that the full-length intact transgene would be observed. Birds with different, independent insertions of the transgene were chosen for analysis. Genomic DNA was digested with Bglll enzyme, transferred to nylon membrane, and probed with radiolabeled Cre gene. FIG. 25A shows a representative Southern of 8 ERNI-Cre lines. Of 11 lines tested, 10 contained the expected 4.6 kb ERNI-Cre transgene band, showing that the transgene integrated intact into the genome. One line had a smaller band, indicating a deletion or rearrangement of the transgene.

### EXAMPLE 30

### Establishment of Cells Lines Allowing Site-Specific Integration of a Transgene

There are two main ways to insert foreign DNA into a predetermined location into a host genome: homologous recombination (gene targeting) or site-specific recombination into a recognition site such as attP. Homologous recombination is inefficient in most vertebrate cell types and usually requires screening many clones to identify one or a few that have the desired insertion. Site-specific recombination is a high-fidelity, highly efficient process that can be used to insert foreign DNA into predetermined sites without screening a large number of clones. Site-specific insertion depends on the use of phiC31 integrase to insert an attB-containing construct into a unique attP site placed in the genome, or into a pseudo-attP site. To use an authentic attP site placed into the genome as a docking site, the attP site must be placed into a preferred, pre-determined location. The attP site is placed into such a preferred location in the genome by random insertion or by homologous recombination. If the recognition site is placed in the genome by random insertion, then the location of insertion must be validated to ensure that an important gene has not been disrupted. A recognition site placed into the genome then serves as the “docking site” for insertion of transgenes using phiC31 integrase.

To select for integrase-mediated transgene insertion specifically at the docking site, a selectable marker system is used to select for the correct insertion. The docking site is designed such that the attP site is adjacent to a drug selectable marker (such as the puromycin resistance gene) without a promoter. Cells carrying the docking site are thus sensitive to drug selection with puromycin. The transgene to be inserted into the docking site contains a promoter adjacent to its attB site, but no selectable marker. Insertion of the transgene into the docking site places the promoter upstream of the selectable marker, activating its transcription and conferring puromycin resistance. Insertion of the transgene into other locations in the genome do not lead to drug resistance and such insertions are eliminated by drug selection.

The attP docking site construct consists of an attP site placed adjacent to a promoter-less drug selectable marker, such as puromycin resistance. Since the puromycin resistance gene is not expressed, another selectable maker, such as the β-actin promoter driving the neomycin selectable marker, must also be included. An EGFP gene can also be included. Flanking each side of these elements are two copies of the β-globin HS4 insulator to insulate the construct from neighboring chromatin. For future removal of the β-actin neo and EGFP portions of the construct, loxP sites are placed flanking these elements. All of these portions of the construct serve as the vehicle for delivery of the authentic attP site into the genome. The order of DNA elements is: HS4; attP; promoterless puromycin resistance gene; loxP; p-actin or CAG promoter; EGFP; β-actin or CAG promoter; neomycin resistance gene; HS4; plasmid backbone (pBluescript). The construct is linearized and transfected into cultured PGCs, and drug resistant colonies are obtained. These colonies are expanded for further analysis.

Since it is important to know where in the genome the docking site is situated, the chromosomal insertion site of the docking site construct in each clone is determined. Flanking genomic DNA is obtained and sequenced and compared to the chicken genome database. The majority of the clones are found to insert in CpG islands, which are regions of the genome normally associated with promoter regions of genes, especially of housekeeping or ubiquitous genes. Furthermore, most of the insertions are determined to be in promoter regions, first exons, or first introns of genes. Thus many of the insertions are predicted to disrupt the function of these genes (see Example 29; Table 8). These genes are either known genes or predicted genes based on expressed sequence tag (EST) sequences. Preferred cell lines are those, which appear not to disrupt a gene, such as DOC1 or DOC33.

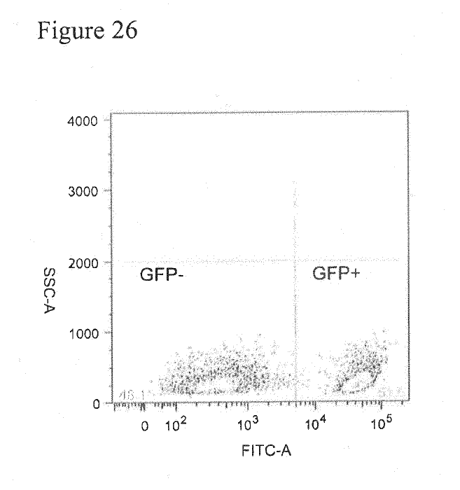

The CAG-EGFP-CAG-neo portion of the docking site can be deleted by Cre-lox recombination. After Cre-lox recombination, all that remains in the docking site is the HS4 insulators, the attP site, and the promoterless puro gene. This reduces the number of foreign proteins that are produced in the cells and transgenic chickens, which may have an effect on their health, particularly when expressed ubiquitously from a strong promoter such as CAG or B-actin. The Cre-lox recombination can be performed in cell culture, by transient transfection of the docking site clone with a circular Cre-expression vector. After several days, the culture is monitored for loss of EGFP expression caused by excision of the CAG-EGFP gene. About 50% of the cells no longer express EGFP, and these cells can be sorted by flow cytometry to purify them (FIG. 26).

Alternatively, Cre recombination can be performed by crossing the transgenic chickens carrying the docking site construct to chickens carrying the ERNI-Cre transgene (Cre4 birds).

When transgenic chickens bearing these docking site integrations are made, the resulting homozygous chickens may be healthy and fertile despite having an insertion in a gene. An example of such a line is the TP85 line (also called BN; see Example 26), with an insertion in the gene encoding aldehyde dehydrogenase 3 family member A2 on chicken chromsome 19. The construct was an HS4-insulated B-actin neo transgene, and it inserted into the promoter region within about 10 bp of the transcription start site of the gene. Birds homozygous for the insertion are healthy and fertile.

In some cases, however, the insertions may cause deleterious effects, such as developmental defects, anatomical or physiological defects, sterility, etc. See Example 27. Therefore it is important to validate a randomly inserted docking site insertion to make sure it does not cause any deleterious effects in the animal.

### EXAMPLE 31

### Establishment of a Docking Cell Line Consisting of More than IO.Skb of Expressed DNA

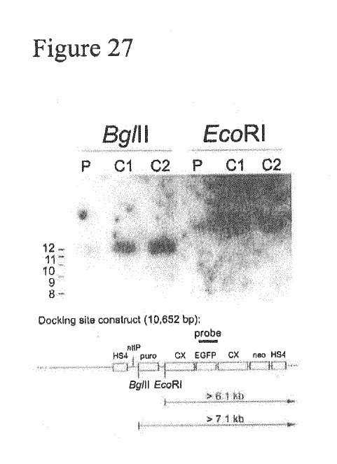

The Doc-1 cell line carries a transgene consisting of HS4; attP; promoterless puromycin resistance gene; loxP; CAG promoter; EGFP; CAG promoter; neomycin resistance gene; HS4 (see Example 29). The construct was linearized and transfected into cultured PGCs, and drug resistant colonies were obtained. These colonies were expanded for further analysis. The Doc-1 cell line was injected into recipient embryos and GO chimeric chicks were hatched. The roosters were grown to sexual maturity and their sperm was analyzed by FACS analysis for the presence of GFP positive sperm. Two roosters were selected for breeding and germline transmission rates were 3 and 8%. Blood was taken from GFP positive chicks and analyzed by Southern analysis that confirmed the presence of the docking site (FIG. 27)

### EXAMPLE 32

### Targeted Insertion of a Docking Site

To avoid the placement of authentic attP sites into CpG islands of genes, it is possible to use gene targeting to place an attP site into a pre-determined site in the chicken genome. A region of the genome is selected, homology arms are prepared by high-fidelity PCR or by genomic cloning in plasmid vectors, and the targeting vector is assembled with a selectable marker for transfection and selection of PGC clones.

### EXAMPLE 33

### Site-Specific Insertion Into a Cell Line Carrying a Docking Site

For insertion into a docking site, a circular construct containing an attB site is constructed. The attB-containing construct is similar to that used in the Example above, with the important difference that there is no selectable marker. Instead, a promoter (such as the ERNI promoter) is placed adjacent to the attB site, such that upon integration into the docking site, the promoter is placed in a position to drive expression of the selectable marker in the docking site.

The promoter-attB backbone can be used to select for insertion into the attP-promoterless puro docking site. The attB construct carries other genes of interest, such as tissue-specific promoters driving expression of genes encoding pharmaceutical proteins such as antibodies.

The functionality of the docking site and efficiency of integration in a docking site were tested in a PGC cell line containing the docking site. 5×10⁶ cells were co-transfected with 0.5 ug of a construct containing Erni-attB and 0.5 ug of a circular construct expressing integrase. After electroporation, the cells were replated into 48-lcm² wells to obtain single colonies. In 42 of 48 wells, colonies were observed.

PCR revealed that the ERNI-attB construct was correctly integrated into the docking site by amplification of the attL site produced by recombination of attB with attP. One primer was in the ERNI sequence and one primer was in the puromycin sequence, and amplification can only occur if the ERNI promoter has integrated upstream of the puromycin gene. Three primer sets were used and all produced positive results:
ERNI-37F+Mpuro-8R product size 152 bp
ERNI-133F+Mpuro-8R product size 248 bp
ERNI-342F+Mpuro-83R product size 522 bp

### Primer Sequences:

ERNI-37F
SEQ ID No: 44
ACCACGGCAACGGGAGAGGCTTAT
ERNI-133F
SEQ ID No: 32
TTGCTCAAGCCCCCAGGAATGTCA
ERNI-342F
SEQ ID No: 45
TGGGCAAAGGCAGAGGAATC
puro-8R
SEQ ID No: 33
CGAGGCGCACCGTGGGCTTGTA
puro-83R
SEQ ID No: 46
GCGTGGCGGGGTAGTCG

Four independent clones from the ERNI-attB transfection into DOC2 cells were tested by PCR and all four showed the correct size amplification products with all three primer sets. The PCR product generated with the ERNI-133F SEQ ID No: 32+puro-8R SEQ ID No: 33 primers was cloned and sequenced to verify that the PCR product was correct. The sequence matched perfectly to the expected attL sequence (the combination of attB and attP) and contained the expected partial ERNI and puromycin sequences. The integrase-mediated recombination crossover event thus occurred at the correct core nucleotides in the attB and attP sites and was verified as authentic.

### EXAMPLE 34

### Cre Effectively Recombines LoxP Sites in Chicken Embryos

The 10 Cre lines with intact pLenti-ERNI-Cre transgenes (and the one line with a rearranged ERNI-Cre transgene) were tested for Cre recombinase activity. Although the ERNI promoter was expected to drive high-level expression of the Cre recombinase in early embryos, transgenes can be silenced if they happen to integrate into an unfavorable region of the genome (a phenomenon known as ‘position effect’). Therefore it was important to determine the activity of Cre recombinase in all of our Cre lines in order to select a line or lines with the desired level of activity.

To determine the level of activity of our Cre transgenes, the ability of Cre to catalyze recombination of a loxP-Reaper transgene in doubly transgenic embryos carrying one copy of the Cre transgene and one copy of the loxP transgene was analyzed by Southern blot. The loxP-Reaper transgene contains a 1.4 kb sequence, called a STOP cassette, flanked by loxP sites in the same orientation. Recombination between the two loxP sites results in excision of the 1.4 kb intervening sequence from the chromosome, leaving behind a single loxP site. The intervening sequence is then lost since it is no longer linked to a chromosome. After excision, the loxP-Reaper transgene is reduced in size by 1.4 kb. A Southern blot assay was developed in which the reduction in size of the loxP-Reaper transgene is used to measure the Cre recombinase activity. Digestion with the restriction enzyme SacI produces a full-length (unrecombined) loxP-Reaper fragment of approximately 2.8 kb when hybridized to a probe consisting of the Reaper gene and portions of the Lentiviral vector backbone (the blasticidin gene and SV40 sequences). Upon Cre-mediated recombination and excision of the 1.4 kb STOP sequence, the Reaper SacI fragment is reduced in size to approximately 1.4 kb when hybridized to the same probe. The probe hybridizes to sequences that are not affected by Cre recombination, and thus it hybridizes equally to both full-length and recombined loxP-Reaper transgenes.

To estimate the level of Cre activity expressed by the various pLenti-ERNI-Cre transgenic lines, the ratio of the band intensities of the full-length (non-recombined) and the recombined transgenes is determined. If Cre is not active, then little or no recombined Reaper is observed, and only the full-length is observed. If Cre is moderately active, then both SacI fragments are observed, indicating that recombination occurred in some cells but not other cells. If Cre is very active, then only the recombined Reaper band is observed because the loxP-Reaper transgene has been recombined in every cell.

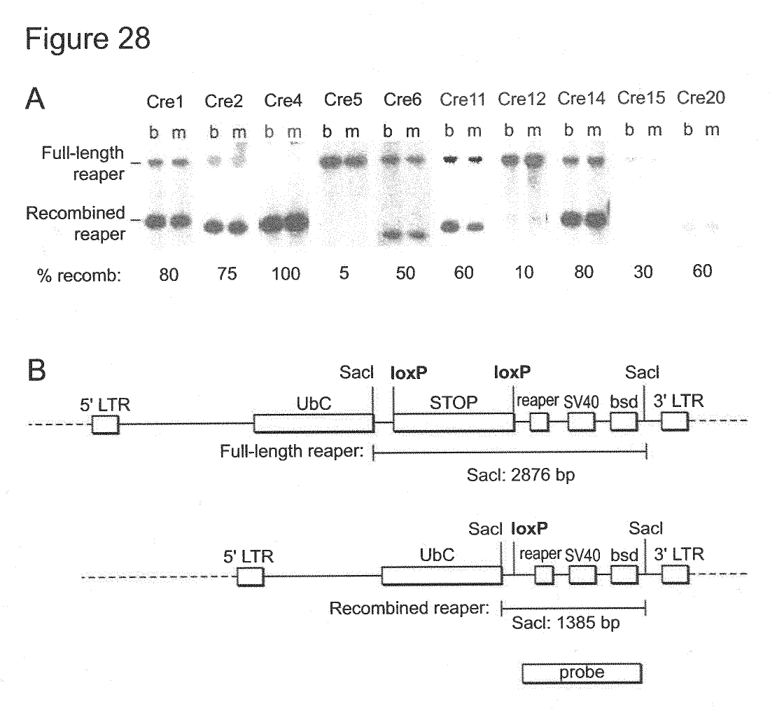

The data summarizing the activity of the Cre lines are presented in FIGS. 28A and 28B. Cre activity in the 11 lines tested was quite variable between lines, and in some cases within lines as well. Only one line out of 11 catalyzed 100% recombination of the loxP-Reaper transgene (the Cre4 line). In this line, every embryo tested (18 out of 18) displayed 100% recombination. In other lines, recombination levels ranged from about 5% up to about 80%. For several of these lines (Crel, Cre2, Crel 1 and Cre20) there was significant variability from embryo to embryo in the level of recombination. For example, Crel 1 and Cre20 catalyzed only about 10% recombination in some embryos but up to 60% in others. For most embryos, brain and skeletal muscle tissue were analyzed, and the level of recombination was similar in both tissues in every case. Since the ERNI promoter is thought to be active in neural tissue, it was expected that perhaps brain would show increased levels of recombination, but recombination in brain and muscle was always about the same. It is unclear whether the variation in Cre recombinase activity that is observed is caused by variation in the levels of Cre expression or by variation in the length of time that Cre activity is expressed. For example, in the Cre4 line it could be that Cre is continuously expressed at a low level, leading to accumulated recombination by day 15 of development of 100%, whereas the other lines may silence Cre expression at an earlier time before recombination occurs in every cell. Alternatively, Cre4 may express very high levels of Cre protein but only in early development, catalyzing 100% recombination.

The only line, which showed no recombination was the line with the rearranged or deleted transgene.

### EXAMPLE 35

### Successful Recombination of Three Chicken Lines Carrying Different Reaper Insertions

To show that the Cre recombinase in pLenti-ERNI-Cre transgenic chickens is capable of catalyzing recombination of different loxP substrates, the Cre4 line was crossed to three different loxP-Reaper lines (called 6-03, 6-51 and 9-51). The Cre4 line was chosen because it previously showed 100% recombination. Embryos were selected that inherited one of the loxP-Reaper transgenes and a copy of the Cre4 transgene.

To determine the level of recombination of the three loxP-Reaper transgenes, the ability of Cre to catalyze recombination in doubly transgenic embryos carrying one copy of the Cre4 transgene and one copy of the loxP-Reaper transgene was analyzed by Southern blot. The loxP-Reaper transgene contains a 1.4 kb sequence, called a STOP cassette, flanked by loxP sites in the same orientation. Recombination between the two loxP sites results in excision of the 1.4 kb intervening sequence from the chromosome, leaving behind a single loxP site. The intervening sequence is then lost since it is no longer linked to a chromosome. After excision, the loxP-Reaper transgene is reduced in size by 1.4 kb. A Southern blot assay was developed in which the reduction in size of the loxP-Reaper transgene is used to measure the Cre recombinase activity. Digestion with the restriction enzyme SacI produces a full-length (unrecombined) loxP-Reaper fragment of approximately 2.8 kb when hybridized to a probe consisting of the Reaper gene and portions of the Lentiviral vector backbone (the blasticidin gene and SV40 sequences). Upon Cre-mediated recombination and excision of the 1.4 kb STOP sequence, the Reaper SacI fragment is reduced in size to approximately 1.4 kb when hybridized to the same probe. The probe hybridizes to sequences that are not affected by Cre recombination, and thus it hybridizes equally to both full-length and recombined loxP-Reaper transgenes.

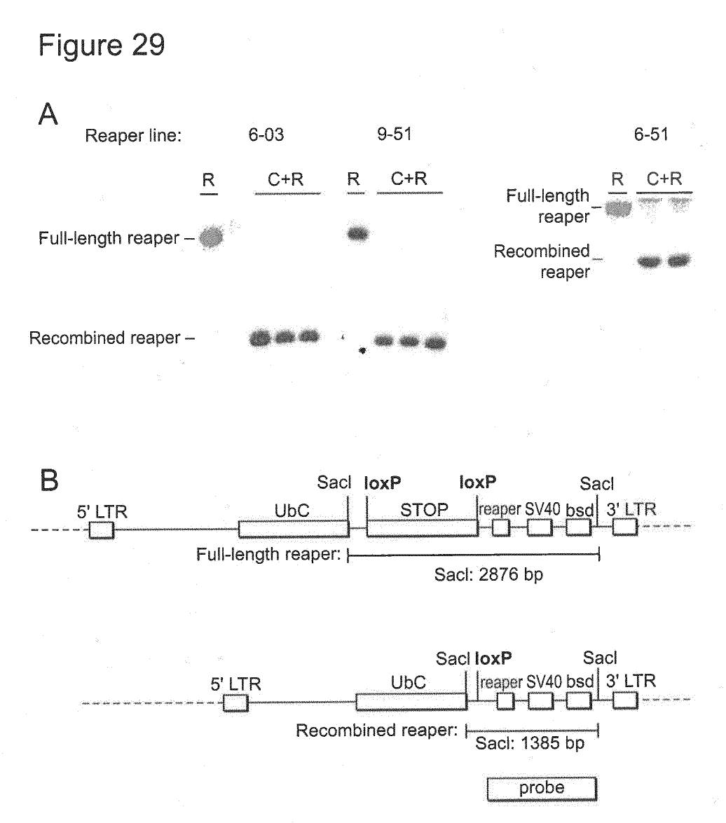

To estimate the amount of Cre-lox recombination in each of the loxP-Reaper lines, the ratio of the band intensities of the full-length (non-recombined) to recombined transgenes is determined. If Cre4 is capable of excising the STOP cassette in all three Reaper lines, then only the recombined Reaper band is observed because the loxP-Reaper transgene has been recombined in every cell. The results shown in FIG. 29A indicate that all three Reaper lines undergo 100% excision of the STOP cassette in the presence of the Cre4 transgene.

### EXAMPLE 37

### Cre-Mediated Excision of EGFP and Neo from Docking Site Integrated in the Chicken Genome

Cre recombination can be performed in vitro in cultured PGCs, as well as in transgenic birds. To perform Cre-lox recombination in cultured PGCs, the cells are transiently transfected with a Cre expression vector.

DOC2 cells were used for transfection with a Cre expression vector. This PGC line carries the docking site construct integrated in chromosome 21 in a CpG island linked to Prkz and several ESTs. All of the cells in the starting DOC2 culture were green fluorescent, since they carry the CX-EGFP gene in the docking site construct. Two Cre expression vectors were used: pBS185, with the Cre gene under the transcriptional control of the human CMV promoter, or an ERNI-Cre construct in which the ERNI promoter drives Cre expression.

The Cre expression constructs were transiently transfected into the DOC2 cells. After several days, the cultures were monitored for loss of green fluorescence, which was taken as an indicator that cells had taken up the Cre construct, expressed Cre, and Cre had caused excision of the sequences between the loxP sites on the docking site vector, including CX-EGFP-CX-neo. After Cre transfection, the culture consisted of green and non-green cells. To purify the two populations, the culture was sorted on the basis of green fluorescence by flow cytometry. Several million cells of each population (green and non-green) were collected.

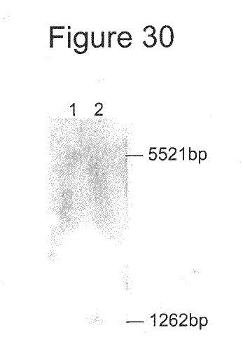

To prove that the EGFP gene had been excised in the non-green population of cells, Southern blot analysis was used (see FIG. 30). Genomic DNA from the two populations of cells (green and non-green) was prepared and digested with Hindlll restriction enzyme. The DNA was fractionated on an agarose gel, transferred to nylon membrane and hybridized with radiolabeled sequences from the puromycin resistance gene that is present in the docking site. The puro gene is in a region of the docking site construct that is not excised by Cre-lox recombination, and thus the puro probe will detect fragments in genomic DNA from both DOC2-excised and non-excised cells. The predicted size Hindlll fragments were: EGFP+ (non-excised), 5521 bp; EGFP-(excised), 1262 bp. The expected fragments were observed, indicating that in the non-green cells, Cre-lox recombination had resulted in deletion of the CX-EGFP-CX-neo sequences lying between the two loxP sites in the docking site construct integrated in DOC2 cells.

### EXAMPLE 38

### Preparation of IgL pKO5B Targeting Vector

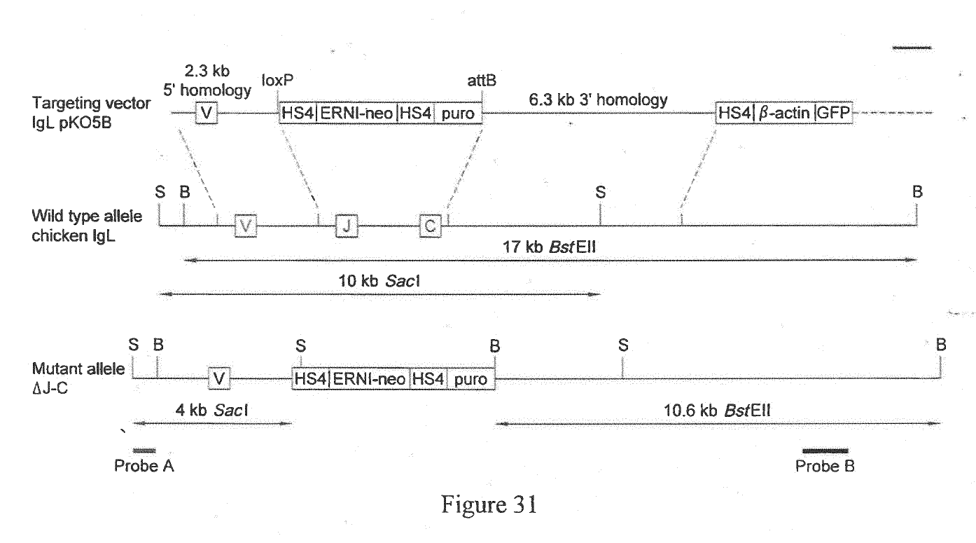

To target the chicken IgL locus, a targeting vector was prepared that deleted the endogenous J and C regions of the locus upon targeted integration (FIG. 31). The vector was the same as IgL pK05 previously described, except the selectable markers have been changed. The 5′ homology region on the vector consisted of a 2327 bp fragment in the vicinity of the IgL V region, and the 3′ homology region consisted of a 6346 bp fragment from downstream of the C region. The homology arms were cloned from isogenic DNA obtained from the cell line used in targeting transfections. The targeting construct contained one or more ways to disrupt expression, such as stop codon, nonsense sequences, attP site or combinations thereof. The vector also contained selectable marker genes and site-specific recombination sites. HS4 ERNI-neo: the 804 bp neomycin resistance gene was placed under the transcriptional control of the 800 bp ERNI promoter for expression in PGCs. ERNI expression in the chicken is limited to very early embryos and thus the selectable marker should not be expressed in adult chickens. The 250 bp core HS4 insulator element from the chicken β-globin locus was tandemly duplicated and the duplicated insulator was placed on both sides of the ERNI-neo selectable marker. A single loxP site (for Cre-mediated recombination) was cloned upstream of the HS4-ERNI-neo. loxP=ATAACTTCGTATAGCATACATTATACGAAGTTAT (SEQ ID. No. 47) attP-puro: the 600 bp puro gene was linked to a 43 bp attP site (for phiC31-mediated recombination), with no promoter. attP-puro was then cloned downstream of HS4-ERNI-neo. Together, the loxP-HS4-ERNI-neo-attP-puro selectable marker cassette is 4089 bp. attP=ACGCCCCCAACTGAGAGAACTCAAAGGTTACCCCAGTTGGGGC (SEQ ID. No. 48) 5′ homology arm: a 2327 bp fragment was generated by ligating a 1994 bp Ncol-BamHI fragment from the PGC35 IgL SacI clone+Ma 333 bp Notl-Ncol PCR product amplified from PGC35 genomic DNA (with the primers 5′-Notl TTCTTGCGGCCGCAGGGAGCCATAGCCTGCTCCCATCATGCCC (SEQ ID. No. 49) and 3′-NcoI, AGAGGAGCCCAGGCCATGGCGGAAT) (SEQ ID. No. 50)
The PCR fragment contained the overlapping genomic Ncol site to join the two fragments together.
The resulting 2327 bp fragment was released with Notl and BamHI for cloning into the pKO vector backbone, upstream of the HS4 ERNI-puro. The Notl site was not present in the genome but added by PCR.
3′ homology arm: a 6346 kb Spel-Bglll fragment was generated by ligation of the following three fragments together: a Spel-EcoRI fragment from the SacI genomic clone, plus an EcoRI-ApaLI clone from EcoRI-Mfel genomic clone, plus a 300 bp ApaLI-Bglll PCR fragment amplified from the EcoRI-Mfel clone (the the primers 5′ ApaL AGTGCAGCTGCAGTGCACGGTA (SEQ ID. No. 51) and 3′-Bglll TTCTTAGATCTGTGACAAGCAGTCTCCGGTTAACA (SEQ ID. No. 52) The Bglll site was not present in the genome but added by PCR. The 3′ homology arm was cloned into the pKO vector backbone in between the HS4 ERNI-puro and the HS4 b-actin EGFP. HS4 b-actin EGFP: the 1.3 kb chicken b-actin promoter was used to drive expression of the 700 bp EGFP gene. One copy of the duplicated HS4 insulator was added at the end, to insulate the randomly inserted targeting vector from position effect and permit EGFP expression.
The final IgL pK05B targeting vector has a size of 17,681 bp and was linearized with Notl before transfection into PGCs.

### EXAMPLE 39

### Transfection of PGCs and Generation of KO-07 IgL Knockout PGC Cell Line

Thirty-eight aliquots of 5×10⁶ cells in 100 ul of electroporation buffer (V buffer from Amaxa) were transfected with 10 μg DNA each. All aliquots were electroporated with the Amaxa nucleofector pulse A33 (Amaxa). Nine clones were obtained, of which 4 were GFP-positive and not further pursued. The five non-GFP expressing clones were expanded for Southern analysis and given the names KO-07, 08, 09, 10 and 11.

### EXAMPLE 40

### Southern Blot Analysis

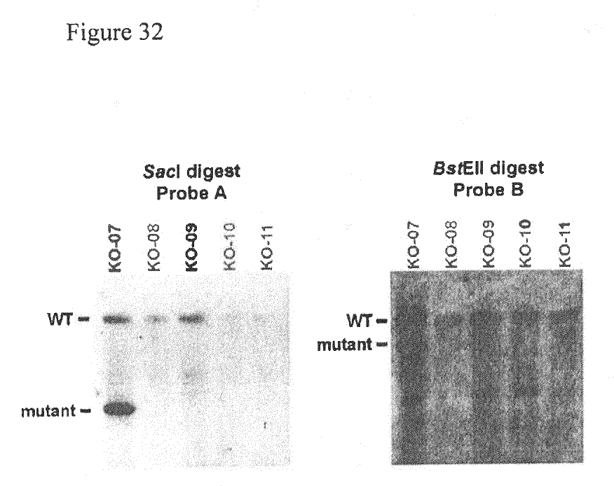

For the 5′ side of homologous recombination, genomic DNA from the five clonal PGC lines transfected with IgL pK05B was digested with SacI restriction enzyme and fractionated on 0.7% agarose gels. DNA was transferred to Nylon membrane and hybridized with a probe from the chicken IgL locus upstream from the regions used as the homology arms (i.e. an external probe). The probe is a 0.5 kb Sacl-BstEII fragment and detects a wild type fragment of approximately 10 kb and a mutant fragment of approximately 4 kb. (FIG. 32, left panel) For the 3′ side of targeting, genomic DNA was digested with BstEU and the blot was hybridized with a 3′ 1.7 kb Nsil-Mfel fragment, also external to the targeting vector. (FIG. 32, right panel)

### EXAMPLE 41

### Production of Germline Chimeras

3000 PGCs were injected per embryo at Stage 15-16 (Hamburger&Hamilton) into the dorsal aorta. Embryos were incubated in surrogate shells. Hatched chicks were grown up to sexual maturity.

Chimeric roosters were mated to wild type Barred Rock hens by artificial insemination. Semen was collected from 9 roosters and used to inseminate hens. Six of the roosters transmitted the black feather phenotype to offspring, indicating germline transmission of the IgL knockout PGCs (Table 1). One of the roosters (IV75-41) transmitted at a rate over 50%.

TABLE 15
Germline transmission rates of the IgL knockout in 9 chimeric roosters
made with IgL knockout PGCs. The number of black progeny sired
by each rooster (indicating germline transmission of the knockout
PGCs) and the number of white progeny (from host PGCs) are listed.
Those roosters that sired black progeny are shown in bold font.

| | | | Percentage |
|---|---|---|---|
| | Number | Number | Germline |
| Rooster | black | white | transmission |
| IV75-26 | 3 | 30 | 9% |
| IV75-28 | 0 | 40 | 0 |
| IV75-41 | 23 | 19 | 55% |
| IV75-42 | 1 | 47 | 2.1% |
| IV75-48 | 1 | 53 | 1.8% |
| IV75-49 | 1 | 72 | 1.4% |
| IV75-58 | 0 | 9 | 0 |
| IV75-59 | 0 | 60 | 0 |
| IV75-81 | 3 | 13 | 19 |

### EXAMPLE 42

### Southern Blot Analysis of Knockout Embryos

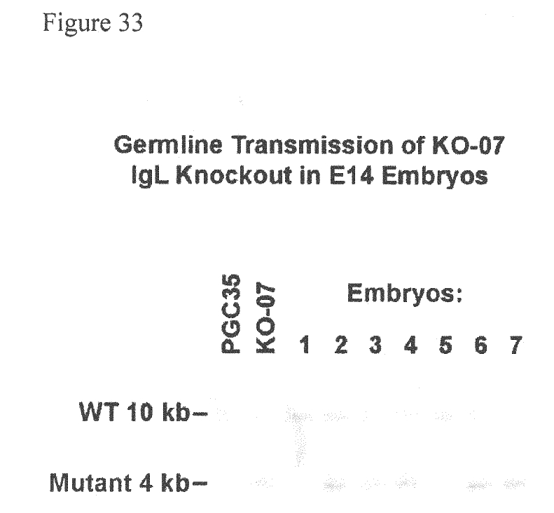

Black-feathered embryos at day 14 of development were euthanized and genomic DNA was prepared from skeletal muscle. Southern blots were performed showing that the knockout was transmitted to 5 of the 7 embryos tested in the experiment (embryos 2,3, 4, 6, and 7). Embryos 1 and 5 were wild type embryos that inherited the wild type IgL allele from the heterozygous, targeted KO-07 knockout PGCs (FIG. 33).

1. A transgenic chicken whose genome comprises a targeting construct that functionally disrupts the J and C genes of an endogenous immunoglobulin light chain gene,
wherein the transgenic chicken is derived from a clonal transfected primordial germ cell (PGC) whose genome comprises a targeting construct functionally disrupting the J and C gene segments of an endogenous immunoglobulin light chain gene, and an exogenous promoter operatively linked to a transgene,
wherein the clonal transfected PGC is derived from a clonal culture comprising at least 1×10⁵ of the transfected PGCs locus.

2.-6. (canceled)

7. The transgenic chicken of claim 1 wherein the targeting construct inserts two loxP sites in the same orientation, a stop codon, an att-p site, a non-sense sequence or combinations thereof into the endogenous immunoglobulin light chain gene.

8. The transgenic chicken of claim 1, wherein the targeting construct is comprised of two regions of homology to the endogenous immunoglobulin light chain gene and a selectable marker located between the regions of homology.

9. (canceled)

10. The transgenic chicken of claim 1 wherein the endogenous immunoglobulin light chain gene comprises a deletion of at least 10 kb.

11. (canceled)

12. A method to produce a transgenic chicken, comprising:
incorporating a targeting construct into a cultured chicken primordial germ cell (PGC) such that the targeting construct integrates into the genome of the PGC and disrupts at least a portion of the J and C region an endogenous immunoglobulin light chain gene by stable integration into the genome of the PGC,
wherein the targeting construct comprises a nucleotide sequence encoding the J and C segment of the immunoglobulin light chain, a nucleotide sequence encoding an exogenous promoter used to drive expression of a transgene, and a nucleotide sequence encoding the transgene, thereby producing a transfected PGC;
selecting a transfected PGC whose genome stably comprises the targeting construct stably, wherein said selection comprises maintaining the transfected PGCs in vitro under selection for at least 19 days and expanding the transfected PGC in culture to thereby produce a culture comprising at least 1×10⁵ clonal transfected PGCs;
inserting the clonal transfected PGCs into a recipient chicken embryo, and
hatching a chimeric chicken from the embryo, wherein the chicken comprises germline cell derived from the clonal transfected PGCs which comprises the targeting construct disrupting the J and C region of the endogenous immunoglobulin light chain gene and the promoter operatively linked to the transgene, and
breeding the chimeric chicken to produce a transgenic chicken, wherein the genome of the transgenic chicken is derived from the germline cell and comprises the targeting construct disrupting the J and C region of the endogenous immunoglobulin light chain gene and the promoter operatively linked to the transgene.

13.-16. (canceled)

17. The method of claim 11 wherein the targeting construct inserts a stop codon, two loxP sites in the same orientation, non-sense sequences, an att-p site or combination thereof into the light chain immunoglobulin gene.

18.-21. (canceled)

22. The method of claim 1, wherein the targeting construct is comprised of at least two regions of homology to the endogenous immunoglobulin light chain gene.

23. The method of claim 22, wherein the integration of the targeting construct into the genome of the cell places a selectable marker located between two regions of homology between the targeting construct and the endogenous gene.

24. The method of claim 1, wherein the targeting construct is comprised of a positive selection marker.

25. The method of claim 1, wherein the targeting construct is comprised of a negative selection marker.
